(19) United States
(12) Patent Application Publication
(10) Pub. No.: US 2023/0090691 A1
(43) Pub. Date: Mar. 23, 2023

(54) COMPOSITIONS AND METHODS FOR PROMOTING AND/OR MAINTAINING VAGINOVULVAL AND PERIANAL TISSUE VITALITY AND TISSUE HEALTH

(71) Applicant: SPINART, LLC, Hazlet, NJ (US)

(72) Inventor: Garrett Reynolds, Hazlet, NJ (US)

(73) Assignee: SPINART, LLC, Hazlet, NJ (US)

(21) Appl. No.: 17/900,606

(22) Filed: Aug. 31, 2022

Related U.S. Application Data
(60) Provisional application No. 63/239,755, filed on Sep. 1, 2021.

(51) Int. Cl. A61K 8/73; A61K 8/04; A61K 8/34; A61K 8/44; A61K 8/368; A61K 8/365; A61Q 19/00

(52) U.S. Cl. CPC A61K 8/735; A61K 8/042; A61K 8/347; A61K 8/44; A61K 8/368; A61K 8/365; A61Q 19/00

(57) ABSTRACT

The described invention provides a cosmetic composition formulated for topical application containing a water-based gel component comprising hyaluronic acid; a botanical ingredient component comprising decarboxylated cannabidiol (CBD) representing about 20% of an isolate, and a cosmetic composition stabilizing system comprising arginine, p-anisic acid, and levulinic acid, wherein the composition is a slightly viscous non-occluded water based liquid; pH of the finished product ranges from about 4.0 to about 5.0; and the composition is not psychoactive. The described invention also provides methods for promoting and maintaining vaginovulval and perianal tissue vitality and tissue health in a subject in need thereof by administering the composition topically to the subject.

### CROSS-REFERENCE TO RELATED APPLICATIONS

This application claims the benefit of priority to provisional application 63/239,755, filed on Sep. 1, 2021, the contents of which is incorporated by reference in its entirety.

### FIELD OF THE INVENTION

The described invention relates to topical compositions, wound healing, tissue repair and rejuvenation.

### BACKGROUND OF THE INVENTION

### Mechanisms of Wound Healing

The term “wound healing” refers to the process by which the body repairs trauma to any of its tissues, especially those caused by physical means and with interruption of continuity.

A wound-healing response often is described as having three distinct phases: injury, inflammation and repair. Generally speaking, the body responds to injury with an inflammatory response, which is crucial to maintaining the health and integrity of an organism. If however it goes awry, it can result in tissue destruction.

### Phase I: Injury

Injury caused by factors including, but not limited to, autoimmune or allergic reactions, environmental particulates, infection or mechanical damage often results in the disruption of normal tissue architecture, initiating a healing response. Damaged epithelial and endothelial cells must be replaced to maintain barrier function and integrity and prevent blood loss, respectively. Acute damage to endothelial cells leads to the release of inflammatory mediators and initiation of an anti-fibrinolytic coagulation cascade, temporarily plugging the damaged vessel with a platelet and fibrin-rich clot.

Platelet recruitment, degranulation and clot formation rapidly progress into a phase of vasoconstriction with increased permeability, allowing the extravasation (movement of white blood cells from the capillaries to the tissues surrounding them) and direct recruitment of leukocytes to the injured site. The basement membrane, which forms the extracellular matrix underlying the epithelium and endothelium of parenchymal tissue, precludes direct access to the damaged tissue. To disrupt this physical barrier, zinc-dependent endopeptidases, also called matrix metalloproteinases (MMPs), cleave one or more extracellular matrix constituents allowing extravasation of cells into, and out of, damaged sites. Specifically, MMP-2 (gelatinase A, Type N collagenase) and MMP-9 (gelatinase B, Type IV collagenase) cleave type N collagens and gelatin, two important constituents of the basement membrane. Recent studies have found that MMP-2 and MMP-9 are upregulated, highlighting that tissue-destructive and regenerative processes are common in fibrotic conditions. The activities of MMPs are controlled by several mechanisms including transcriptional regulation, proenzyme regulation, and specific tissue inhibitors of MMPs. The balance between MMPs and the various inhibitory mechanisms can regulate inflammation and determine the net amount of collagen deposited during the healing response.

### Phase II: Inflammation

Once access to the site of tissue damage has been achieved, chemokine gradients recruit inflammatory cells. Neutrophils, eosinophils, lymphocytes, and macrophages are observed at sites of acute injury with cell debris and areas of necrosis cleared by phagocytes.

The early recruitment of eosinophils, neutrophils, lymphocytes, and macrophages providing inflammatory cytokines and chemokines can contribute to local TGF-β and IL-13 accumulation. Following the initial insult and wave of inflammatory cells, a late-stage recruitment of inflammatory cells may assist in phagocytosis, in clearing cell debris, and in controlling excessive cellular proliferation, which together may contribute to normal healing. Late-stage inflammation may serve an anti-fibrotic role and may be required for successful resolution of wound-healing responses. For example, a late-phase inflammatory profile rich in phagocytic macrophages, assisting in fibroblast clearance, in addition to IL-10-secreting regulatory T cells, suppressing local chemokine production and TGF-β, may prevent excessive fibroblast activation.

The nature of the insult or causative agent often dictates the character of the ensuing inflammatory response. For example, exogenous stimuli like pathogen-associated molecular patterns (PAMPs) are recognized by pathogen recognition receptors, such as toll-like receptors and NOD-like receptors (cytoplasmic proteins that have a variety of functions in regulation of inflammatory and apoptotic responses), and influence the response of cells involved in the innate immune response to invading pathogens. Endogenous danger signals also can influence local innate cells and orchestrate the inflammatory cascade.

The nature of the inflammatory response dramatically influences resident tissue cells and the ensuing inflammatory cells. Inflammatory cells themselves also propagate further inflammation through the secretion of chemokines, cytokines, and growth factors. Many cytokines are involved throughout a wound-healing and fibrotic response, with specific groups of genes activated in various conditions. For example, chronic allergic airway disease in asthmatics is associated commonly with elevated type-2 helper T cell (Th₂) related cytokine profiles (including, but not limited to, interleukin-4 (IL-4), interleukin-5 (IL-5), interleukin-6 (IL-6), interleukin-13 (IL-13), and interleukin-9 (IL-9)), whereas chronic obstructive pulmonary disease and fibrotic lung disease (such as idiopathic pulmonary fibrosis) patients more frequently present pro-inflammatory cytokine profiles (including, but not limited to, interleukin-1 alpha (IL-1α), interleukin-1 beta (IL-1β), interleukin-6 (IL-6), tumor necrosis factor alpha (TNF-α), transforming growth factor beta (TGF-β), and platelet-derived growth factors (PDGFs)). Each of these cytokines has been shown to exhibit significant pro-fibrotic activity, acting through the recruitment, activation and proliferation of fibroblasts, macrophages, and myofibroblasts.

### Phase III: Tissue Repair and Contraction

The closing phase of wound healing consists of an orchestrated cellular re-organization guided by a fibrin (a fibrous protein that is polymerized to form a “mesh” that forms a clot over a wound site)-rich scaffold formation, wound contraction, closure and re-epithelialization. The vast majority of studies elucidating the processes involved in this phase of wound repair have come from dermal wound studies and in vitro systems.

Myofibroblast-derived collagens and smooth muscle actin (α-SMA) form the provisional extracellular matrix, with macrophage, platelet, and fibroblast-derived fibronectin forming a fibrin scaffold. Collectively, these structures are commonly referred to as granulation tissues.

In addition to fibronectin, the provisional extracellular matrix consists of glycoproteins (such as PDGF), glycosaminoglycans (such as hyaluronic acid), proteoglycans and elastin. Growth factor and TGF-β-activated fibroblasts migrate along the extracellular matrix network and repair the wound. Within skin wounds, TGF-β also induces a contractile response, regulating the orientation of collagen fibers. Fibroblast to myofibroblast differentiation, as discussed above, also creates stress fibers and the neo-expression of α-SMA, both of which confer the high contractile activity within myofibroblasts. The attachment of myofibroblasts to the extracellular matrix at specialized sites called the “fibronexus” or “super mature focal adhesions” pull the wound together, reducing the size of the lesion during the contraction phase. The extent of extracellular matrix laid down and the quantity of activated myofibroblasts determines the amount of collagen deposition. To this end, the balance of matrix metalloproteinases (MMPs) to tissue inhibitor of metalloproteinases (TIMPs) and collagens to collagenases vary throughout the response, shifting from pro-synthesis and increased collagen deposition towards a controlled balance, with no net increase in collagen. For successful wound healing, this balance often occurs when fibroblasts undergo apoptosis, inflammation begins to subside, and granulation tissue recedes, leaving a collagen-rich lesion.

The removal of inflammatory cells, and especially α-SMA-positive myofibroblasts, is essential to terminate collagen deposition. From skin studies, re-epithelialization of the wound site re-establishes the barrier function and allows encapsulated cellular re-organization. Several in vitro and in vivo models, using human or rat epithelial cells grown over a collagen matrix, or tracheal wounds in vivo, have been used to identify significant stages of cell migration, proliferation, and cell spreading. Rapid and dynamic motility and proliferation, with epithelial restitution from the edges of the denuded area occur within hours of the initial wound. In addition, sliding sheets of epithelial cells can migrate over the injured area assisting wound coverage. Several factors have been shown to regulate re-epithelialization, including serum-derived transforming growth factor alpha (TGF-α), and matrix met alloproteinase-7 (MMP-7) (which itself is regulated by TIMP-1).

Collectively, the degree of inflammation, angiogenesis, and amount of extracellular matrix deposition all contribute to ultimate development of a fibrotic lesion or scar. Thus, therapeutic intervention that interferes with fibroblast activation, proliferation, or apoptosis requires an understanding and appreciation of all of the phases of wound repair. Although these three phases are often presented sequentially, during chronic or repeated injury these processes function in parallel, placing significant demands on regulatory mechanisms. (Wilson and Wynn, Mucosal Immunol., 2009, 3(2): 103-121).

### Cutaneous Symptoms of Hemorrhoidal Disease

One in three Americans has hemorrhoids on screening colonoscopy [Sandler, R. S. and Peery, A F, Clin. Gastroenterol. Hepatol. (2019) 17 (1): 8-15, citing Everhart, J E and Ruhl, C E. Gastroenterology (2009) 136: 741-54]. Despite being prevalent and increasingly treated, symptomatic hemorrhoids are poorly understood with little evidence to guide treatment.

Hemorrhoids are clusters of vascular tissue, smooth muscle, and connective tissue arranged in three columns along the anal canal [Id., citing Sun, Z. and Migaly, J. Clin. Colon. Rectal Surg. (2016) 29: 22-9] They are present in healthy individuals as cushions that help to maintain continence [Id., citing Ganz, R A. Clin. Gastroenterol. Hepatol. (2013) 11: 593-603]. Although hemorrhoids are normal structures [Id., citing Haas, P A, et al. Dis. Colon Rectum (1984) 27: 442-50] the term hemorrhoid has come to refer to a pathologic or symptomatic process [Id., citing Sun, Z & Migaly, J. Clin. Colon Rectal Surg. (2016) 29: 22-9]

Internal hemorrhoids, which are located above the dentate line, are covered by columnar epithelium innervated by visceral nerve fibers that are not associated with pain. Internal hemorrhoids are graded based on the extent of prolapse.

External hemorrhoids lie below the dentate line. They are covered with squamous epithelium and are innervated by somatic nerves that can produce pain. External hemorrhoids are generally asymptomatic unless they thrombose. Thrombosed hemorrhoids are acutely painful. [Id., citing Madoff, R D and Fleshman, J W. Gastroenterology (2004) 126: 1463-73]. When external hemorrhoids resolve, skin tags may persist that can become irritated or create problems with hygiene.

The severity of hemorrhoids is classified into four stages, according to Goligher's classification Hemorrhoids are graded based on degree of prolapse. Grade I do not prolapse below the dentate line and are visible on anoscopy or colonoscopy. Grade II prolapse below the dentate line but reduce spontaneously. Grade III prolapse and require manual reduction. Grade IV prolapse and remain below the dentate line. They are not reducible.

The etiology of hemorrhoids is uncertain. Hemorrhoids are commonly reported in women mostly during pregnancy and after vaginal birth. It is generally believed that pregnancy and vaginal birth predispose women to develop symptomatic hemorrhoids for several reasons: hormonal changes, increased intra-abdominal pressure, straining during defecation due to constipation, prolonged straining during the second stage of labor for more than 20 minutes, and giving birth to a baby with a weight over 3800 g [Kestranek, J. Drugs in Context (2019) 8: 212602]. However, one report found that overweight and pregnancy were not associated with current hemorrhoids [Id., citing Peery, A F et al. PLoS One (2015) 10: e0139100]. Others have found an association with BMI but not with age or pregnancy [Id., citing Riss, S. et al. Intl J. Colorectal Disease (2011) 27: 215-220].

Instructions to patients published in JAMA state: “anything that puts pressure on the veins in the lower body can lead to hemorrhoids, including straining during a bowel movement; sitting on the toilet for long periods; constipation or diarrhea; being overweight; pregnancy; and age, which causes tissues to become weaker.” [Sandler, R. S. and Peery, A F, Clin. Gastroenterol. Hepatol. (2019) 17 (1): 8-15, citing Sugarman, D T. JAMA (2014) 312: 2698].

Symptoms attributed to hemorrhoids include bleeding, pain, pruritus, fecal seepage, prolapse and mucus discharge [Id., citing Ganz, R A. Clin. Gastroentrol. Hepatol. (2013) 11: 593-603] However, a 2001 study from Germany of 458 patients referred with abdominal and/or anal symptoms supports the idea that symptoms linked to hemorrhoids may have other causes.

Treatments for hemorrhoids include medical therapies, non-surgical office based treatments and surgery [Id., citing Madoff, R D and Fleshman, J W. Gastroenterology (2004) 126: 1463-73].

Dietary and lifestyle changes, which require high patient compliance, are usually considered the first step for any conservative strategy; however, evidence supporting these interventions is anecdotal [Kestranek, J. Drugs in Context (2019) 8: 212602]. Since inflammation plays an important role especially in the cutaneous symptoms of hemorrhoidal disease [Id., citing Abramowitz, L. et al. Aliment. Pharmacol. Ther. (2010); 31 (Suppl. 1) 1-58], medical treatment is mainly based on the use of topical preparations containing anti-inflammatory drugs, including steroids, anesthetics, astringents and/or antiseptics [Id., citing Davis, B R et al. Dis. Colon Rectum. (2018) 61: 284-92]. However, in most cases no randomized trials have been conducted to assess the efficacy and safety of different interventions which, in some cases, e.g., steroids, are associated with the potential onset of adverse events. [Id., citing Brown, S R. Ther. Adv. Chronic Dis. (2017) 8 (10): 141-47; Davis, B R et al. Dis. Colon Rectum (2018) 61: 284-92].

Some commonly used combinations include ketocaine/fluocinolone and hydrocortisone/benzocaine. While corticosteroids can be effective in this scenario, these molecules, often available as prodrugs, may be associated with a risk of systemic adsorption being systemically absorbed, distributed, metabolized, and excreted. Thus, their higher lipophilicity may limit their application over the middle-term period or in women who are elderly, are breastfeeding, or are pregnant.

The topical combination of tribenoside and lidocaine (marketed under the brand Procto-Glyvenol®, Recordati SpA, Italy) is a medical preparation for the local treatment of hemorrhoids that has been used for decades in the therapy of hemorrhoids on patients of either gender [Lorenc, Z. Gokce, O. Eur. Review Medical & Pharmacological Sciences (2016) 20: 2742-51]. It is delivered as a suppository or rectal cream. Its efficacy and safety are supported by a number of well-conducted studies and broad clinical experience. This product combines the rapid local anesthetic action exerted by lidocaine (to provide rapid relief from pain and itching), with the efficacy of tribenoside (a saccharide derivative reported to interact with epidermal cells and to regulate expression and localization of laminins, thus helping reconstruct basement membrane in wound healing of hemorrhoids) in reducing inflammation, promoting local healing, and favoring the recovery of local vessels to normal conditions. This double mechanism of action purportedly allows control of both subjective (e.g., pain and discomfort) and objective (e.g., prolapse and bleeding) symptoms of hemorrhoids.

There are more than 100 hemorrhoid remedies listed on Amazon.com, many with inflated claims of benefit or dubious ingredients. [Sandler, R. S. and Peery, A F, Clin. Gastroenterol. Hepatol. (2019) 17 (1): 8-15]:

Topical formulations providing alternatives to corticosteroids, but still endowed with anti-inflammatory and wound-healing effects, therefore would be highly desirable.

### Sexual Side Effects of Cancer Treatment

Radiation to the pelvic area can affect a woman's sexual health during and after treatment, because the radiation beams damage the delicate tissue in and around female genitals. Certain types of cancers are treated with a radiation implant, which is a radiation source put inside the bladder, uterus or vagina for a certain number of days. During treatment, tissues in the treatment area can get irritated; they may become pink and swollen and may look sunburned. A woman's vagina may feel tender during radiation treatment and for a few weeks afterward. Radiation to the vagina can also damage its lining, making it thin and fragile. In rare cases, vaginal ulcers or open sores may develop, which may take several months to heal after radiation therapy ends. When treatment ends and the irritation heals, there might be scarring, and the walls of the vagina may become leathery and tough. Radiation treatment can also shorten or narrow the vagina, which means that the walls might not stretch out as much during sex, which can cause pain. In some cases, the bladder and bowel are damaged, which can also impact sexual health.

Many of these side effects become a chronic condition that tends to worsen throughout the years. They therefore requires prompt and long-term therapy to achieve good results and to avoid the recurrence of symptoms when treatment is stopped.

Women with the greatest risk of sexual side effects of cancer treatment include those being treated for bladder cancer, breast cancer, gynecologic cancers (e.g., cervical cancer, endometrial cancer, ovarian cancer, vaginal cancer, vulvar cancer), colon cancer, rectal cancer, and uterine cancer.

### Breast Cancer

Because of declining mortality rates, patients that survive breast cancer require ongoing management of sequelae from the disease or its treatment. Breast cancer survivors may have a variety of symptoms related to low estrogen levels as a result of chemotherapy-induced ovarian failure or antiestrogen hormonal therapy, including, without limitation, hot flashes, dyspareunia, vaginal dryness and urogenital atrophy.

Urogenital tract atrophy is a manifestation of estrogen deprivation in breast cancer patients who have received chemotherapy or endocrine therapy, which may induce various symptoms of the vulvovaginal area including vaginal dryness, burning, itching, dyspareunia and abnormal discharge. Lee, Y-K et al. Obstet. Gynecol. (2011) 117: 922-7]. While symptoms of urogenital atrophy are common in breast cancer survivors. its optimal management remains unknown. Although estrogen replacement could resolve urogenital symptoms, it is contraindicated in breast cancer patients, and even local administration of estradiol tablets could affect serum hormone levels. [Id., citing Beral, V. Lancet (2003) 362: 419-27; Rosenberg, L U et al. Breast Cancer Res. (2006) 8: R11].

Chemotherapy drugs commonly irritate all mucous membranes in the body, including vulvar and vaginal tissues. The irritation can cause burning and inflammation. Drugs that impact hormone production and absorption, such as Tamoxifen and various aromatase inhibits, also change the body's use of estrogen and cause vaginal health issues.

For vaginal dryness and dyspareunia, guidelines suggest the use of vaginal lubricants for sexual intercourse and vaginal moisturizers for general comfort. [Zoberi, K. & Tucker J. Am. Fam. Physician (2019) 99 (6): 370-75]. A systematic review of randomized controlled trials (RCTs) evaluating treatment options for urogenital atrophy in breast cancer patients was reported by Mazzarelio, S. et al. Breast Cancer Res. Treat. (2015) 152 (1): 1-8. An electronic literature search was performed to seek relevant citations from EMBASE, Ovid Medline and the Cochrane Library from 1946 to November 2014. There were no restrictions in terms of disease status (early vs. metastatic stage), patient age and prior anti-cancer treatment. Interventions included a pH-balanced gel, Replens® (a vaginal moisturizer containing polycarbophil, mineral oil, hydrogenated palm oil glyceride, glycerine, carbomer homopolymer type B, sodium hydroxide and sorbic acid), lidocaine, Estring® (an estradiol vaginal ring comprising silicone polymers and barium sulfate) and Vagifem® (a film-coated tablet containing 10 μg estradiol as estradiol hemihydrate, hypromellose, lactose monohydrate, maize starch and magnesium stearate). All doses, preparations and frequency of administration were considered. Outcomes included improvements in both vaginal symptoms (e.g., dryness, pain, dyspareunia and itching) and vaginal hormone response measured by validated scales [e.g., Vaginal Health Index (VHI) and Vaginal Maturation Index (VMI)]. Of 430 unique citations identified, 4 studies (n=196) met inclusion criteria. Sample sizes ranged from 7 to 98 patients. Given the heterogeneity of the studies, a narrative synthesis of results was performed using VHI score. One study of 98 patients suggested that vaginal pH-balanced gel (mean VHI 5.00±0.816, mean VMI 51.18±3.753) was more efficient than placebo (VHI 16.98±3.875, p<0.001, VMI 47.87±2.728, p<0.001) at 12 weeks in providing vaginal symptom relief. In patients who used lidocaine, 90% had reduced dyspareunia compared to saline in a study of 46 patients. Although increased serum estradiol occurred, both Estring® and Vagifem® were shown to improve quality of life and VMI in a study of seven patients.

### Gynecologic Cancers

The treatment of gynecologic malignancy is associated with many causes of genito-pelvic pain and dyspareunia. Many of these symptoms can arise from alterations in vaginal health from surgery, radiation and chemotherapy causing vaginal shortening, stenosis, atrophy and dryness.

### Endometrial Cancer

Endometrial cancer is the most common gynecologic malignancy in Western countries. The majority occur in postmenopausal women and surgery is the primary treatment for most patients. Even in the absence of adjuvant therapy in the form of radiotherapy and/or chemotherapy, patients are at risk of experiencing sexual dysfunction. [Huffman, L B e al. Gynecol. Oncol. (2016) 140(2): 359-68]A prospective evaluation of the prevalence of sexual dysfunction in early-stage (I-IIIa) endometrial cancer patients 1 to 5 years from primary surgical treatment (N=72 showed that 89% of participants had some form of sexual dysfunction determined by the Female Sexual Function Index (FSFI) score of <26, and pain was the most commonly affected domain. Only 18% of participants received adjuvant radiation therapy. For patients with higher risk of recurrence and higher stage disease, adjuvant therapy in the form of radiotherapy and/or chemotherapy typically is recommended. The Post-Operative Radiotherapy in Endometrial Cancer (PORTEC-2) study investigated the outcomes and adverse effects of vaginal brachytherapy (VBT) compared to external beam radiotherapy (EBRT) for the treatment of high-intermediate risk endometrial cancer [Id., citing Nout, R A et al. The Lancet (2010) 375: 816-23]. While there were no differences in sexual function between VBT and EBRT patients, when compared to an age-matched control population, participants in the study reported significantly more vaginal dryness and lower sexual interest, activity and enjoyment. [Id., citing Id., citing Nout, R A et al. Eur. J. Cancer (2012) 48 (11): 1638-48]. In another study, vaginal changes following radiation included vaginal stenosis (meaning narrowing), vaginal scarring, mucosal telangiectasia (meaning dilation of previously existing small or terminal vessels located near the surface of mucosal membranes; commonly known as spider veins) and mucosal atrophy (meaning thinning, drying and inflammation of the vaginal walls that may occur when the body has less estrogen) [Id., citing Nunns, D. J. Gynecological Cancer (2000) 10 (3): 233-38].

### Cervical Cancer

The surgical treatment of early-stage cervical cancer can include cervical conization, simple hysterectomy, or radical hysterectomy with pelvic lymphadenectomy. Radical hysterectomy is associated with negative effects on sexual health and quality of life [Id., citing Greimel E R, et al. Psychoncology. (2009) 18(5):476-82]. Persistent sexual health concerns include lack of sexual interest (25%), lymphedema (19%), genital numbness (71%), and insufficient lubrication (24%). [Id., citing Jensen P T, et al. Cancer. (2004) 100(1):97-10625-27; Pieterse, Q D et al. Int J Gynecol Cancer. (2013) 23(9):1717-25; Pieterse, Q D et al. Int J Gynecol Cancer. (2006) 16:1119-1129].

Radiation therapy in the form of EBRT and VBT with or without concurrent chemotherapy (chemoradiation) plays a major role in the treatment of cervical cancer both in the primary and adjuvant setting. It has been associated with major vaginal toxicity including stenosis, shortening, atrophy, fibrosis and dyspareunia [Id., citing Katz, A. et al. Int J Gynecol Cancer. (2001) 11:234-35; Schover, L R, et al., Cancer. (1989) 63:204-212; Bergmark, K. et al. N Engl J Med. (1999) 340(18):1383-89; Brand A H, et al. Int J Gynecol Cancer. (2006) 16(1):288-93; Bruner D W, et al. Int J Radiat Oncol Biol Phys. (1993) 27:825-830]. Primary or adjuvant radiation therapy has been associated with greater sexual dysfunction and vaginal toxicity compared to surgery alone. [Id., citing Greimel E R, et al. Psychoncology. (2009) 18(5):476-82, Frumovitz M, et al. J Clin Oncol. (2005) 23(30):7428-36]. Compared to age-matched controls, cervical cancer patients treated with radiation had significantly more sexual dysfunction and vaginal morbidity, including decreased libido (85%), dissatisfaction in sexual life (30%, reduced vaginal dimension (50%), dyspareunia (55%) and lack of lubrication (35%) [Id., citing Jensen P T, et al. Intl J. Radiation Oncology*Biology*Physics. (2003) 56(4):937-949]. The majority of patients with dyspareunia and lack of lubrication were distressed by their symptoms. [Id., citing Jensen P T, et al. Intl J. Radiation Oncology*Biology*Physics. (2003) 56(4):937-949].

### Ovarian Cancer

Primary treatment for ovarian cancer typically consists of a sequence of surgery and chemotherapy. Surgery involves hysterectomy, lymphadenectomy and tumor debulking with the goal of optimal cytoreduction before or after chemotherapy. Removal of the ovaries results in hormonal alterations that can cause adverse changes in sexual heath. [Id., at Hughes C, et al. Gynecologic Oncology. (1991) 40:42-45]. Menopausal symptoms triggered by cancer therapy can be more abrupt, prolonged and intense [Id., at Schover L R. J Clin Oncol. (2008) 26(5):753-8], and if not managed can lead to diminished quality of life, function and sexual desire. [Id., at Krychman M L, et al. Oncology (2006) 71(1-2):18-25]. Compared to healthy women, ovarian cancer survivors report increased vaginal dryness, more dyspareunia, less sexual activity and lower libido. [Id., at Liavaag A H, et al. Gynecol Oncol. (2008) 108(2):348-54, 54]. In one study, sexual function in ovarian cancer patients was investigated based on treatment modality, comparing surgery alone in early stage ovarian cancer patients (group 1), the combination of surgery and chemotherapy (group 2), and advanced inoperable or metastatic ovarian cancer patients receiving chemotherapy alone (group 3); Sexual satisfaction was decreased in all patients following treatment, but was more pronounced in groups 2 and 3 [Buković D, et al. (2008) 26(2):63-73].

### Vulvar Cancer

Treatment of vulvar cancer is based on the size, location and suspicion for lymph node metastases and consists of primary surgery with or without adjuvant radiotherapy or primary radiotherapy [Id., citing Stehman F B & Look K Y. Obstet Gynecol. (2006) 107(3):719-33]. Surgical treatment has evolved from a radical “en bloc” resection of the vulva with bilateral groin and pelvic lymph node dissection to a triple incision technique without pelvic lymphadenectomy [Id., citing Hacker, N F et al. Obstet Gynecol. (1981) 58:574-79, 61]. Despite changes in the surgical approach, sexual morbidity remains prevalent. Physical changes following surgery may include vaginal narrowing, numbness along the scar, removal of the clitoris, and change in tissue quality [Id., citing Barlow E L, et al. J Adv Nurs. (2014) 70(8):1856-66, Janda M, et al. Int J Gynecol Cancer. (2004) 14:875-88166, Weijmar Schulz W, et al. Cancer (1990) 66:402-407].

Radiation therapy has various roles in the treatment of vulvar cancer. In the adjunct setting, radiation therapy can be administered to the vulva to treat positive or close surgical margins and to the groins and pelvis in the setting of positive lymph nodes to prevent recurrence and improve survival. In advanced vulvar cancer not amenable to surgical resection, definitive chemoradiation is recommended.

Research evaluating sexual health following radiation for vulvar cancer patients is scarce. In a longitudinal study, a profound reduction in the ability to induce arousal and organism as well as a decrease in the perception of positive genital sensation was observed 6 months after surgery with or without adjuvant radiation, and did not improve during the 2 year follow-up [Id., citing Weijmar Schulz W, et al. Cancer (1990) 66:402-407].

### Genitourinary Syndrome (GSM) of Menopause

Genitourinary syndrome (GSM) of menopause is the accepted term to describe the genitourinary symptoms and signs related to menopause, such as dryness, burning, irritation and sexual symptoms such as discomfort or pain, and impaired sexual function [Alvisi, S. et al. Medicina (2019) 55 (10): 615]. This condition, which includes vulvovaginal atrophy (VVA), may also be accompanied by urinary signs and symptoms, such as urinary incontinence, painful urination (dysuria), slow and painful urination (stranguria), and frequent urinary tract infections [Id., citing Gandhi, J. et al. Am. J. Obstet. Gynecol. (2016) 215: 704-11]. It affects most peri- and postmenopausal women with a prevalence ranging from 36% to almost 90% according to most recent surveys. It has been reported that this condition is also already present in pre-menopausal years with a prevalence of 19% in women aged 40-45. [Id.] In spite of its high prevalence, it is still underdiagnosed and undertreated.

The drop in circulating hormone levels, especially estrogens, represents the main trigger determining vulvovaginal atrophy. The vaginal epithelia of post-menopausal women display flattened epithelial surfaces with features of keratinization and the absence of papillae. Multiple layers of parabasal cells with higher nucleus to cytoplasm ratio and few intermediate and superficial cells are present in which glycogen stores are reduced. This leads to a decrease in the number of Lactobacilli resulting in an increase in vaginal pH [Miller, E A et al. Front. Microbiol. (2016) 7: 1936]. The low percentage of Lactobacilli and the increase in the relative proportion of anaerobic bacteria found in post-menopausal women may predispose symptomatic VVA, although not all studies consistently report this association [Id., citing Hummelen, R. et al., PLoS ONE (2011) 6: e26602; Brotman, R M et al. Menopause (2014) 21: 450; Shen, J. et al. Sci. Rep. (2016) 6: 24380].

Hypoestrogenic vaginal states typically also include changes in the connective tissue composition with decreased type I/III collagen ratio, which leads to reduced tissue strength [Id., citing Hulmes, D J S. J. Struct. Biol. (2002) 137: 2-10]. Thinning of the vaginal epithelium increases susceptibility to trauma, resulting in bleeding, petechiae, and ulceration with any type of pressure including sexual activity or a simple gynecological maneuver. Thinning also exposes the underlying connective tissue, which is more vulnerable to inflammation or infection.

Due to these histological changes, clinical signs at the vaginal level include anything from dryness and insufficient hydration, redness, loss of elasticity, petechiae, ulceration, inflammation, atypical secretions, to fibrosis and vaginal obliteration. [Id.] The most frequent signs at a vulvar level include reduction in tissue thickness, labia agglutination, loss of pubic hair, and scratching lesions due to itching. Consequent symptoms include vaginal dryness and superficial dyspareunia with a prevalence of 78% and 76%, respectively [Id., citing Nappi, R E et al. Climacteric. (2016) 19: 188-97], which can be associated with itching, a burning sensation, and susceptibility to mechanical insults, leucorrhoea, or atypical secretions. At a vulvar level, the most frequent symptoms are burning, pain, increased susceptibility to physical and chemical irritants, and mechanical insults [Id., citing Murina, F. et al. Gynecol. Endocrinol. (2018) 34: 631-35].

Women's sexuality and relationships are greatly impacted by these changes [Id., citing Leiblum, S. et al. JAMA (1983) 249: 2195-98]. The REVIVE study suggested that VVA symptoms have a significant impact on the patients' ability to achieve pleasurable relations (74%) and spontaneity (70%). Seventy-five percent of sexually active post-menopausal women with VVA were reported to have a significantly reduced sex drive as a direct consequence of the symptoms related to this condition [Id., citing Nappi, R E et al. Climacteric. (2016) 19: 188-97]. A 2014 study showed that most women were worried that vaginal discomfort could have long-term effects on their relationship [Id., citing Simons, J A et al. Menopause (2014) 21: 137-42].

### Diabetes

Chronic hyperglycemia associated with diabetes can result in end organ dysfunction and failure which can involve the retina, kidneys, nerves, heart and blood vessels. [Tsalamandris, S. et al. Eur. Cardiol. (2019) 14 (1): 50-59, citing Inzucchi, S E. N. Eng. J. Med. (2013) 368: 193]

Diabetes mellitus is an important cause of sexual dysfunction both in men and women. In men, diabetic autonomic neuropathy, often reinforced by antihypertensive medication and vasculopathy, is a major cause of organic impotence, [Enzlin, P. et al. Diabetic Medicine (1998) 15: 809-15] In diabetic women, problems affecting the vaginal tract are very often neglected. Indeed, the literature regarding female sexual dysfunction and diabetes, is limited, perhaps because of a tendency to ignore sexual implications of physical illness in women, or because of the difficulties posed by the intricacies of the female sexual response. [Enzlin, P. et al. Diabetic Medicine (1998) 15: 809-15; Gupta, L. et al. Touch Medical Media (2018)].

Female sexual dysfunction is associated with both type 1 and type 2 diabetes. [Enzlin, P. et al. Diabetes Care (2009) 32 (5): 780-5], with the risk for sexual dysfunction more than 2.47 times greater for women with diabetes [Barnard, K D et al. Practical Diabetes (2019) 36.5]. For example, diabetic women are more predisposed to a high risk of recurrent vaginitis caused by Candida spp., leading to inflammation that results in dyspareunia and cystitis. These problems often are associated with vaginal dryness, redness, burning and itching during sexual intercourse [Carati, D. et al. Clin. Exp. Obstet. Gynecol. Doi: 10.12891/ceog3078.2016]. Candida albicans binds to epithelial cells more easily in diabetic women, and hyperglycemia can influence the humor al response, causing a reduction in neutrophils, chemotaxis and phagocytosis [Id., citing Segal, E. et al. J. Med. Vet. Mycol. (1984) 22: 191; Bagdade, J D et al., Diabetes (1974) 23: 9]. Vaginal dryness is twice as likely in diabetic women compared to non-diabetic women [Enzlin, P. et al., Diabetes Care (2002) 25(4): 672-77], citing Enzlin, P. et al. Diabet. Med. (1998) 15: 809-15].

The emerging role of inflammation in both type 1 and type 2 diabetes (T1D and T1D) pathophysiology and associated metabolic disorders has generated increasing interest in targeting inflammation to improve prevention and control of the disease [Tsalamandris, S. et al. Eur. Cardiol. (2019) 14 (1): 50-59].

### Treatment Options

Treatment options for women with conditions with sexual side effects run the gamut, from moisturizers to pharmaceuticals to energy-based devices. Despite the availability of many types of treatments, women remain unsatisfied with the choices for a variety of reasons, and alternative therapies with a moisturizer or a lubricant have not relieved the symptoms.

Various non-hormonal, non-prescription treatments exist for vaginal atrophy (VA), including, without limitation, increased coital activity, cessation of smoking, pelvic-floor physiotherapy (PT), and lubricants or moisturizers [Alvisi, S. et al. Medicina (2019) 55 (10): 615], citing Leiblum, S. et al. JAMA (1983) 249: 2195-98]. Many women use over-the-counter (OTC) products such as vaginal lubricants and moisturizers. International guidelines consider these to be the first line of therapy in the treatment of VVA being free from significant contraindications and side effects [Id., citing Stuenkel, C A et al. Clin. Endocrinol. Metab. (2015) 100: 3975-4011]. They can be used alone or in combination with hormonal therapies as needed. This treatment option is also recommended for women for whom the use of vaginal estrogen preparations is unacceptable. The osmolality, pH, and the composition of these products, either lubricants or moisturizers, needs to be similar to vaginal secretion [Id., citing Edwards, D., Panay, N. Climacteric. (2016) 19: 151-61].

The main difference between vaginal lubricants and moisturizers is the timing of application. Vaginal lubricants are particularly indicated for women whose main concern is vaginal dryness during intercourse. Lubricants provide short-term relief from dryness and reduce difficult or painful sexual intercourse (dyspareunia). They can be water-based, which are water-soluble and have a tendency to dry out; oil based, which are more durable, but with a lower lubricating effect; or silicone-based. Some lubricants contain glycerin, propylene glycol, sweeteners and parabens, which may have an impact on the pH and osmolality of water-based products [Id., citing Edwards, D., Panay, N. Climacteric. (2016) 19: 151-61].

Vaginal moisturizers are insoluble hydrophilic cross-linked polymers with a characteristic bio-adhesiveness that is able to adhere to the epithelium of the vaginal wall by retaining water. They can also contain a large amount of excipients that influence the pH and the osmolality of the formulation. They can be used more regularly, rather than just in association with sexual activity, and have a longer lasting effect, improving the moisture of the vaginal mucosa and reducing the pH. The frequency of use is directly proportional to the severity of VVA [Id., citing Edwards, D., Panay, N. Climacteric. (2016) 19: 151-61]. Application in the acute phase consists in local applications in the evening, before going to bed, for seven to ten consecutive days, so that they can act throughout the night, followed by two local applications per week to maintain the beneficial effects. The most commonly used moisturizers are based on hyaluronic acid (HA), a glycosaminoglycan produced by fibroblasts, which is the main component of the extracellular matrix. The possible action mechanism of hyaluronic acid is cell migration because it has a very high capacity to bind water, which may facilitate cellular movement [Salwowska, N M K J. Cosmet. Dermatol. (2016) 15: 520-26]. In the case of tissue damage, it has been suggested that HA may stimulate the migration and proliferation of fibroblasts and therefore the deposition of collagen fibers, in addition to stimulating neo-angiogenesis and re-epithelialization. If used on a regular basis, daily or every 2-3 days, HA based products improve symptoms of vaginal dryness, with an effect that has been compared with the effect of topical estrogen therapy [Id., citing Mitchell, C M et al. Menopause (2019) 26: 816-22]. Some adverse effects have been reported with the use of HA [Id., citing Chung, K L et al. Aesthet. Surg. J. (2019) doi: 10.1093/asj/sjz222]; most have occurred after injections. They include local reactions namely bruising, erythema, swelling, and, rarely, more severe events such as tissue necrosis, infection, or pulmonary complications. To the best of our knowledge, no severe adverse effects have been reported with the use of HA-based vaginal moisturizers. [Id.]

Other possible components of vaginal moisturizers are ozonides, intermediate products of ozone, which act as a biological reservoir preserving the therapeutic power of the molecule. In contact with biological tissue, ozonides activate quickly, stimulating the local microcirculation to induce neo-angiogenesis, promoting tissue repair, and inhibiting pro-inflammatory prostaglandins [Id., citing DiMauro, R. et al. Int. J. Mol. Sci. (2019) 20: 634].

Oral vitamin D and vaginal vitamin E have been proposed for the treatment of VVA, but efficacy data are limited and sometimes discordant. Vitamin D stimulates the proliferation of the vaginal epithelium by activating the vitamin D receptor (VDR). Vaginal vitamin E is involved in the metabolism of all cells and prevents tissue damage caused by oxidants. This facilitates blood circulation, which consequently increases the metabolism of vaginal connective tissues and enhances the moisture and flexibility of vaginal walls [Id., citing Yildirim, B. et al. Maturitas (2004) 49: 334; Pitsouni, E. et al. Eur. J. Obstet. Gynecol. Reprod. Bio. (2018) 229: 45-56; Costantino, D. & Guaraldi, C. Eur. Rev. Med. Pharmacol. Sci. (2008) 12: 411].

Oral and vaginal probiotics for improving vaginal microbiota may be beneficial for the treatment of VVA symptoms, however, placebo-controlled trials that prove their effectiveness are lacking [Id., citing Muhleisen, A L & Herbst-Kralovetz, M M. Maturitas (2016) 91: 42-50].

Oral phytoestrogens are not effective [Id., citing Grant, M D et al. AHRQ Comparative Effectiveness Reviews. Agency for Healthcare Research and Quality (US); Rockville, Md., USA: 2015. Menopausal symptoms: Comparative. Effectiveness of therapies.] while preliminary investigations suggest that topical phytoestrogens may have a beneficial effect on VVA, improving genital symptoms, maturation index, vaginal pH, morphology, and expression of estrogen receptors in the vaginal epithelium [Id., citing Kagan, R. et al. Menopause (2010) 17: 281-89].

Hormone treatments of menopause (HTM), meaning the association of estrogen-progestins, estrogen-bazedoxifene, tibolone, or exclusively estrogens in hysterectomized women, have a beneficial effect on many symptoms related to menopause including VVA. According to international guidelines, they are not recommended in women who suffer only from vaginal and vulvar symptoms, however, when they are used for primary indications, the evidence shows that HTM are able to restore the physiological vaginal pH, the maturation index, and the thickness of the vaginal epithelium, its vascularization and lubrication [Id., citing The NAMS 2017 Hormone Therapy Position Statement Advisory Panel The 2017 hormone therapy position statement of the North American Menopause Society. Menopause. 2017; 24:728-753. doi: 10.1097/GME.0000000000000921].

International guidelines recommend local hormonal therapy as a second step in the event of the ineffectiveness of vaginal lubricants and moisturizers [Id., citing The NAMS 2017 Hormone Therapy Position Statement Advisory Panel The 2017 hormone therapy position statement of the North American Menopause Society. Menopause. 2017; 24:728-753. doi: 10.1097/GME.0000000000000921]. Options available include estradiol, estriol, conjugated estrogens or promestriene gels, creams, ovules, tablets, or rings. These are specifically indicated for the treatment of VVA including dyspareunia. All estrogen-based vaginal products are more effective than a placebo for VVA. Vaginal estrogens are superior to lubricants and moisturizers in studies lasting at least six to twelve months [Id., citing Lethaby, A. et al. Cochrane Database. Syst. Rev. 2016; 8:CD001500. doi: 10.1002/14651858.CD001500.pub3; Jokar, A. et al. Intl J. Community Based Nurs. Midwifery (2016) 4: 69-78]. The recommended dose is commonly a local daily application for two weeks as an attack therapy, and then application twice a week as maintenance therapy [Id., citing Salwowska, N M, et al. J. Cosmet. Dermatol. (2016) 15: 520-26].

Ospemifene is the only selective estrogen receptor modulator (SERM) to be indicated for the treatment of VVA. It has been approved by the U.S. Food and Drug Administration (FDA) for the treatment of moderate to severe dyspareunia and by the European Medical Agency (EMA) for the treatment of moderate to severe VVA in women, with or without a uterus, who are not candidates for local estrogen therapy [Id., citing De Gregorio, M W, et al. Steroids (2014) 90: 82-93]. It exerts a positive effect on the vaginal epithelium while having, at the same time, a neutral or minimal effect on the other estrogen-dependent organs. In particular, it seems to have a neutral effect on the endometrium and the cardiovascular system, and an anti-estrogenic effect in pre-clinical studies on the breast. It is used at a dose of 60 mg daily. The effects on the signs of VVA are visible after four weeks of treatment such as the increase of superficial cells, the reduction of basal cells, and the reduction of vaginal pH [Id., citing Alvisi, S. et al. Gynecol. Endocrinol. (2017) 33: 946-50]. A significant effect on symptoms such as dryness and dyspareunia has been demonstrated to occur after 12 weeks of treatment [Id., citing Goldstein, S R et al. Climacteric. (2014) 17: 173-82]. The efficacy of ospemifene at a histological level in both vaginal and vulvar tissue has been demonstrated by observing increases in vaginal and vulvar epithelial thickness, glycogen content, proliferation index, and an increase in vaginal estrogen receptor alpha (ERα) [Id., citing Alvisi, S. et al., Gynecol. Endocrinol. (2017) 33: 946-50; Alvisi, S. et al. J. Sex. Med. (2018) 15: 1776-1784]. Ospemifene has also been shown to improve atrophy of the vulvar vestibule and to normalize vestibular sensitivity by increasing the perception threshold at a vulvar level [Id., citing Goldstein, S W et al. Sex. Med. (2018) 6: 154-61]. In a short-term study, it has also been shown to increase ratio type I and type III collagen at the vaginal level, suggesting possible beneficial long-term effects on vaginal connective tissue [Id., citing Alvisi, S. et al. J. Sex Med. (2018) 15: 1776-84].

Prasterone (dehydroepiandrosterone) has been introduced to the market for the treatment of VVA. It acts as a precursor of intracellular sex steroid androgens and estrogens. Since the conversion happens inside the cells, serum estradiol remains within the normal values for postmenopausal women, thereby probably avoiding the risk of systemic effects [Id., citing Martel, C. et al. J. Steroid Biochem. Mol. Biol. (2016) 159: 142-53]. The efficacy of dehydroepiandrosterone (DHEA) has been demonstrated in a prospective, randomized, double-blind, placebo-controlled phase III clinical trial that examined the effects of daily intravaginal prasterone (6.5 mg) on four co-primary objectives, namely, the percentage of vaginal parabasal cells, percentage of vaginal superficial cells, vaginal pH, and moderate to severe dyspareunia, identified by women as the most bothersome VVA symptom. It may also be effective on the reduction of libido with a possible action on nerve endings, however, more scientific evidence is needed on this aspect [Id., citing Labrie, F. et al. Menopause (2018) 25: 1339-53]. The endometrium is not affected by DHEA because the enzymes required to transform DHEA into estrogens are absent in the endometrium. Although no systemic increase of estrogen level has been reported, a history of breast cancer remains a contraindication.

Energy-based devices may have some potential benefits in treating patients with VVA. [Id., citing ACOG Position Statement. Fractional Laser Treatment of Vulvovaginal Atrophy and U.S. Food and Drug Administration Clearance. The American College of Obstetricians and Gynecologists; Washington, D.C., USA: 2016].

For example, laser or radiofrequency waves act by heating the connective tissue of the vaginal wall to 40° C. to 42° C. In this way, they allegedly induce collagen contraction, neocollagenesis, vascularization, and growth factor infiltration that ultimately revitalizes and restores the elasticity and moisture of the vaginal mucosa. The proposed mechanism is the activation of heat shock proteins and tissue growth factors to stimulate new collagen synthesis and epithelial remodeling [Id., citing Salvatore, S. et al. Curr. Opin. Obstet. Gynecol. (2015) 27: 504-8].

The efficacy of laser therapy in the treatment of VVA has been suggested by the improvement of GSM symptoms, VHI scores, and female sexual function index (FSFI) in many studies with its effectiveness at least as good as that of local estrogen based treatments [Id., citing Salvatore, S. et al. Climacteric. (2015) 18: 219-225; Gambacciani, M. et al. Climateric. (2018) 21: 148-52]. However, none of these studies were either sham or placebo controlled and the lack of sufficient information, especially concerning long-term safety, prompted the FDA in 2018 to warn against the indiscriminate marketing of laser treatments [Id., citing Food and Drug Administration FDA Warns Against Use of Energy-Based Devices to Perform Vaginal ‘Rejuvenation’ or Vaginal Cosmetic Procedures: FDA Safety Communication]. Although authors generally suggest that the procedure is well tolerated, being rapid and painless, increased vaginal pain, scarring, fibrosis, and vaginal wall lacerations have been reported [Id., citing Gordon, C. et al. Menopause (2019) 26: 423-27].

Radiofrequency devices most commonly used by gynecologists are the transcutaneous temperature-controlled radiofrequency (TTCRF), and more recently, the low-energy dynamic quadripolar radiofrequency (DQRF). The mechanism of treatment is to trigger anatomical remodeling in the vaginal and vulvar tissues. There have been some small studies that prove its effectiveness on vaginal symptoms, sexual function as well as urinary symptoms, but again they have been small, non-randomized studies [Id., citing Caruth, J C. Surg. Technol. Int. (2018) 32: 145-49; Vicariotto, F. & Raichi, M. Minerva Ginecol. (2016) 68: 225-36; Vicariotto, F. et al. Minerva Ginecol. (2017) 69: 342-49].

The use of vaginal dilators to prevent vaginal stenosis from pelvic radiation is often recommended to gynecologic cancer patients, but data to support its effectiveness is conflicting [Huffman, L B et al. Gynceol. Oncol. (2016) 140(2): 359-68, citing [Miles, T., Johnson, N. Cochrane Database of Systematic Reviews (2014) 9: CD007291; Johnson, N. et al., BJOG (2010) 117 (5): 522-31] and adherence to dilator use is poor. [Id., citing Friedman, L C et al. Int. J. Radiat. Oncol. Biol. Phys. (2011) 80 (3): 751-57]. Dilation involves inserting and rotating a phallus-shaped appliance in the vagina approximately three times a week for about 5 minutes to stretch the skin. While dilation might separate the adhesions formed by the denuded epithelium, thus possibly preventing stenosis. [Id., citing Faithfull, S. & Wells, M. Supportive Care in Radiotherapy. Edinburgh: Churchill Livingstone (2003); Hassey-Dow, K. Nursing care in Radiation Oncology; Philadelphia: WB Saunders (1992); Krumm, S. J. Psychosomatic Obstetric Gynaecology (1993) 14 (1): 51-63; Rice A. Intl J. Palliative Nursing (2000) 6(80): 392-97, it is also plausible that stretching the vagina during the inflammatory phase of radiotherapy treatment might cause additional scarring and promote additional damage, both physically and psychologically.

### Options for Treatment of Breast Cancer Survivors

In the case of women with previous or ongoing breast cancer, the options for treatment are unfortunately limited. All hormone-based therapies are contraindicated including vaginal isoflavone-based soy therapies, as there have been no studies on their safety in this cohort of women. Non-hormonal approaches are the first-line choices during or after breast cancer [Id., citing American College of Obstetricians and Gynecologists' Committee on Gynecologic Practice. Farrell R. ACOG. COMMITTEE OPINION No. 659. The Use of Vaginal Estrogen in Women with a History of Estrogen-Dependent Breast Cancer. Obstet. Gynecol. 2016; 127:e93-e96]. The options therefore are to offer these women moisturizers and vaginal lubricants, laser or radiofrequency treatments.

The described invention provides a soothing, less offensive alternative.

### SUMMARY OF THE INVENTION

According to one aspect, the present disclosure provides a cosmetic composition formulated for topical application comprising: water-based gel component comprising hyaluronic acid; a botanical ingredient component comprising decarboxylated cannabidiol (CBD) representing about 20% of an isolate, and a cosmetic composition stabilizing system comprising arginine, p-anisic acid, and levulinic acid, wherein the composition is a slightly viscous non-occluded water based liquid; pH of the finished product ranges from about 4.0 to about 5.0 inclusive; and the composition is not psychoactive.

According to some embodiments of the cosmetic composition, the hyaluronic acid comprises about 0.10 to about 0.50 wt %, inclusive, low molecular weight hyaluronic acid (LMWHA) and about 0.50 to about 1.50 wt %, inclusive, high molecular weight hyaluronic acid (HMWHA), wherein a ratio of the HMWHA to MMWHA ranges from 1:0.07-to 1:1, inclusive. According to some embodiments, molecular weight of the HMWHA ranges from 1,000-1,800 kDa, inclusive. According to some embodiments, the molecular weight of the HMWHA is at least 1000, 1100, 1200, 1300, 1400, 1500, 1600, 1700, or 1800 kDa. According to some embodiments, molecular weight of the LMWHA is at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, or at least 9 kDa to less than 10 kDa. According to some embodiments, the decarboxylated CBD representing 20% of an isolate is in form of a THC free nano-infused water soluble powder. According to some embodiments, the cosmetic stabilizing system comprises about 0.25 wt % to about 1.00 wt %, inclusive, arginine levulinate and about 0.05 wt % to about 0.50 wt %, inclusive arginine anisate. According to some embodiments, viscosity of the composition ranges from 5000 to 7500 centipoise, inclusive at room temperature. According to some embodiments, the composition comprises about 1.0 wt % to about 5.0 wt %, inclusive of the THC-free nanoinfused water-soluble powder comprising about 20% decarboxylated CBD.

According to another aspect, the present disclosure provides a method for promoting and maintaining vaginovulval tissue vitality and tissue health in a female subject in need thereof comprising administering topically to the subject a cosmetic composition comprising: a water-based gel component comprising hyaluronic acid; a botanical ingredient component comprising a decarboxylated cannabidiol (CBD) representing 20% w/w of an isolate; and a cosmetic composition stabilizing system comprising arginine, p-anisic acid, and levulinic acid, wherein the composition is a slightly viscous non-occluded water based liquid; pH of the composition ranges from about 4.0 to about 5.0, inclusive; the composition is not psychoactive; therapeutic effects of the water-based gel component and the botanical ingredient component may be complementary; and the composition promotes and maintains vaginovulval tissue vitality and tissue health.

According to some embodiments of the method, the female subject in need thereof is a female subject susceptible to or experiencing vaginovulval symptoms of trauma, insult or injury or a female subject experiencing genitourinary symptoms of trauma, insult or injury. According to some embodiments of the method, the female subject in need thereof is a menopausal subject; or the female subject in need thereof is a diabetic subject; or the female subject in need thereof is a subject that will undergo, is undergoing or has undergone treatment comprising radiation therapy to treat a gynecologic cancer; or the female subject in need thereof is a breast cancer survivor. According to some embodiments, the gynecologic cancer is an endometrial cancer, a cervical cancer; an ovarian cancer; or a vulvar cancer. According to some embodiments, parameters of vaginovulval tissue vitality include one or more of improved tissue strength, appropriate vaginal pH; reduced susceptibility to trauma/mechanical insult, reduced inflammation, reduced itching, improved wound healing, and improved tissue elasticity. According to some embodiments, the cosmetic composition is effective to restore wounded tissue to a healthy tissue. According to some embodiments, the composition, compared to an untreated control: modulates vaginal pH; or improves healing and rejuvenation of wounded tissue; or reduces susceptibility to trauma or mechanical injury; or reduces symptoms of trauma, insult or injury; or reduces clinical signs of dryness and insufficient hydration (e.g., loss of elasticity, inflammation); or reduces itching; or a combination thereof. According to some embodiments, improving healing and rejuvenation of wounded tissue comprises improving tissue strength. According to some embodiments, symptoms of trauma, insult or injury comprise one or more of dryness, burning, irritation, discomfort or pain. According to some embodiments, clinical signs of dryness and insufficient hydration include loss of elasticity, inflammation or both. According to some embodiments, the hyaluronic acid comprises about 0.10 wt % to about 0.50 wt %, inclusive, low molecular weight hyaluronic acid (LMWHA) and about 0.50 wt % to about 1.50 wt %, inclusive, high molecular weight hyaluronic acid (HMWHA), wherein ratio of the HMWHA to LMWHA ranges from 1:0.7 to 1:1, inclusive. According to some embodiments, molecular weight of the HMWHA ranges from 1,000-1,800 kDa, inclusive. According to some embodiments, the molecular weight of the HMWHA is at least 1000, 1100, 1200, 1300, 1400, 1500, 1600, 1700, or 1800 kDa. According to some embodiments, molecular weight of the LMWHA is at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, or at least 9 kDa to less than 10 kDa. According to some embodiments, the decarboxylated CBD is in form of a THC free nano-infused water soluble powder. According to some embodiments, the cosmetic stabilizing system comprises about 0.25 wt % to about 1.00 wt %, inclusive, arginine levulinate and about 0.05 wt % to about 0.50 wt %, inclusive arginine anisate. According to some embodiments, viscosity of the composition ranges from 5000 to 7500 centipoise, inclusive at room temperature. According to some embodiments, the composition comprises about 1.0% to about 5.0%, inclusive of the THC-free nanoinfused water-soluble powder comprising about 20% decarboxylated CBD.

According to another aspect, the present disclosure provides a method for promoting and maintaining perianal tissue vitality and tissue health in a subject with a hemorrhoidal disease, the method comprising administering topically to the subject a cosmetic composition comprising: a water-based gel component comprising hyaluronic acid; a botanical ingredient component comprising a decarboxylated cannabidiol (CBD) representing 20% w/w of an isolate; and a cosmetic composition stabilizing system comprising arginine, p-anisic acid, and levulinic acid, wherein the composition is a slightly viscous non-occluded water based liquid; pH of the composition ranges from about 4.0 to about 5.0, inclusive; the composition is not psychoactive; therapeutic effects of the water-based gel component and the botanical ingredient component may be complementary; and the composition reduces one or more cutaneous symptoms of the hemorrhoidal disease.

According to some embodiments of the method, the hemorrhoidal disease comprises external hemorrhoidal tissue. According to some embodiments, the composition, compared to an untreated control: improves healing and rejuvenation of the external hemorrhoidal tissue; or reduces susceptibility of the external hemorrhoidal tissue to trauma or mechanical injury; or reduces symptoms of trauma, insult or injury; or reduces clinical signs of dryness and insufficient hydration of the external hemorrhoidal tissue; or reduces itching; or a combination thereof. According to some embodiments, improving healing and rejuvenation of the external hemorrhoidal tissue comprises improving tissue strength. According to some embodiments, symptoms of trauma, insult or injury comprise one or more of dryness, burning, irritation, discomfort or pain. According to some embodiments, clinical signs of dryness and insufficient hydration include loss of elasticity, inflammation or both. According to some embodiments, the hyaluronic acid comprises about 0.10 wt % to about 0.50 wt %, inclusive, low molecular weight hyaluronic acid (LMWHA) and about 0.50 wt % to about 1.50 wt %, inclusive, high molecular weight hyaluronic acid (HMWHA), wherein ratio of the HMWHA to LMWHA ranges from 1:0.7 to 1:1, inclusive. According to some embodiments, molecular weight of the HMWHA ranges from 1,000-1,800 kDa, inclusive. According to some embodiments, the molecular weight of the HMWHA is at least 1000, 1100, 1200, 1300, 1400, 1500, 1600, 1700, or 1800 kDa. According to some embodiments, molecular weight of the LMWHA is at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, or at least 9 kDa to less than 10 kDa. According to some embodiments, the decarboxylated CBD is in form of a THC free nano-infused water soluble powder. According to some embodiments, the cosmetic stabilizing system comprises about 0.25 wt % to about 1.00 wt %, inclusive, arginine levulinate and about 0.05 wt % to about 0.50 wt %, inclusive arginine anisate. According to some embodiments, viscosity of the composition ranges from 5000 to 7500 centipoise, inclusive at room temperature. According to some embodiments, the composition comprises about 1.0% to about 5.0%, inclusive of the THC-free nano-infused water-soluble powder comprising about 20% decarboxylated CBD.

### BRIEF DESCRIPTION OF THE DRAWINGS

FIG. 1 presents a diagram of skin anatomy. Taken from Stedman's Medical Dictionary, 27th Ed., Lippincott, Williams & Wilkins, Baltimore, Md. (2000), at 1647.

FIG. 2 illustrates layers of the epidermis from the stratum corneum to the dermis.

FIG. 3A-FIG. 3C show micrographs of a L929 fibroblast cytotoxicity test. FIG. 3A, complete medium (control); FIG. 3B, complete medium plus miniHA™; FIG. 3C, complete medium+HA-Oligo. No morphological cytotoxicity was observed.

FIG. 4 is a plot of relative growth rate (RGR) versus concentration of HA oligosaccharides (%, w/v) added to in vitro cultures of L929 fibroblasts.

FIG. 5A and FIG. 5B show results of moisture retention experiments with HA. FIG. 5A depicts bar graphs showing moisture retention capacity of low molecular weight hyaluronic acid miniHA™ (molecular weight 8300 Da) as measured by corneometer before topical application of the sample, and at 1 h, 2 h, 3 h, 4 h, 6 h and 8 h after application. FIG. 5B depicts bar graphs showing moisture retention capacity of high molecular weight hyaluronic acid (molecular weight 1,170 kDa) as measured by corneometer before topical application of the sample, and at 1 h, 2 h, 3 h, 4 h, 6 h and 8 h after application.

FIG. 6 is a graph of corneometer value (%) vs. time for 0.1% mini HA, 0.2% miniHA and 0.5% HA. As shown, the higher concentration of miniHA, i.e., 0.5% miniHA™, has a better moisture retention capacity than either 0.1% miniHA or 0.2% miniHA.

FIG. 7 is a graph of corneometer value (%) vs. time for 0.2% HA (molecular weight 1,630,000 DA); 0.2% miniHA™ (molecular weight 8,300); and 0.1% HA+0.1% miniHA. As shown, when miniHA™ was used together with HMWHA, the moisturizing effect was better greater than for each alone

FIG. 8A and FIG. 8B show results of the moisture retention test (FIG. 8A) and the TEWL test (FIG. 8B). FIG. 8A is a graph of corneometer value (%) vs. time for 0.1% mini HA, 0.1% HA-1630 kDa, and for 0.1% HA-270 kDa. As shown, the lower the molecular weight of HA, the better the moisture retention was. The miniHA group had the highest value of skin hydration at each time point. FIG. 8B is a graph of transepidermal water loss (TEWL) versus time for 0.1% mini HA, 0.1% HA-1630 kDa, and for 0.1% HA-270 kDa. The data show that the higher molecular weight of HA, the less the skin water was reduced.

FIG. 9A and FIG. 9B show results of the moisture retention test (FIG. 9A) and the TEWL test (FIG. 9B). FIG. 9A shows bar graphs of corneometer value (%) vs. time for 0.1% mini HA+0.1% HA-270 kDa. FIG. 9B shows bar graphs of transepidermal water loss (TEWL) versus time for 0.1% mini HA+0.1% HA-270 kDa. Together, the moisturizing effect is better than either ingredient alone.

FIG. 10 is a cross-section through human reconstructed epidermis.

FIG. 11 is a cross-section through human reconstructed full thickness sin.

FIG. 12 is a schematic of the experimental system. The product applied on the surface penetrates the human reconstructed tissue; a certain amount of the product is maintained by the tissue structure.

FIG. 13 is a graph of % absorption vs. time for reconstructed epidermis; full thickness reconstructed skin; and dermis. The dermis results were mathematically calculated by subtraction of the full thickness and epidermis absorption data.

FIG. 14 is a microscopic image of human heratinocytes (HaCaT line) in culture used in the study in Example 4.

FIG. 15 is a bar graph showing relative cell viability (OD 550 nm) versus RS-0198A at concentrations 3%, 1%, 0.3%, 0.1%, 0.03%, 0.01%, 0.003%, and 0.001%, compared to the untreated control group. **** represents statistical significance with p-value <0.0001. * represents statistical significance with p-value <0.05.

FIG. 16 is a bar graph showing relative cell viability (OD 550 nm) versus RS-0198B at concentrations 3%, 1%, 0.3%, 0.1%, 0.03%, 0.01%, 0.003%, and 0.001%, compared to the untreated control group. **** represents statistical significance with p-value <0.0001. ** represents statistical significance with p-value <0.01.

FIG. 17 is a bar graph showing relative cell viability (OD 550 nm) versus RS012221 at concentrations 3%, 1%, 0.3%, 0.1%, 0.03%, 0.01%, 0.003%, and 0.001%, compared to the untreated control group. **** represents statistical significance with p-value <0.0001.

FIG. 18 is a bar graph representing wound area relative to an untreated control after treatment for 24 hours of human keratinocytes (HaCaT) with RS-0198A at 0.00001%, 0.0001%, 0.001% and RS-0198B at 0.0001%, 0.001%, and 0.01%. Human EGF at 20 ng/ml was included as a positive control. * Represents statistical significance with p-value <0.05. ** Represents statistical significance with p-value <0.01.

FIG. 19 is a bar graph representing wound area relative to an untreated control after treatment for 24 hours of human keratinocytes (HaCaT) with RS012221 at 0.001% and 0.01% concentrations. Human EGF at 20 ng/ml was included as a positive control. ** Represents statistical significance with p-value <0.01. **** Represents statistical significance with p-value <0.0001.

FIG. 20 is a bar graph representing wound healing relative to an untreated control after treatment for 24 hours of human keratinocytes (HaCaT) with RS-0198A at 0.00001%, 0.0001%, and 0.001% and RS-0198B at 0.0001%, 0.001%, and 0.01%. Human EGF at 20 ng/ml was included as a positive control. * Represents statistical significance with p-value <0.05. ** Represents statistical significance with p-value <0.01.

FIG. 21 is a bar graph representing wound healing relative to an untreated control after treatment for 24 hours of human keratinocytes (HaCaT) with RS012221 at 0.001% and 0.01% concentrations. Human EGF at 20 ng/ml was included as a positive control. ** Represents statistical significance with p-value <0.01. **** Represents statistical significance with p-value <0.0001.

FIG. 22A-FIG. 22P show microscopic images of wound healing from scratches performed on human keratinocyts (HaCaT) monolayers immediately before (0 h) and 24 h after treatment. Human EGF at 20 ng/ml was included as a positive control. FIG. 22A Control, t=0 h; FIG. 22B Control, t=24 hr; FIG. 22C, EGF t=0 h; FIG. 22D, EGH t=24 h; FIG. 22E RS-0198A, 0.00001%, t=0 h; FIG. 22F, RS-0198A, 0.00001%, t=24 h; FIG. 22G, RS-0198A, 0.0001%, t=0 h; FIG. 22H RS-0198A, 0.0001%, t=24 h; FIG. 22I, RS-0198A, 0.001%, t=0 h; FIG. 22J, RS-0198A, 0.001%, t=24 h; FIG. 22K, RS-0198B, 0.0001%, t=0 h; FIG. 22L, RS-0198B, 0.0001%, t=24 h; FIG. 22M, RS-0198B, 0.001%, t=0 h; FIG. 22N, RS-0198B, 0.001%, t=24 h; FIG. 22O, RS-0198B, 0.01%; t=0 h; FIG. 22P, RS-0198B, 0.01%, T=24 h.

FIG. 23A-FIG. 23H show microscopic images of wound healing from scratches performed on human keratinocyts (HaCaT) monolayers immediately before (0 h) and 24 h after treatment. Human EGF at 20 ng/ml was included as a positive control. FIG. 23A, Control, 0.5%, t=0 h; FIG. 23B, Control, 0.5%, t=24 h; FIG. 23C EGF, 20 ng/ml, t=0 h; FIG. 23D, EGF, 20 ng/ml, t=24 h; FIG. 23E, RS012221, 0.01%, t=0 h; FIG. 23F, RS12221, 0.01%, t=24 h; FIG. 23G RS012221, 0.001, t=0 h; FIG. 23H, RS012221, 0.001%, t=24 h.

### DETAILED DESCRIPTION OF THE INVENTION

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 20% of the numerical value of the number with which it is being used. Therefore, about 50% means in the range of 40%-60%.

The term “active agent” as used herein refers to the ingredient, component or constituent of a composition responsible for the intended cosmetic effect.

The term “administer” as used herein means to give or to apply. The term “administering” as used herein includes in vivo administration, as well as administration directly to tissue ex vivo.

The term “analog” as used herein refers to a compound whose structure is related to that of another compound but whose chemical and biological properties may be quite different. A “direct analog” possesses chemical and pharmacological similarities to an existing compound; a “structural analog” possesses structural similarities only; it can differ in one or more atoms, functional groups or substructures and have different physical, chemical, biochemical or pharmacological properties. The term “functional analog” as used herein refers to chemically different compounds displaying similar pharmacological properties.

The term “angiogenesis” as used herein refers to the formulation of new blood vessels from preexisting functional vessels. Under physiological conditions, angiogenesis depends on the balance of positive and negative angiogenic modulators within the vascular microenvironment and requires the functional activities of a number of molecules, including angiogenic factors, extracellular matrix proteins, adhesion receptors, and proteolytic enzymes. Normally, the turnover of endothelial cells is very low, due to a balance of stimulators and inhibitors. However, under certain conditions (e.g. low pO2, low pH, hypoglycemia, mechanical stress, injury, immune/inflammatory stimuli, tumors) there is a dramatic increase in endothelial proliferation—a phenomenon termed activation of “the angiogenic switch.” Under these circumstances, the influence of the activators exceeds those of the inhibitors. These regulators of angiogenesis include growth factors, proteases and protease inhibitors, cytokines and chemokines, and a miscellaneous catch-all category termed “endogenous modulators” Examples of modulators of angiogenesis are listed in Table 1 [Gerritsen, M E. Chapter 8—Angiogenesis, In Handbook of Physiology, Microcirculation, 2d Ed. Elsevier, Inc. (2008), Tuma, R F, et al Eds, pp. 351-83)

TABLE 1
Examples of modulators of angiogenesis.

| Activator | Inhibitor |
|---|---|
| Angiogenin | Angioarrestin |
| Angiopoietins | Angiostatin |
| Del-1 | Anti-angiogenic antithrombin III |
| Endocrine Gland-VEGF | Arrestin |
| Fibroblast growth factors | Canstatin |
| Follistatin | Endostatin |
| Hepatocyte growth factors | Gro-beta |
| Leptin | Interferon alpha |
| Midkine | Interleukin 12 |
| Platelet-derived growth factor | Pigment epithelium-derived factor |
| Platelet-derived endothelial growth factor | Platelet Factor 4 Stannicalcin-1 |
| | Thrombospondin |
| Pleiotropin | Tissue inhibitor of metalloproteases |
| Proliferin | Tumstatin |
| Transforming growth factor | tRNA synthase |
| Vascular endothelial growth factors | Vasculostatin |
| Activator | Inhibitor |
| | Vasohibin |
| | Vascular endothelial growth inhibitor |
| | Vascular endothelial statin |

Hypoxia is one of the major drivers of angiogenesis. Hypoxia-inducible factor 1 (HIF) is a heterodimer of two DNA binding proteins, HIF-1α and HIF-1β (also known as aryl hydrocarbon nuclear translocator, ARNT). Under normal oxygen tensions, HIF-la undergoes post-translational modification (by prolyl hydroxylases) leading to the recognition of HIF-la by an ubiquitin ligase (von Hippel-Lindau protein). Ubiquitinated HIF-1a is then targeted for destruction by the proteasome. In contrast, during hypoxia, the activity of the prolyl hydroxylases is reduced and HIF-1α is stabilized, allowing it to form heterodimers with HIF-1β. The HIF heterodimers bind to specific hypoxia response elements, thereby leading to the upregulation of expression of a number of hypoxia-induced genes (e.g., adrenomedullin, angiopoietin-2, cyclooxygenase-2, endothelin-1, endothelin-2, hepatocyte growth factor, interleukin 8, leptin, migration inhibitor factor, monocyte chemotactic protein-1, nitric oxide synthase, placentaql growth factor, plasminogen activator inhibitor-1, stanniocalcin 1, stromal cell-derived factor 1 (CXCL12), TGFα, TGF-β1, TGF-β3, Tie-1, Urokinase receptor, VEGF-A, VEGFR-1), many of which are known to play important roles in angiogenesis. One of the most important genes upregulated by hypoxia-inducible factor 1 (HIF) is VEGF-A. Once hypoxia sets up a VEGFA source, endothelial cells will migrate toward the gradient of VEGFA that is sensed by the VEGFR2 receptor. Iruela-Arispe, M. and Zovein, A. In Fetal and Neonatal Physiology 5^{th} Ed. (2017) Elsevier, Inc.) Vol. 1: 85-89.e2,

The term “anorectal” as used herein refers to relating to both anus and rectum. The term “anus” or “anal orifice” as used herein refers to the lower opening of the digestive tract, lying in the cleft between the buttocks, through which fecal matter is extruded. The term “rectal” as used herein means relating to the rectum, meaning the terminal portion of the digestive tube, extending from the rectosigmoid junction to the anal canal. The term “anal canal” as used herein refers to the most terminal part of the lower GI tract/large intestine, which lies between the anal verge (anal orifice, anus) in the perineum below and the rectum above.

The term “anti-inflammatory” as used herein refers to reducing inflammation (redness, swelling, and pain) in the body by inhibiting inflammatory mediators in the body that cause inflammation.

The term “anti-irritant” as used herein refers to preventing or reducing soreness, roughness, or inflammation of a bodily part.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligomerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “apply” as used herein refers to placing in contact with or to lay or spread on.

As used herein, the phrase “arginine compound” is used to refer to arginine, its salt, conjugate, or analog thereof.

The term “arginine” is used herein to describe a molecule or compound that comprises one amino group, one guanidino group, and one carboxylic group. Arginine is a solid and a known irritant of the skin and the eyes. At physiological pH, the carboxylic acid is deprotonated (—COO⁻), the amino group is protonated (—NH₃⁺), and the guanidino group is also protonated to give the guanidinium form (—C—(NH₂)²⁺), making arginine a positively charged, aliphatic amino acid.

Arginine has the molecular formula C₆H₁₅N₄O₂ and may be generally depicted as shown in Formula I:

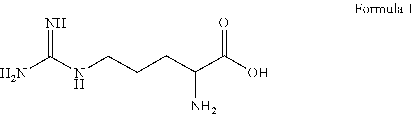 [SMILES: N=C(N)NCCCC(N)C(=O)O]

According to some embodiments, the arginine compound may be in one or more isomeric forms as represented by Formulas II and III below.

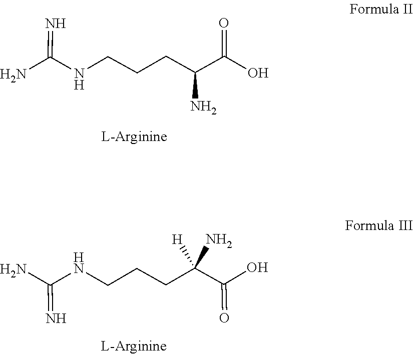 [SMILES: N=C(N)NCCC[C@H](N)C(=O)O ; N=C(N)NCCC[C@@H](N)C(=O)O]

L-arginine, the enantiomer of D-arginine, is also known as L(+)-Arginine, (2S)-2-amino-5-(carbamimidamido)pentanoic acid, (2S)-2-amino-5-guanidinopentanoic acid, (S)-2-amino-5-guanidinopentanoic acid, and (S)-2-amino-5-guanidinovaleric acid. It is considered the physiologically active isomer of arginine. It plays a role in a number of essential biochemical processes. L-arginine is a conjugate base (meaning it contains one less H atom and one more negative charge than the acid that formed it) of L-arginium(1+) and a conjugate acid (meaning it contains one more H atom and one more positive charge than the base that formed it) of L-arginate.

D-arginine, the enantiomer of L-arginine, is also known as D-2-amino guanidinovaleric acid, (2R)-2-amino-5-guanidinopentanoate. It believed that D-arginine may have properties slightly different from L-arginine (discussed supra). D-arginine is slightly soluble in water and is considered a moderately acidic compound based on its pKa. D-arginine is a conjugate base of a D-argininium(1+) and a conjugate acid of a D-argininate.

According to some embodiments, the arginine compound may be in one or more conjugate forms as represented by formulas IV and V below.

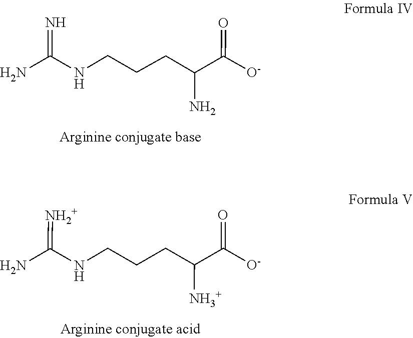 [SMILES: N=C(N)NCCCC(N)C(=O)[O-] ; NC(=[NH2+])NCCCC([NH3+])C(=O)[O-]]

According to some embodiments, the arginine compound may be in one or more analog forms as represented by Formula VI:

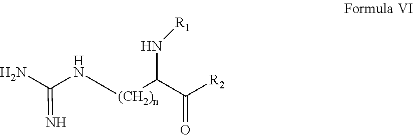

wherein
R₁ represents
a hydrogen atom, a hydroxyl group, an acyl or acyloxy radical, or an amino acid substituted or not on its free α-amino function, bound by a peptide bond;

R₂ represents

a hydroxyl group, an amine, alkylamine or alcoxy radical, a silyloxy group, or an aminoacid substituted or not on its free α-carboxylic function, bound by a peptide bond; and

n represent 3 or 4.

According to some embodiments, the arginine compound is obtained from a commercial source, including, but not limited to VladaChem, Tractus, Phion Ltd, MuseChem, MolPort, Sigma-Aldrich, LGC Standards, AN PharmaTech, ChemFaces, CAPOT, eNovation Chemicals, abcr GmbH, Tyger Scientific, Hairui Chemical, BLD Pharm, Biosynth, Aurum Pharmatech LLC, ApexBio Technology, ChemShuttle, Oakwood Products, DC Chemicals, AA BLOCKS, Yuhao Chemical, Norris Pharm, Tocris Bioscience, R&D Chemicals, Assembly Blocks Pvt. Ltd, Parchem, Angene Chemical, MedChemexpress MCE, Achemo Scientific Limited, Apexmol, Key Organics/BIONET, and the like. According to some embodiments, the arginine compound is synthesized.

The term “botanical raw material” as used herein refers to a fresh or processed (e.g., cleaned, frozen, dried, sliced or liquefied) part of a single species of plant or a fresh or processed alga or macroscopic fungus. The term “botanical ingredient” as used herein refers to a component that originates from a botanical raw material. The term “botanical product” refers to a finished labeled product that contains vegetable matter, which may include plant materials, algae, macroscopic fungi or a combination thereof. Depending in part on its intended use, a botanical product may be a food, drug or cosmetic. The term “botanical extract” as used herein refers to a product prepared by separating, by chemical or physical process, medicinally active portions of a plant from the inactive or inert components. The botanical extracts prepared according to some embodiments of the described invention may be obtained by means of a solvent, optionally under pressure and/or heat.

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “chemoattractant” as used herein refers to a substance which attracts motile cells of a particular type.

The term “chemokine” as used herein refers to a family of chemoattractant cytokines that play a pivotal role in leukocyte migration. They have been classified into four main subfamilies: CXC, CC, CX3C, and XC, all of them exerting their biological effects by interacting with G-protein-coupled receptors known as chemokine receptors. Chemokines work through concentration gradients mediating cell migration toward areas of high chemokine concentrations. In addition to their role in immune system communication, chemokines are also expressed by other cell types. For example, in the CNS, neurons constitutively express CX3CL1 (also known as fractalkine) to control microglia activation through the CX3CL1/CX3CR1 axis. Chemokines expression is induced after stroke by the action of proinflammatory cytokines, such as IL-1, TNFα, and IL-6 on resident and infiltrated cells of the ischemic tissue. Two main chemokine networks are thought to be the recruiters of neutrophils and monocytes into the ischemic tissue: the CXCL8 (IL-8)/CXCR2 and the CCL2/CCR2 axis. In addition, the CX3CL1/CX3CR1 axis and SDF-1 (CXCL12) have also an important role in stroke outcome and in neurorepair. Chemokine receptor CXCR4 is the receptor for stromal cell-derived factor-1, SDF-1.

The term “conjugate acid-base pair” as used herein refers to a proton donor and its corresponding deprotonated species, e.g., benzoic acid (donor) and benzoate (acceptor). The term “conjugate acid” as used herein refers to the acid member of a pair of compounds that differ from each other by gain or loss of a proton. A conjugate acid can release or donate a proton. The term “conjugate base” as used herein refers to is the species that remains after the acid has donated its proton; a conjugate base can accept a proton.

The term “combination” as used herein refers to an assemblage of separate parts or qualities. The term “combining” as used herein refers to putting or adding together.

The term “complementary” as used herein refers to combining in such a way as to enhance or emphasize the qualities of each other or another.

The term “complex” as used herein refers to a molecular entity formed by a loose association involving two or more component molecular entities. The bonding between the components is normally weaker than in a covalent bond. The strength of the complex is derived from the delocalization and sharing of charges. A coordination complex (meaning a compound or ion with a central usually metallic atom or ion combined by coordinate bonds with a definite number of surrounding ions, groups, or molecules), also called a coordination compound, may or may not be covalent.

The term “component” as used herein refers to a constituent part, element or ingredient.

The term “composition” as used herein refers to a mixture of ingredients.

The term “contact” and its various grammatical forms as used herein refers to a state or condition of touching or of immediate or local proximity.

The term “controlled release” is intended to refer to any active-containing formulation in which the manner and profile of release of the active from the formulation are regulated. This refers to immediate as well as non-immediate release formulations, with non-immediate release formulations including, but not limited to, sustained release and delayed release formulations. The term “delayed release” is used herein in its conventional sense to refer to a formulation in which there is a time delay between administration of the formulation and the release of the active therefrom. “Delayed release” may or may not involve gradual release of the active over an extended period of time, and thus may or may not be “sustained release.”

The term “cosmetic” as used herein refers to articles (excluding soap) intended to be rubbed, poured, sprinkled, or sprayed on, introduced into, or otherwise applied to the human body or any part thereof for cleansing, beautifying, promoting attractiveness, or altering the appearance, and articles intended for use as a component of any such articles.

The term “cosmetic composition” as used herein refers to a composition that is intended to be rubbed, poured, sprinkled, or sprayed on, introduced into, or otherwise applied to a subject or any part thereof for cleansing, beautifying, promoting attractiveness, or altering the appearance, or an article intended for use as a component of any such article, except that such term does not include soap.

The term “cosmetic effect” as used herein refers to a consequence of applying a cosmetic to the skin with the intention of improving its appearance or of beautifying it.

The term “cosmetically acceptable carrier” as used herein refers to a substantially non-toxic carrier, useable for administration of cosmetics, with which active compounds will remain stable and bioavailable. The carrier must be of sufficiently high purity and of sufficiently low toxicity to render it suitable for administration to the mammal being treated. It further should maintain the stability and bioavailability of an active agent. The cosmetically acceptable carrier is selected with the planned manner of administration in mind, to provide for the desired bulk, consistency, etc., when combined with an active agent and other components of a given composition.

The term “cytokine” as used herein refers to small soluble protein substances secreted by cells, which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Cytokines can act both locally and distantly from a site of release. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of other cytokines. Nonlimiting examples of cytokines include e.g., IL-1α, IL-β, IL-2, IL-3, IL-4, IL-5, IL-6, IL-7, IL-8, IL-9, IL-10, IL-11, IL-12/IL-23 P40, IL-13, IL-15, IL-17, IL-18, IL-21, IL-23, TGF-β, IFN-γ, GM-CSF, Gro-α, MCP-1 and TNF-α.

The term “dentate line” as used herein refers to an anatomic landmark that divides the upper two-thirds from the lower third of the anal canal.

The term “derivative” as used herein means a compound that may be produced from another compound of similar structure in one or more steps. A “derivative” or “derivatives” of a compound retains at least a degree of the desired function of the compound. Accordingly, an alternate term for “derivative” may be “functional derivative.” Derivatives can include chemical modifications of the compound, such as akylation, acylation, carbamylation, iodination or any modification that derivatizes the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives.

The term “dyspareunia” as used herein refers to pain during sexual intercourse.

The terms “emollient” or “moisturizer” as used herein are used interchangeably to refer to complex mixtures of chemical agents specially designed to make the external layers of the skin (epidermis) softer and more pliable. An emollient increases the skin's hydration (water content) by reducing evaporation.

The term “extracellular matrix” or “ECM” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

The terms “formulation” and “composition” are used interchangeably herein to refer to a product of the described invention that comprises all active and inert ingredients.

The term “finished product” as used herein refers to a cosmetic composition that has undergone all stages of production, including packaging in its final container.

The terms “formulation” and “composition” are used interchangeably herein to refer to a product of the described invention that comprises all active and inert ingredients.

The term “heal” and its various grammatical forms as used herein refers to making sound, well, or healthy again; to restore to a healthy condition.

The term “hydrophilic” as used herein refers to a material or substance having an affinity for polar substances, such as water. The term “hydrophobic” as used herein refers to a material or substance having an affinity for nonpolar or neutral substances.

The term “improve” and its various grammatical forms as used herein refers to bringing into a more desirable or excellent condition.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation includes the influx of lymphocytes and macrophages and fibroblast growth, which may result in tissue scarring at sites of prolonged or repeated inflammatory activity.

The term “immune system” as used herein refers to a complex network of cells, tissues, organs, and the substances they make that helps the body fight infections and other diseases. The immune system includes white blood cells and organs and tissues of the lymph system, such as the thymus, spleen, tonsils, lymph nodes, lymph vessels, and bone marrow. Responses in the immune system may generally be divided into two arms, referred to as “innate immunity” and “adaptive immunity”. The innate arm of the immune system is a nonspecific fast response to pathogens that is predominantly responsible for an initial inflammatory response via a number of soluble factors, including the complement system and the chemokine/cytokine system; and a number of specialized cell types, including mast cells, macrophages, dendritic cells (DCs), and natural killer cells (NKs). The adaptive immune arm involves a specific, delayed and longer-lasting response by various types of cells that create long-term immunological memory against a specific antigen. It can be further subdivided into cellular and humoral branches, the former largely mediated by T cells and the latter by B cells.

The terms “immunomodulatory”, “immune modulator”, “immunomodulatory,” and “immune modulatory” are used interchangeably herein to refer to a substance, agent, or cell that is capable of augmenting or diminishing immune responses directly or indirectly, e.g., by expressing chemokines, cytokines and other mediators of immune responses.

As used herein, the term “immunostimulatory amount” refers to an amount of an immunogenic composition that stimulates an immune response by a measurable amount, for example, as measured by ELISPOT assay (cellular immune response), ICS (intracellular cytokine staining assay) and major histocompatibility complex (MHC) tetramer assay

As used herein the term “immunosuppressive amount” refers to an amount of an immunosuppressive composition that suppresses an immune response, for example, as measured by ELISPOT assay (cellular immune response), ICS (intracellular cytokine staining assay) and major histocompatibility complex (MHC) tetramer assay.

As used herein, the term “inflammasome” refers to a pro-inflammatory protein complex that is formed after stimulation of the intracellular NOD-like receptors. Production of an active caspase in the complex processes cytokine proteins into active cytokines.

The term “injury” as used herein refers to the damage or wound of trauma.

The term “insult” as used herein refers to an injury, attack or trauma.

The term “interleukin (IL)” as used herein refers to a cytokine from a class of homologously related proteins that were first observed to be secreted by, and acting on, leukocytes. It has since been found that interleukins are produced by a wide variety of body cells. Interleukins regulate cell growth, differentiation, and motility, and stimulates immune responses, such as inflammation. Examples of interleukins include, interleukin-1 (IL-1), interleukin-1β (IL-1β), interleukin-6 (IL-6), interleukin-8 (IL-8), interleukin-12 (IL-12), and interleukin-17 (IL-17). Interleukin 1-α (IL-1A) and Interleukin 1β (IL-1B) are equally potent inflammatory cytokines that activate the inflammatory process [DiPaolo, N C and Shayakhmetov, D M, Nat. Immunol. (2016) 17 (8): 906-13]. Interleukin 6 (IL-6), which promptly and transiently produced in response to infections and tissue injuries, contributes to host defense through the stimulation of acute phase responses, hematopoiesis, and immune reactions. Although its expression is strictly controlled by transcriptional and posttranscriptional mechanisms, dysregulated continual synthesis of IL-6 plays a pathological effect on chronic inflammation and autoimmunity [Tanaka, T. et al. Cold Spring Harb. Perspect. Biol. (2014) 6 (10): a016295]. Interleukin 8 (IL-8) is a proinflammatory cytokine with proangiogenic, proliferative, and promotility activities. [Kondo, S. et al. J. Invest. Dermatol. (1993) 101 (5): 690-4].

The term “isolate” as used herein refers to an element or compound separated in substantially pure form from substances with which it may be associated in living systems or during synthesis. As used herein, the term “substantially pure” refers to a purity of at least 75%, at least 80%, at least 85%, at least 90%, at least 95% or at least 99% as determined by an analytical protocol.

The term “keratinocyte derived chemokine (KC/CXCL1) is one of the major attractants of neutrophils in the mouse and is a functional murine homolog of human CXCL1/Gro-α and CXCL8/IL-8. It binds to the chemokine receptor CXCR2 present on neutrophils. [See Chintakuntlawar, A V & Chodosh, J. J. Interferon Cytokine Res. (2009) 29 (10): 657-666].

The term “lymphocyte” refers to a small white blood cell (leukocyte) formed in lymphatic tissue throughout the body and in normal adults making up about 22-28% of the total number of leukocytes in the circulating blood, which plays a large role in defending the body against disease. Individual lymphocytes are specialized in that they are committed to respond to a limited set of structurally related antigens through recombination of their genetic material. This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from the epitopes that it can recognize. Lymphocytes differ from each other not only in the specificity of their receptors, but also in their functions (Id.). Two broad classes of lymphocytes are recognized. The B-lymphocytes (B-cells) are precursors of antibody-secreting cells. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippincott-Raven Publishers, Philadelphia, (1999)).

The term “maintain” as used herein refers to keeping, holding, supporting, preserving, or continuing in a healthy condition.

The term “matrix metalloproteinases (MMPs)” as used herein refers to zinc-dependent endopeptidases capable of degrading extracellular matrix molecules. The dynamic equilibrium between matrix metalloproteinases and their inhibitors is a critical determinant of matrix remodeling (R. Visse and H. Nagase, “Matrix metalloproteinases and tissue inhibitors of metalloproteinases: structure, function, and biochemistry,” Circulation Research, vol. 92, no. 8, pp. 827-839, 2003). Because these enzymes need zinc or calcium atoms to work properly, they are called metalloproteinases. Matrix metalloproteinases are involved in wound healing, angiogenesis, and tumor cell metastasis. For example, MMP-12 is a key regulator of macrophage infiltration and inflammation, contributing to retinal vascular dysfunction and pathological angiogenesis.

The term “macrophage inflammatory protein-1 alpha (MI-la)” as used herein refers to a chemotactic chemokine secreted by macrophages. It performs various biological functions, such as recruiting inflammatory cells, wound healing, inhibition of stem cells, and maintaining an effector immune response.

The term “microbiome” as used herein refers to a characteristic microbial community occupying a reasonable well-defined habitat which has distinct physio-chemical properties, which forms a dynamic and interactive micro-ecosystem prone to change in time and scale, and is integrated in macro-ecosystems including eukaryotic hosts. [Berg, G. et al. Microbiome (2020) 8: 103].

The term “microbiota” as used herein comprises all living members forming the microbiome.

The term “modulate” as used herein means to regulate, alter, adapt, or adjust to a certain measure or proportion.

The term “monocyte chemoattractant protein 1 (MCCP-1/CCL2)” as used herein refers to a key chemokine that regulates migration and infiltration of monocytes/macrophages.

The term “MTT assay” as used herein refers to a colorimetric reaction that can easily be measured from cell monolayers that have been plated in 35 mm dishes or multiwell plates. It has been widely used to assess cell viability and relies on the enzymatic reduction of 3-[4,5-dimethylthiazole-2-yl]-2,5-diphenyltetrazolium bromide (MTT) to MTT-formazan as by catalyzed by mitochondrial succinate dehydrogenase. Hence, the MTT assay is dependent on mitochondrial respiration and indirectly serves to assess the cellular energy capacity of a cell.

The term “neutrophilic granulocyte” or polymorphonuclear neutrophils (PMNs) as used herein refers to the most abundant white blood cell in humans and mice. They are characterized by the multi-lobed shape of their nucleus. Neutrophils are the first white blood cells recruited to sites of acute inflammation, in response to chemotactic cues such as CXCL8 (interleukin-8, IL-8) produced by stressed tissue cells and tissue-resident immune cells such as macrophages. Neutrophils display an array of biological functions important for both innate and adaptive immune responses. Neutrophils can produce many cytokines and chemokines upon stimulation, and in this way, they can interact with endothelial cells, dendritic cells, macrophages, natural killer cells, T lymphocytes, and B lymphocytes. Through all these interactions, neutrophils can either activate or downregulate both innate and adaptive immunity. [See Rosales, C. et al. J. Immunol. Res. (2017): 9748345].

The term “normal healthy control subject” as used herein refers to a subject having no symptoms or other clinical evidence of a vaginovulval or genitourinary trauma, insult or injury.

The term “nuclear factor-κB (NF-κB)” as used herein refers to a family of inducible transcription factors which regulate cytokine and chemokine signaling. The family switches on multiple inflammatory genes, including cytokines, chemokines, proteases, and inhibitors of apoptosis, resulting in amplification of the inflammatory response [Barnes, P J, (2016) Pharmacol. Rev. 68: 788-815]. The molecular pathways involved in NF-κB activation include several kinases. The classic (canonical) pathway for inflammatory stimuli and infections to activate NF-κB signaling involve the IKK (inhibitor of KB kinase) complex, which is composed of two catalytic subunits, IKK-α and IKK-β, and a regulatory subunit IKK-γ (or NFκB essential modulator [Id., citing Hayden, M S and Ghosh, S (2012) Genes Dev. 26: 203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to κB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β [Id., citing Sun, S C. (2012) Immunol. Rev. 246: 125-140]. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-89].

As used herein, the terms “occlude,” “occluded,” “occlusive” and the like refer to a transdermal formulation that is applied to the skin with the use of a supporting or otherwise associated structure. For example, a topical formulation may be applied to the skin of a subject with the aid of a structure, such as a backing member, bandage or cover. A matrix patch is an example of an occluded device. Conversely, the terms “unoccluded” and “non-occluded,” which may be used interchangeably, refer to a transdermal formulation that is applied to the skin without the use of a support, backing member, cover or otherwise associated structure. For example, the transdermal formulation is applied to the skin in a free form, which is sufficient to effect transdermal delivery of the active agent without the use of structures, such as a backing member, etc. A gel formulation is an example of a non-occluded composition; other non-occluded compositions include ointments, lotions, pastes, mousses, aerosols and creams.

The term “penetration” and its various grammatical forms as used herein refer to delivery of a substance through the skin.

The term “penetration enhancer” as used herein refers to an agent known to accelerate the delivery of a substance through the skin.

The term “percutaneous absorption” refers to the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug, include the establishment of a concentration gradient, which provides a driving force for drug movement across the skin, the release of drug from the vehicle into the skin-partition coefficient and drug diffusion across the layers of the skin-diffusion coefficient. The relationship of these factors to one another is summarized by the following equation:

J=Cᵥₑₕ×Kₘ·D/x [Formula VII]

where:
J=rate of absorption
Cᵥₑₕ=concentration of drug in vehicle
Kₘ=partition coefficient
D=diffusion coefficient

There are many factors which affect the rate of percutaneous absorption of a substance. Primarily they are as follows: (i) Concentration. The more concentrated the substance, the greater the absorption rate; (ii) Size of skin surface area to which the drug is applied. The wider the contact area of the skin to which the substance is applied, the greater the absorption rate; (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body; (iv) Hydration. Hydration (meaning increasing the water content of the skin) causes the stratum corneum to swell which increases permeability; (v) Skin temperature. Increased skin temperature increases permeability; and (vi) Composition. The composition of the compound and of the vehicle also determines the absorbency of a substance. Most substances applied topically are incorporated into bases or vehicles. The vehicle chosen for a topical application will greatly influence absorption, and may itself have a beneficial effect on the skin. Factors that determine the choice of vehicle and the transfer rate across the skin are the substance's partition coefficient, molecular weight and water solubility. The protein portion of the stratum corneum is most permeable to water soluble substances and the liquid portion of the stratum corneum is most permeable to lipid soluble substances. It follows that substances having both liquid and aqueous solubility can traverse the stratum corneum more readily. See Dermal Exposure Assessment: Principles and Applications, EPA/600/8-91/011b, January 1992, Interim Report—Exposure Assessment Group, Office of Health and Environmental Assessment, U.S. Environmental Protection Agency, Washington, D.C. 20460.

The term “petechiae” as used herein refers to minute hemorrhagic spots, of pinpoint to pinhead size, in the skin.

The terms “pH adjusting agent” as used herein refers to a substance used to achieve desired pH control in a formulation. Exemplary pH modifiers include acids (e.g., acetic acid, adipic acid, carbonic acid, citric acid, fumaric acid, lactic acid, phosphoric acid, sorbic acid, succinic acid, tartaric acid, basic pH modifiers (e.g., magnesium oxide, tribasic potassium phosphate, Eudragit® E), and pharmaceutically acceptable salts thereof.

The term “perianal” as used herein refers to being situated in or affecting the area around the anal orifice.

The term “plasminogen activator inhibitor-1 (PAI-1) as used herein refers to a member of the superfamily of serine-protease inhibitors (or serpins), and the principal inhibitor of both the tissue-type and the urinary-type plasminogen activator, the two plasminogen activators able to activate plasminogen. Plasminogen activators convert plasminogen to plasmin.

The term “polymer” as used herein refers to any of various chemical compounds made of smaller, identical molecules (called monomers) linked together. Polymers generally have high molecular weights. The process by which molecules are linked together to form polymers is called “polymerization.”

The term “preservative” as used herein refers to a substance that is added to a product to prevent decomposition by microbial growth or by undesirable chemical changes.

The term “promote” as used herein in a medical context refers to a process of enabling increased control over vaginovulval and/or perianal tissue health and its determinants.

The terms “psychoactive” and “psychotropic” as used herein refer to producing an effect (such as changes in perception or behavior) on the mind or on mental processes.

The term “purification” and its various grammatical forms as used herein refers to a process of isolating or freeing from foreign, extraneous, or objectionable elements.

The term “pyrin” as used herein refers to one of several protein interaction domains structurally related to but distinct from CARD, TIR, DD and DED domains.

The term “pyroptosis” as used herein refers to a form of programmed cell death that is associated with abundant pro-inflammatory cytokines such as IL-1β and IL-18 produced through inflammasome activation.

The term “radiation therapy” as used herein refers to the use of high-energy x-rays to kill cancer cells. Radiation may be used alone or with other treatments to effectively treat gynecologic cancers. Radiation therapy may include both external beam radiation therapy using a linear accelerator, and internal radiation called brachytherapy. Internal radiation is given using a device called high dose rate (HDR) brachytherapy. These HDR treatments that are often referred to as “implants” involve placing an apparatus inside the body very close to the cancer, and loading radiation into that apparatus and delivering a high dose of radiation over a short period of time. Internal radiation is used when a dose of radiation needs to be delivered to a small area, by placing the radiation as close as possible to the cancer cells; this allows for a dose of radiation to be delivered to the cancer while the normal near-by organs get a very low dose.

The term “reactive oxygen species” or “ROS”, such as free radicals and peroxides, represent a class of molecules that are derived from the metabolism of oxygen and exist inherently in all aerobic organisms. Most reactive oxygen species come from endogenous sources as byproducts of normal and essential metabolic reactions, such as energy generation from mitochondria or the detoxification reactions involving the liver cytochrome P-450 enzyme system.

The term “redox imbalance” as used herein refers to an overproduction of reactive oxygen species or reactive nitrogen species that overwhelm the protective defense mechanism of cells. Consequences of this redox imbalance are lipid peroxidation, oxidation of proteins, DNA damage, and interference of reactive oxygen species with signal transduction pathways.

The term “reduce” and its various grammatical forms as used herein refers to a diminution, a decrease, an attenuation or abatement of a degree, intensity, extent, size, amount, density or number.

The term “rejuvenate” and its various grammatical forms as used herein refers to bringing back to a youthful state.

The term “repair” as used herein refers to mending or restoring to a sound or good state after injury or decay.

The term “restore” and its various grammatical forms as used herein refers to bringing back to a former or normal condition, to recover or renew.

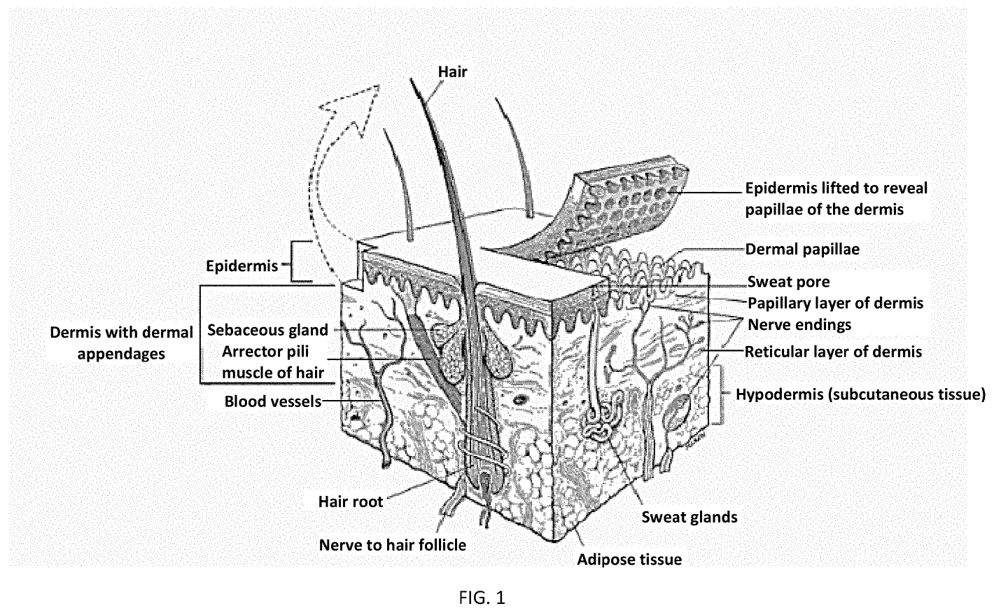

The term “skin” as used herein refers to the largest organ in the body consisting of several layers. It plays an important role in biologic homeostasis, and is comprised of the epidermis and the dermis. The epidermis, which is composed of several layers beginning with the stratum corneum, is the outermost layer of the skin, and the innermost skin layer is the deep dermis. The skin has multiple functions, including thermal regulation, metabolic function (vitamin D metabolism), and immune functions. FIG. 1 presents a diagram of skin anatomy.

In humans, the usual thickness of the skin is from 1-2 mm, although there is considerable variation in different parts of the body. The relative proportions of the epidermis and dermis also vary, and a thick skin is found in regions where there is a thickening of either or both layers. For example, on the interscapular (between the shoulder blades) region of the back, where the dermis is particularly thick, the skin may be more than 5 mm thick, whereas on the eyelids it may be less than 0.5 mm. Generally, the skin is thicker on the dorsal or extensor surfaces of the body than on the ventral or flexor surfaces; however, this is not the case for the hands and feet. The skin of the palms and soles is thicker than on any dorsal surface except the intrascapular region. The palms and soles have a characteristically thickened epidermis, in addition to a thick dermis

The entire skin surface is traversed by numerous fine furrows, which run in definite directions and cross each other to bound small rhomboid or rectangular fields. These furrows correspond to similar ones on the surface of the dermis so that, in section, the boundary line between epidermis and dermis appears wavy. On the thick skin of the palms and soles, the fields form long, narrow ridges separated by parallel coursing furrows, and in the fingertips these ridges are arranged in the complicated loops, whorls (verticil) and spirals that give the fingerprints characteristic for each individual. These ridges are more prominent in those regions where the epidermis is thickest.

Where there is an epidermal ridge externally there is a corresponding narrower projection, called a “rete peg,” on the dermal surface. Dermal papillae on either side of each rete peg project irregularly into the epidermis. In the palms and soles, and other sensitive parts of the skin, the dermal papillae are numerous, tall and often branched, and vary in height (from 0.05 mm to 0.2 mm). Where mechanical demands are slight and the epidermis is thinner, such as on the abdomen and face, the papillae are low and fewer in number.

The epidermis provides the body's buffer zone against the environment. It provides protection from trauma, excludes toxins and microbial organisms, and provides a semi-permeable membrane, keeping vital body fluids within the protective envelope. Traditionally, the epidermis has been divided into several layers, of which two represent the most significant ones physiologically. The basal-cell layer, or germinative layer, is of importance because it is the primary source of regenerative cells. In the process of wound healing, this is the area that undergoes mitosis in most instances. The upper epidermis, including stratum and granular layer, is the other area of formation of the normal epidermal-barrier function.

The stratum corneum is an avascular, multilayer structure that functions as a barrier to the environment and prevents transepidermal water loss. Recent studies have shown that enzymatic activity is involved in the formation of an acid mantle in the stratum corneum. Together, the acid mantle and stratum corneum make the skin less permeable to water and other polar compounds, and indirectly protect the skin from invasion by microorganisms. Normal surface skin pH is between 4 and 6.5 in healthy people; it varies according to area of skin on the body. This low pH forms an acid mantle that enhances the skin barrier function.

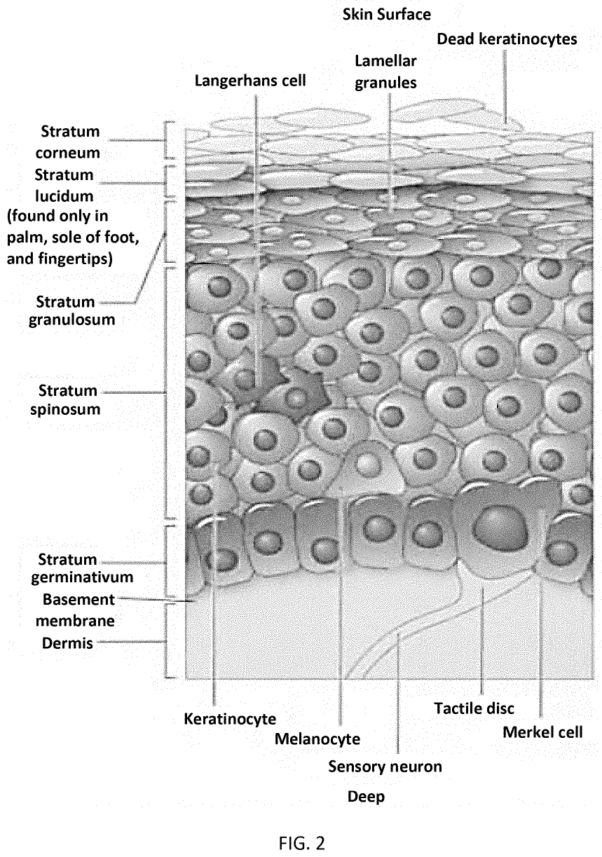

Other layers of the epidermis below the stratum corneum include the stratum lucidum, stratum granulosum, stratum germinativum, and stratum basale. See FIG. 2. Each contains living cells with specialized functions For example melanin, which is produced by melanocytes in the epidermis, is responsible for the color of the skin. Langerhans cells are involved in immune processing.

Dermal appendages, which include hair follicles, sebaceous and sweat glands, fingernails, and toenails, originate in the epidermis and protrude into the dermis hair follicles and sebaceous and sweat glands. They contribute epithelial cells for rapid re-epithelialization of wounds that do not penetrate through the dermis (termed partial-thickness wounds). The sebaceous glands are responsible for secretions that lubricate the skin, keeping it soft and flexible. They are most numerous in the face and sparse in the palm of the hands and soles of the feet. Sweat gland secretions control skin pH to prevent dermal infections. The sweat glands, dermal blood vessels, and small muscles in the skin (responsible for goose pimples) control temperature on the surface of the body. Nerve endings in the skin include receptors for pain, touch, heat, and cold. Loss of these nerve endings increases the risk for skin breakdown by decreasing the tolerance of the tissue to external forces.

The basement membrane both separates and connects the epidermis and dermis. When epidermal cells in the basement membrane divide, one cell remains, and the other migrates through the granular layer to the surface stratum corneum. At the surface, the cell dies and forms keratin. Dry keratin on the surface is called scale. Hyperkeratosis (thickened layers of keratin) is found often on the heels and indicates loss of sebaceous gland and sweat gland functions if the patient is diabetic. The basement membrane atrophies with aging; separation between the basement membrane and dermis is one cause for skin tears in the elderly.

The dermis, or the true skin, is a vascular structure that supports and nourishes the epidermis. In addition, there are sensory nerve endings in the dermis that transmit signals regarding pain, pressure, heat, and cold. The dermis is divided into two layers: the superficial dermis and the deep dermis.

The superficial dermis consists of extracellular matrix (collagen, elastin, and ground substances) and contains blood vessels, lymphatics, epithelial cells, connective tissue, muscle, fat, and nerve tissue. The vascular supply of the dermis is responsible for nourishing the epidermis and regulating body temperature. Fibroblasts are responsible for producing the collagen and elastin components of the skin that give it turgor. Fibronectin and hyaluronic acid are secreted by the fibroblasts. The structural integrity of the dermis plays a role in the normal function and youthful appearance of the skin.

The deep dermis is located over the subcutaneous fat; it contains larger networks of blood vessels and collagen fibers to provide tensile strength. It also consists of fibroelastic connective tissue, which is yellow and composed mainly of collagen. Fibroblasts are also present in this tissue layer. The well-vascularized dermis withstands pressure for longer periods of time than subcutaneous tissue or muscle. The collagen in the skin gives the skin its toughness. Dermal wounds, e.g., cracks or pustules, involve the epidermis, basal membrane, and dermis. Typically, dermal injuries heal rapidly.

Substances are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations.

The terms “soluble” and “solubility” as used herein refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent.

According to the European Pharmacopoeia, the solubility of a compound in water in the range of 15° C. to 25° C. is defined as follows:

| | Solvent in mL per gram compound |
|---|---|
| Very readily soluble | <1 |
| Readily soluble | from 1 to 10 |
| Soluble | from >10 to 30 |
| Hardly soluble | from >30 to 100 |
| Poorly soluble | from >100 to 1,000 |
| Very poorly soluble | from >1,000 to 10,000 |
| Water-insoluble | >10,000 |

The term “solubilizing agents” as used herein refers to those substances that enable solutes to dissolve.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “soothing” as used herein refers to reducing pain or discomfort.

The term “stable” and its various grammatical forms as used herein refers to the capability of a particular formulation to remain within its physical, chemical, microbiological, therapeutic and toxicological specifications.

The term “stabilizer” as used herein refers to a chemical which tends to inhibit the reaction between two or more other chemicals.

The term “subject in need thereof” as used herein refers to a female subject susceptible to or experiencing vaginovulval or genitourinary symptoms of trauma, insult or injury.

The term “suspension” as used herein refers to a dispersion (mixture) in which a finely-divided species is combined with another species, with the former being so finely divided and mixed that it doesn't rapidly settle out. In everyday life, the most common suspensions are those of solids in liquid.

The term “susceptible” as used herein refers to a member of a population at risk. The term “susceptible population” as used herein refers to a subpopulation in the general population likely to experience a disease.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period.

The term “synergistic effect” as used herein refers to an interaction between two or more agents that causes the total effect of the agents to be greater than the sum of the individual effects of each drug

The term “symptom” as used herein refers to a sign or an indication of disorder or disease, especially when experienced by an individual as a change from normal function, sensation, or appearance.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The terms “therapeutic amount”, “cosmetic amount” or an “amount effective” of one or more of the active agents as used herein refer to an amount that is sufficient to provide the intended benefit of treatment.

The term “tissue” as used herein refers to a collection of cells that act together to perform a specific function. There are four basic tissues in the body: 1) epithelium; (2) the connective tissues, including blood, bone and cartilage; (3) muscle tissue and 4) nerve tissue. In healthy tissues, the cells stay in place where they are, adhering to each other in structures that characterize the tissue and assist in its function.

### Toll-Like Receptors

Toll-like receptors (TLRs) are sensors for microbes present in extracellular spaces. Some are cell surface receptors (e.g., TLR-1, TLR-2, TLR-5, TLR-6), but others (e.g., TLR3, TLR-7, TLR-8, TLR-9) are located intracellularly in the membrane of endosomes, where they detect pathogens or their components that have been taken into cells by phagocytosis, receptor-mediated endocytosis or micropinocytosis. Janeway's Immunology, 9th Ed. 2017, Garland Science, New York, at 88.

TLR-4, is expressed by several types of immune system cells, including dendritic cells and macrophages, recognizes the LPS of gram negative bacteria by a mechanism that is partly direct and partly indirect. Systemic injection of LPS causes a collapse of the circulatory and respiratory system (shock), due to an overwhelming secretion of cytokines, particularly TNF-α, causing systemic vascular permeability. To recognize LPS, the ectodomain of TLR-4 uses an accessory protein, MD-2, which initially binds to TLR-4 within the cell and is necessary both for the correct trafficking of TLR-4 to the cell surface and for the recognition of LPS. TLR-4 activation involves two other accessory proteins, LPS-binding protein, present in the blood and in extracellular fluid in tissues, and CD14, which is present on the surface of macrophages, neutrophils and dendritic cells. On its own, CD14 can act as a phagocytic receptor, but on macrophages and dendritic cells it also acts as an accessory protein for TLR-4. Janeway's Immunology, 9th Ed. 2017, Garland Science, New York, at 92.

Mammalian TLRs recognize molecules characteristic of bacteria, fungi and viruses, including the lipoteichoic acids of Gram-positive bacterial cell walls, and the lipopolysaccharide (LPS) of the outer membrane of Gram negative bacteria. Although TLRs have limited specificity compared with the antigen receptors of the adaptive immune system, they can recognize elements of most pathogenic microbes and are expressed by many types of cells, including macrophages, dendritic cells, B cells, stromal cells, and certain epithelial cells. Janeway's Immunology, 9th Ed. 2017, Garland Science, New York, at 88.

Signaling by mammalian TLRs in various cell types induces a diverse range of intracellular responses by activating several different signaling pathways that each activate different transcription factors, which, together result in the production of inflammatory cytokines, chemotactic factors, antimicrobial peptides, and the antiviral cytokines interferon α and β. Janeway's Immunology, 9th Ed. 2017, Garland Science, New York, at 92. The outcome of TLR activation can vary depending on the cell type in which it occurs. Janeway's Immunology, 9th Ed. 2017, Garland Science, New York, at 95.

Signaling by mammalian TLRs is activated when binding of a ligand induces formation of a dimer, or induces conformational changes in a preformed TLR dimer. All mammalian TLR proteins have in their cytoplasmic tail a Toll-IL-1 receptor (TIR) domain, which interacts with other T1R-type domains, usually in other signaling molecules, and is also found in the cytoplasmic tail of the receptor for the cytokine interleukin-1-β. Id. At 88 Dimerization brings the cytoplasmic T1R domains together, allowing them to interact with the T1R domains of cytoplasmic adaptor molecules that initiate intracellular signaling. There are four adaptors used by mammalian TLRs: MyD88, MAL (also known as TIRAP), TRIF, and TRAM. The T1R domains of the different TLRs interact with different combinations of these adaptors [Janeway's Immunology, 9th Ed. 2017, Garland Science, New York, at 92-93].

For example, TLR-3 interacts only with TRIF. Signaling by the TLR-2 heterodimers (TLR-2/1 and TLR2/6) requires MyD88/MAL. TLR-4 signaling uses both MyD8/MAL and TRIF/TRAM, which is used during endosomal signaling by TLR-4. The choice of adaptor influences which of the several downstream signals will be activated by the TLR [Janeway's Immunology, 9th Ed. 2017, Garland Science, New York, at 94].

Signaling by most TLRs activates the transcription factor NFκB, several members of the interferon regulatory factor (IRF) transcription factor family through a second pathway, and members of the activator protein 1 (AP-1) family, such as c-Jun, through another signaling pathway involving mitogen-activated protein kinases (MAPKs). NFκB and AP-1 act primarily to induce the expression of proinflammatory cytokines and chemotactic factors[Janeway's Immunology, 9th Ed. 2017, Garland Science, New York, at 94].

The Signaling Pathway Triggered by TLRs that Use MyD88

TLR-7, TLR-8 and TLR-9 signal uniquely through MyD88. MyD88 has a T1R domain at its carboxy terminus that associates with the T1R domains in the TLR cytoplasmic tails. At its amino terminus, it has a death domain that associates with a similar death domain present in other intracellular signaling proteins. Both domains are required for signaling. The MyD88 death domain recruits and activates two serine-threonine protein kinases—IRAK4 (IL-1 receptor associated kinase 4) and IRAK1—via their death domains. This IRAK complex recruits enzymes that produce a signaling scaffold, and uses this scaffold to recruit other molecules that are then phosphorylated by the IRAKs [Janeway's Immunology, 9th Ed. 2017, Garland Science, New York, at 94]. To form a signaling scaffold, the IRAK complex recruits the enzyme tumor necrosis factor receptor-associated factor 6 (TRAF6), which is an E3 ubiquitin ligase that acts in cooperation with UBC13, an E2 ubiquitin ligase, and its cofactor UvelA (together called TRIKA1). The combined activity of TRAF-6 and UBC13 is to ligate one ubiquitin molecule to another protein and thereby generate protein polymers. A polyubiquitin polymer, which can be initiated on other proteins, including TRAF-6 itself, can be extended to produce polyubiquitin chains that act as a scaffold that binds to other signaling molecules. Next the scaffold recruits a signaling complex consisting of the polyubiquitin-binding adaptor proteins TAB1, TAB2, and the serine-threonine kinase TAK1. TAK1 is phosphorylated by the IRAK complex, and activated TAK1 propagates signaling by activating certain MAPKs, such as c-Jun terminal kinase (JNK) and mAPK14 (p38 MAPK). These then activate AP-1 family transcription factors that transcribe cytokine genes.

TAK1 also phosphorylates and activates IκB kinase (IKK) complex, which is composed of three proteins: IKKα, IKKβ, and IKKγ (also known as NEMO, for NFκB essential modifier). NEMO binds to polyubiquitin chains, which brings the IKK complex into proximity with TAK1. TAK1 phosphorylates and activates IKKβ, which phosphorylates IκB (inhibitor of κB), a cytoplasmic protein that constitutively binds to transcription factor NFκB. NFκB contains two subunits, p50 and p65. The binding of IκB traps the NFκB proteins in the cytoplasm. Phosphorylation by IKK induces the degradation of IκB, which releases NFκB into the nucleus, where it can drive the transcription of genes for pro-inflammatory cytokines, such as TNF-α, IL-10, and IL-6 [Janeway's Immunology, 9th Ed. 2017, Garland Science, New York, at 94-95].

### TLR-3, TLR-7. TLR-8, and TLR-9, the Nucleic Acid-Sensing TLRs-Activate Members of the IRF Family.

IRF proteins reside in the cytoplasm and are inactive until they become phosphorylated on serine and threonine residues in their carboxy termini. They then move to the nucleus as active transcription factors. There are 9 IRF family members, of which IRF3 and IRF7 are particularly important for TLR signaling and expression of antiviral type 1 interferons. For TLR-3, which is expressed by macrophages and conventional dendritic cells, the cytoplasmic T1R domain interacts with adaptor protein TRIF, which interacts with E3 ubiquitin ligase TRAF3, which, like TRAF6, generates a polyubiquitin scaffold. In TLR-3 signaling, this scaffold recruits a multiprotein complex containing the kinases IKKε and TBK1, which phosphorylate IRF3. TLR-4 also triggers this pathway by binding TRIF, but the IRF3 response induced by TLR-4 is relatively weak compared with that induced by TLR-3.

For TLR-7 and TLR-9 signaling in plasmacytoid dendritic cells, the MyD88 T1R domain recruits the IRAK1/IRAK4 complex, which can also physically associate with IRF7, which is highly expressed by plasmacytoid dendritic cells. This allows IRF7 to become phosphorylated by IRAK1, leading to induction of type 1 interferons. Not all IRF factors regulate type 1 interferon genes; IRF5, for example, plays a role in induction of pro-inflammatory cytokines.

### NOD-Like Receptors (NLRs)

Nucleotide-binding oligomerization domain (NOD)-like receptors (NLRs) are innate sensors that detect microbial products or cellular damage in the cytoplasm or activate signaling pathways, and are expressed in cells that are routinely exposed to bacteria, such as epithelial cells, macrophages and dendritic cells.

Some NLRs activate NFκB to initiate the same inflammatory responses as the TLRs, while others trigger a distinct pathway that induces cell death and the production of pro-inflammatory cytokines [Janeway's Immunology, 9th Ed. 2017, Garland Science, New York, at 96].

Subfamilies of NLRs can be distinguished based on the other protein domains they contain. For example the NOD subfamily has an amino-terminal caspase recruitment domain (CARD), which is structurally related to the T1R death domain in MyD88, and can dimerize with CARD domains on other proteins to induce signaling. NOD proteins recognize fragments of bacterial cell wall peptidoglycans, although it is not known if they do so through direct binding or through accessory proteins [Janeway's Immunology, 9th Ed. 2017, Garland Science, New York, at 96]. NOD1 senses γ-glutamyl diaminopimelic acid (iE-DAP), a breakdown product of peptidoglycans of Gram negative and some Gram positive bacteria, whereas NOD2 recognizes muramyl dipeptide (MDP), which is present in the peptidoglycans of most bacteria. Id. Other members of the NOD family, including NLRX1 and NLRC5, have been identified, but their function is less well understood [Janeway's Immunology, 9th Ed. 2017, Garland Science, New York, at 96-98].

When NOD1 or NOD2 recognizes its ligand, it recruits the CARD-containing serine-threonine kinase RIP2 (also known as RICK and RIPK2), which associates with the E3 ligases cIAP1, CIAP2, and XIAP, whose activity generates a polyubiquitin scaffold, which recruits TAK1 and IKK and results in activation of NFκB. NFκB then induces the expression of genes for inflammatory cytokines and for enzymes involved in the production of NO [Janeway's Immunology, 9th Ed. 2017, Garland Science, New York, at 97].

Macrophages and dendritic cells express both TLFs and NOD1 and NOD2, and are activated by both pathways. In epithelial cells, NOD1 may also function as a systemic activator of innate immunity. NOD2 is strongly expressed in the Paneth cells of the gut where it regulates the expression of potent anti-microbial peptides such as the α- and β-defensins [Janeway's Immunology, 9th Ed. 2017, Garland Science, New York, at 97].

Other members of the NOD family, including NLRX1 and NLRC5, have been identified, but their function is less well understood [Janeway's Immunology, 9th Ed. 2017, Garland Science, New York, at 96-98].

The NLRP family, another subfamily of NLR proteins, has a pyrin domain in place of the CARD domain at their amino termini. Humans have 14 NLR proteins containing pyrin domains, of which NLRP3 (also known as NAPL3 or cryopyrin) is the best characterized. NLRP3 resides in an inactive form in the cytoplasm, where its leucine rich repeat (LRR) domains are thought to bind the head-shock chaperone protein HSP90 and the co-chaperone SGT1. NRLP3 signaling is induced by reduced intracellular potassium, the generation of reactive oxygen species, or the disruption of lysosomes by particulate or crystalline matter. For example, death of nearby cells can release ATP into the extracellular space, which would active the purinergic receptor P2X7, which is a potassium channel, and allow potassium ion efflux. A model proposed for ROS-induced NLRP3 activation involves intermediate oxidation of sensor proteins collectively called thioredoxin (TRX). Normally TRX proteins are bound to thioredoxin-interacting protein (TXNIP). Oxidation of TRX by ROS causes dissociation of TXNIP from TRX. The free TXNIP may then displace HSP90 and SGT1 from NLRP3, again causing its activation. In both cases, NLRP3 activation involves aggregation of multiple monomers via their leucine-rich repeat (LRR) and NOD domains to induce signaling. Phagocytosis of particulate matter (e.g. the adjuvant alum), may lead to the rupture of lysosomes and release of the active protease cathepsin B, which can activate NLRP3 [Janeway's Immunology, 9th Ed. 2017, Garland Science, New York, at 98-99].

NLR signaling, as exemplified by NLRP3, leads to the generation of pro-inflammatory cytokines and to cell death through formation of an inflammasome, a multiprotein complex. Activation of the inflammasome proceeds in several stages: (1) Aggregation of NLRP molecules triggers autocleavage of procaspase I, which releases active caspase 1—Aggregation of LRR domains of several NLRP3 molecules, or other NLRP molecules by a specific trigger or recognition event, which induces the pyrin domains of NLRP3 to interact with pyrin domains of ASC (also called PYCARD), an adaptor protein composed of an amino terminal pyrin domain and a carboxyterminal CARD domain, which further drives the formation of a polymeric ASC filament, with the pyrin domains in the center and the CARD domains facing outward; the CARD domains then interact with CARD domains of the inactive protease pro-caspase 1, initiating its CARD-dependent polymerization into discrete caspase 1 filaments. Active caspase 1 then carries out ATP-dependent proteolytic processing of proinflammatory cytokines, particularly IL-1β and IL-18, into their active forms, and induces a form of cell death (pyroptosis) associated with inflammation because of the release of these pro-inflammatory cytokines upon cell rupture[Janeway's Immunology, 9th Ed. 2017, Garland Science, New York, at 99-100].

The term “topical” as used herein refers to administration of an inventive composition at, or immediately beneath, the point of application. The term “topical administration” and “topically applying” as used herein are used interchangeably to refer to delivering a composition onto one or more surfaces, including epithelial surfaces. For example, the composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect.

The term “treat” or “treating” includes abrogating, substantially inhibiting, slowing or reversing the progression of a disease, condition or disorder, substantially ameliorating clinical or esthetic symptoms of a condition, substantially preventing the appearance of clinical or esthetic symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in subjects that have previously had the disorder(s); and (e) limiting recurrence of symptoms in subjects that were previously asymptomatic for the disorder(s).

The term “trauma” as used herein refers to an injury or wound to living tissue caused by an extrinsic agent.

The term “tumor necrosis factor alpha” or TNFα as used herein refers to a cytokine made by white blood cells in response to an antigen or infection, which induce necrosis (death) of tumor cells and possesses a wide range of pro-inflammatory actions. Tumor necrosis factor also is a multifunctional cytokine with effects on lipid metabolism, coagulation, insulin resistance, and the function of endothelial cells lining blood vessels.

The term “vascular endothelial growth factor A” or “VEGFA” as used herein refers to a heparin-binding protein, which exists as a disulfide-linked homodimer. This growth factor induces proliferation and migration of vascular endothelial cells, and is essential for both physiological and pathological angiogenesis.

The term “viscosity”, as used herein refers to the property of a fluid that resists the force tending to cause the fluid to flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by the fluid. Dynamic viscosity is usually denoted in poise (P) or centipoise (cP), wherein 1 poise=1 g/cm², and 1 cP=0.01 P. Kinematic viscosity is the ratio of absolute or dynamic viscosity to density. Kinematic viscosity is usually denoted in Stokes (St) or Centistokes (cSt), wherein 1 St=10-4 m²/s, and 1 cSt=0.01 St.

The term “vagina” as used herein refers to the genital canal in the female, extending from the uterus to the vulva.

The term “Vaginal Health Index (VHI) score” refers to a tool that, by evaluating 5 parameters (vaginal elasticity, vaginal secretions, pH, epithelial mucous membrane, vaginal hydration), allows the determination of a final score defining the degree of atrophy in the genitourinary tract by assigning a single score to each parameter. The score can vary between five and 25, with a cut-off <15 index of atrophic vagina. [See Alvisi, S. et al. Medicina (2019) 55 (10): 615].

The Vaginal Maturation Index is a score that indicates the degree of tissue maturation, measuring the percentage of superficial, intermediate, and parabasal cells. [See Alvisi, S. et al. Medicina (2019) 55 (10): 615]. The maturation value (MV) is calculated with the following formula: MV=% surface cells+(0.5×% intermediate cells).

The term “vaginovulvar” means relating to the vagina and vulva.

The term “vitality” as used herein refers to features that distinguish living from not living that are essential or necessary to the existence, continuance or well-being of a full and healthy life.

The term “volume/volume percentage (v/v %)” refers to a measure of the concentration of a substance in a solution. It is expressed as the ratio of the volume of the solute to the total volume of the solution multiplied by 100.

The term “vulva” as used herein refers to the external genitalia of the female, composed of the mons pubis, the labia majora and minora, the clitoris, the vestibule of the vagina and its glands, and the opening of the urethra and of the vagina.

The Vulvar Health Index (VHI) is a score that can be used to evaluate the vulva including vulvar inflammation, musculature contraction, pain at speculum insertion, and epithelial integrity. The score can vary from zero to 24, with a cut-off >8 index of atrophic vulva. [See Alvisi, S. et al. Medicina (2019) 55 (10): 615].

The term “wound healing” or “wound repair” as used herein refers generally to the body's natural process of regenerating dermal or epidermal tissue. When an individual is wounded, a set of complex biochemical events takes place to repair the damage including, hemostasis, inflammation, proliferation, and remodeling.

The term “wound healing agent” as used herein refers to an agent that promotes an intricate process where the skin or other body tissue repairs itself after injury. In normal skin, the epidermis (surface layer) and dermis (deeper layer) form a protective barrier against the external environment. As such, the term “wound healing agent” refers to any substance that facilitates the wound healing process.

As used herein, a “wt %” or “weight percent” or “percent by weight” or “wt/wt %” of a component, unless specifically stated to the contrary, refers to the ratio of the weight of the component to the total weight of the composition in which the component is included, expressed as a percentage.

### Embodiments

According to one aspect, the described invention provides a cosmetic composition formulated for topical application comprising a water-based gel; a botanical ingredient, and a cosmetic composition stabilizing system as described in U.S. patent application Ser. No. 16/867,370, which is incorporated herein by reference.

### Water-Based Gel Component

### Background

Hyaluronic acid (HA) is a member of the large family of glycosaminoglycans (GAGs), which are the main components of the extracellular matrix. The HA molecule is composed of a repeating unit of D-glucuronic acid and N-acetyl-D-glucosamine bound with β-glycosidic linkages.

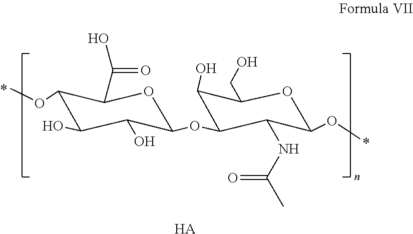

This simple molecular unit forms a long linear polymer, with molecular weight reaching 5×10⁶ kDa. Long hyaluronan polymers have the ability to bind large amounts of water.

In its native form of a very long polymer, hyaluronic acid (also known as hyaluronanor HA) is known as high molecular weight hyaluronic acid (HMWHA). In certain conditions, it can be decomposed into small fragments referred to as low molecular weight HA (“LMWHA”). [Litwiniuk, M. et al. Wounds (2016) 28 (3): 78-88, citing Aya, K L & Stern, R. Wound Repair Regen. (2014) 22(5): 579-93]. In somatic tissues, hyaluronidase-1 (Hyal-1) and hyaluronidase-2 (Hyal-2) are responsible for HA degradation. First, Hyal-2, a cell-membrane liked enzyme, degrades HA to fragments with molecular eight reaching 20 kDa. These HA molecules are subsequently endocytosed and delivered to lysosomes, where further digestion is performed by Hyal-1. [Id., citing Stern, R. & Jedrzejas, M. Chem. Rev. (2006) 106 (30: 818-39). In injured tissue, free radicals also can decompose HA polymers into smaller fragments. [Id., citing Longacre, M T et al. Ann. Surg. (1991) 213 (4): 292-96].

It has been well documented that HMWHA displays anti-inflammatory and immunosuppressive properties, whereas LMWHA degradation products of HA can induce inflammation. [Id., citing Prevo, R. et al. J. Biol. Chem. (2001) 276 (22): 19420-30]. Small hyaluronan fragments have been shown to increase the expression and protein production of several cytokines, such as MMP-12, plasminogen activator inhibitor-1 [Id., citing Horton, M R et al. Am. J. Physiol. Lung Cell Mol. Physiol. (2000) 279 (4): L707-15; Horton, M R, et a. J. Immunol. (1999) 162 (7): 4171-76], macrophage inflammatory protein-1a (MIP-1a), monocyte chemoattractant 1, keratinocyte chemoattractant, interleukin-8 (IL-8) and IL-12 by macrophages. [Id., citing Horton, M R, et al. J. Immunol. (1998) 160 (6): 3023-30; Hodge-DuFour, J. et al. J. Immunol. (1997) 159 (5): 2492-2500; McKee, C M et al. J. Clin. Invest. (1996) 98 (10): 1403-13]. While CD44 is the main receptor for hyaluronan, other receptors, such as TLRs also are involved in HA signaling. [Id., citing Teder, P. et al. Science (2002) 296 (5565): 155-58]. LMWHA is able to bind to TLR receptors and initiate the signaling cascade leading to the production of pro-inflammatory cytokines and chemokines in various types of cells in vitro. [Id., citing Litwiniuk, M. et al. Cent. Eur. J. Immunol. (2009) 34 (4): 247-51]. In immune cells from injured tissues, TLR2 and TLR4 activation by LMWHA has been shown to lead to initiation of MyD88-dependent NFκB signaling cascade and pro-inflammatory cytokine gene expression. [Id., citing Jiang, D. et al. Nat. Med. (2005) 11 (11): 1173-79; Suga, H. et al. J. Dermatol. Sci. (2014) 73 (2): 117-24]. Induction by LMWHA TLR-related myeloid differentiation primary response gene 88 (MyD88)/NFκB signaling also was confirmed in breast tumor cells. Small HA fragments were shown to stimulate CD44 association with TLR2, TLR4 and MyD88, leading to NF-κB-specific transcriptional activation and the expression of proinflammatory cytokines Il-1β and IL-8 in a human breast cell line. Taken together, these reports suggest that LMWHA induces inflammation via activation of TLR receptors and initiation of MyD8/NFκB signaling, which leads to production of proinflammatory cytokines and chemokines.

In physiological conditions, the activation of immune system cells is crucial for proper wound healing. In acute wounds, small hyaluronan fragments accumulating at the site of injury activate the immune system to manage disruptions in tissue integrity; however, in chronic wounds, a constant excessive inflammatory response actually prevent that wound from healing. [Id.]

LMWHA, which has strong antioxidant properties, has shown protective effects against ROS both in vitro and in vivo, and inhibits lipid peroxidation and scavenges free radicals. [Id., citing Ke, C. et al. Food Chem. Toxicol. (2011) 49 (10): 2670-75].

The anti-inflammatory potential of HMWHA has been well documented in osteoarthritis. Intra-articular injection of HMWHA has been used to treat osteoarthritis, since HA is a basic component of normal synovial fluid and the concentration of HA is decreased in osteoarthritis affected joints. For example, HMWHA was able to inhibit IL-1β expression in synoviocytes in a rabbit model of osteoarthritis. [Id., citing Miki, Y. et al. Inflamm. Res. (2010) 59 (6): 471-77]. IL-1 dependent expression of MMP-1 and MMP-3 was reduced in human synoviocytes by HMWHA treatment. In a large study, the influence of HMWHA on gene expression of various inflammatory cytokines by human fibroblast-like synoviocytes (FLS) in patients with early-stage osteoarthritis was analyzed. [Id., citing Wang, C T, et al. osteoarthritis Cartilage (2006) 14 (12): 1237-47] They reported the downregulation of IL-8 and iNOS gene expression in unstimulated FLS and aggrecanase-2, and tumor necrosis factor alpha (TNFα) gene expression in IL-1 stimulated FLS. Blocking the CD44 receptor with anti-CD44 antibody inhibited the down-regulatory effects of HMWHA on gene expression. [Id., citing Bourguignon, L Y et al. Cytoskeleton (Hoboken) (2011) 68 (12): 671-93].

The exact mechanism in which HMWHA interacts with TLR receptors, leading to inhibition of inflammatory cascades is not known. HWHA was able to significantly diminish TLR4, TLR2, MyD88 and NF-κB expression in synoviocytes in a murine model of osteoarthritis. [Id., citing Campo, G M et al. Biochim. Biophys. Acta (2011) 1812 (9): 1170-81]. They also observed reduced mRNA expression and protein production for TNFα, IL-1β, IL-17, MMP-13 and inducible nitrous oxide synthase gene in arthritic mice treated with HMWHA [Id., citing Campo, G M et al. Biochim. Biophys. Acta (2011) 1812 (9): 1170-81], but only when HWHA was administered in an early inflammatory phase of osteoarthritis.

Examples of antioxidant effects of HMWHA include a decrease in ultraviolet B-induced apoptosis and EDTA-induced oxidative damage of DNA [Id., citing Bourguignon, L Y et al. J. Biol Chem. (1997) 272 (44): 27913-27918; Pauloin, T. et al., Mol. Vis. (2009) 15: 577-83], and a decrease in apoptosis and oxidative stress triggered with benzalkonium chloride and sodium lauryl sulfate detergents which are widely used in ophthalmic preparations [Id., citing Pauloin, T. et al. Cornea (2009) 28 (9): 1032-41; Pauloin, T. et al. Eur. J. Pharm. Sci. (20008) 34 (4-5): 263-73]. The mechanisms by which HMWHA reduces oxidative stress are still not understood.

Numerous studies have shown that HA signaling plays a role in angiogenesis regulation, mainly by influencing endothelial cell behavior. Indeed, both HMWHA and LMWHA are potent regulators of angiogenesis. LMWHA stimulates vascular EC proliferation, migration, and tubule formation in vitro, as well as in various in vivo models of angiogenesis, while HMWHA displays antiangiogenic properties by inhibiting EC proliferation motility and sprout formation. [Id., citing Toole, B P. Nat. Rev. Cancer (2004): 4(7): 528-39]. The exact molecular mechanism determining the proangiogenic or antiangiogenic effects of different HA forms has not been fully elucidated. A context-dependent response to different forms of HA and a possible role of the microenvironment in this process has been suggested. In wound closure assays, adding CXCL12 to the culture medium significantly increased cell migration and induced faster wound closure. This effect was statistically augmented when cells were preincubated with HMWHA. [Id., citing Fuchs, K. et al. Cell Death Dis. (2013) 4: e819]. In vitro studies have shown that CXCR4 activation by CXCL12 was significantly increased in HUVECs pretreated with HMWHA, whereas preincubation with LMWHA blocked CXCL12 signaling in these cells.

According to some embodiments, a sodium hyaluronate product (e.g., HMWHA, LMWHA, or both) is produced by biofermentation in the strain Steptococcus equi sub. zooepidemicus. According to some embodiments, the sodium hyaluronate product is obtained from a commercial source (e.g., Bloomage Biotechnology Corp., Ltd., Jinan, China). According to some embodiments, the main components of the culture medium include: wheat peptone, yeast extract powder, and glucose

According to some embodiments, the molecular weight of the HMWHA ranges from 1,000-1,800 kDa, inclusive, i.e., at least 1000, 1100, 1200, 1300, 1400, 1500, 1600, 1700, or 1800 kDa. According to some embodiments, the molecular weight of the LMWHA is less than 10 kDa, i.e., at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, at least 9 kDa to less than 10 kDa.

According to some embodiments, the finished product comprises from about 0.10 to about 0.50 wt %, inclusive of the LMWHA, i.e., 0.10 wt %, 0.15 wt %. 0.20 wt %, 0.25 wt %, 0.30 wt %, 0.35 wt %, 0.40 wt %, 0.45 wt %, or 0.50 wt % LMWHA, and from about 0.50 to about 1.50 wt % HMWHA, inclusive, i.e., 0.50 wt %, 0.60 wt %, 0.70 wt %, 0.80 wt %, 0.90 wt %, 1.0 wt %, 1.2 wt %, 1.2 wt %, 1.3 wt %, 1.4 wt %, or 1.5 wt % HMHWA.

According to some embodiments the ratio of HMWHA to LMWHA ranges from 1:0.7 to 1:1, inclusive, i.e., 1:0.7, 0.8, 0.9 or 1.0.

### Botanical Ingredient Component

According to some embodiments, the botanical ingredient component comprises a cannabinoid.

Cannabinoids are terpenophenolic secondary metabolites produced by Cannabis. [Fischedick et al., Phytochemistry 71:2058-73 (2010)]. Cannabis strains that are used produce cannabinoids are dioecious (meaning having the male and female reproductive organs in separate individuals), with cannabinoids particularly accumulating on the unfertilized female inflorescence (meaning the complete flower head of a plant including stems, stalks, bracts, and flowers). [Ritchens et al., PLoS ONE (2018) 13:e0201119]. However, synthesis and accumulation of cannabinoids occurs in trichomes on the surfaces of not only inflorescences, but on the leaves as well. Happyana et al., Phytochemistry 87:51-59 (2013). Cannabinoids also are found in low amounts in plant seeds, roots, and pollen. [Andre et al., Front. Plant Sci. 7:19, oi:10.3389/fpls.2016.00019 (2016)]. Cannabinoids have also been found in plants from the Radula and Helichrysum genera. [Appendino et al., J. Nat. Prod. 71:1427-30 (2008)).

More than 140 different cannabinoids have been reported, although some of these are breakdown products, and they are generally classified into 11 subclasses. [Berman et al., Sci. Rep. 8:14280 (2018)]. The predominant compounds are Δ⁹-tetrahydrocannabinolic acid (THCA), cannabidiolic acid (CBDA), cannabinolic acid (CBNA), cannabigerolic acid (CBGA), cannabichromenic acid (CBCA), and cannabinodiolic acid (CBNDA). THCA is the major cannabinoid in the drug-type Cannabis, while CBDA predominates in fiber-type hemps.

The cannabis plant contains more than 60 different active synthetic ligands for CB1 and CB2, with A9-THC being the major psychoactive molecule among them. [Kendall, D A, Yudowski, G A, Frontiers Cellular Neurosci. (2017) 10: 294]. Exemplary cannabinoids include, but are not limited to, the Cannabichromenes (e.g., Cannabichromene (CBC), Cannabichromenic acid (CBCA), Cannabichromevarin (CBCV), Cannabichromevarinic acid (CBCVA)), Cannabicyclols (e.g., Cannabicyclol (CBL), Cannabicyclolic acid (CBLA), Cannabicyclovarin (CBLV)), Cannabidiols (e.g., Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA)), Cannabielsoins (e.g., Cannabielsoic acid B (CBEA-B), Cannabielsoin (CBE), Cannabielsoin acid A (CBEA-A)), Cannabigerols (e.g., Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA)), Cannabinols and cannabinodiols (e.g., Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV)), Cannabitriols (e.g., 10-Ethoxy-9-hydroxy-delta-6a-tetrahydrocannabinol, 8,9-Dihydroxy-delta-6a-tetrahydrocannabinol, Cannabitriol (CBT), Cannabitriolvarin (CBTV)), Δ8-tetrahydrocannabinols (e.g., Δ8-tetrahydrocannabinol (Δ8-THC), Δ8-tetrahydrocannabinolic acid (Δ8-THCA)), Δ9-tetrahydrocannabinols (e.g., Δ9-tetrahydrocannabinol (THC), Δ9-tetrahydrocannabinol-C4 (THC-C4), Δ9-tetrahydrocannabinolic acid A (THCA-A), Δ9-tetrahydrocannabinolic acid B (THCA-B), Δ9-tetrahydrocannabinolic acid-C4 (THCA-C4), Δ9-tetrahydrocannabiorcol (THC-C1), Δ9-tetrahydrocannabiorcolic acid (THCA-C1), Δ9-tetrahydrocannabivarin (THCV), Δ9-tetrahydrocannabivarinic acid (THCVA)), and miscellaneous cannabinoids (e.g., 10-Oxo-Δ6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Δ9-cis-tetrahydrocannabinol (cis-THC), tryhydroxy-Δ9-tetrahydrocannabinol (triOH-THC), 3,4,5,6-tetrahydro-7-hydroxy-α-α-2-trimethyl-9-n-propyl-2,6-methano-2H-1-benzoxocin-5-methanol (OH-iso-HHCV)).

The cannabinoid profile of a Cannabis plant, and relatedly a plant's CBD/THC ratio, is primarily dependent on the plant's genetic background and that each individual plant invariably belongs to its distinct chemical group throughout its life cycle. [Beutler et al., Econ. Bot. 32:387-94 (1978)].

In addition to the cannabinoids, Cannabis are also rich in bioactive terpenoids. Terpenoids or terpenes are aromatic compounds synthesized in trichomes. These compounds normally occur in several plant species, including Cannabis sativa, Mirabilis jalapa, Lithophragm glabrum, Cordia verbenacea, Eucalyptus globus, Syzygium aromaticum, Senna didymobotrya, Cymbopogon citratus, Pterodon emarginatus, Artemisia campestris, Lantana camara, Centella asiatica, Cyanthillium cinereum, Croton bonplandianus, and Citrus limon. [Goncalves et al., Molecules 25:1567 (2020)].

For example, B-caryophyllene and α-caryophyllene are the major sesquiterpenes of Cannabis. Booth et al., PLos ONE 12:e0173911 (2017). Caryophyllenes are phytocannabinoids with strong affinity to CB2 but not CB₁. Goncalves et al. (2020). Caryophyllenes have been reported to be repellent, antimicrobial, antibacterial, anticancer, antiproliferative, antifungal, AChE inhibitors, antioxidant, anti-inflammatory. [Fidyt et al., Cancer Med. 5:3007-17 (2016); Sabulal et al., Phytochemistry 67:2469-73 (2006); Su et al., Nat. Prod. Commun. 11:845-48 (2016); Sarvmeili et al., Res. Pharm. Sci. 11:476-83 (2016); Memariani et al., Oncol. Lett. 11:1353-60 (2016); Segat et al., Neuropharmacology 125:207-19 (2017); Bento et al., Am. J. Pathol. 178:1153-66 (2011); Gertsch et al., Proc. Natl. Acad. Sci. USA 105:9099-9104 (2008); Alberti et al., J. Ethnopharmacol. 155:485-94 (2014)].

Limonene ((4R)-1-methyl-4-prop-1-en-2-ylcyclohexene) is the most common natural monoterpene found in nature and is found in Cannabis sativa oilseed and in orange, lemon, and tangerine oils. Araujo-Filho et al., Neuroscience 358:158-69 (2017). Limonene does not interact with either CB₁ or CB₂. Santiago et al., Cannabis Cannabinoid Res. 4:165-76 (2019). Limonene has been reported to be anti-inflammatory, gastro-protective, anti-nociceptive, anti-tumor, neuroprotective, anti-hyperalgesic, anti-depressive, and anxiolytic. [Araujo-Filho et al. (2017); Al-Ghezi et al., Front. Immunol. 10:1921 (2019); Sun, Altern. Med. Rev. J. Clin. Ther. 12:259-64 (2007); Shah et al., Anim. Models Exp. Med. 1:328-33 (2018); d'Allessio et al., Life Sci. 92:1151-56 (2013); de Almeida et al., Inflammation 40:511-22 (2017); do Amaral et al., Biol. Pharm. Bull. 30:1217-20 (2007); Piccinelli et al., Nutr. Neurosci. 18:217-24 (2015)].

Linalool (3,7-dimethylocta-1,6-dien-3-ol) is a monoterpene compound present in several medicinal plants and fruits, including Cannabis sativa, and is used in cosmetics and flavoring ingredients. Zhang et al., Enzym. Microb. Technol. 134:109462 (2020). Linalool has been reported to be anti-inflammatory, anticancer, anxiolytic, neuroprotective, UV-protective, and pain reductive. Kim et al., Int. Immunopharmacol. 74:105706 (2019); Sabogal-Guaqueta et al., Biomed. Pharmacother. 118:109295 (2019); Harada et al., Front. Behav. Neurosci. 12:241 (2018); Xu et al., Life Sci. 174:21-27 (2017); Iwasaki et al., World J. Gastroenterol. 22:9765-74 (2016); Gunsaeelan et al., Photochem. Photobiol. Sci. Off. J. Eur. Photochem. Assoc. Eur. Soc. Photobiol. 15:851-60 (2016); Katsuyama et al., Biomed. Res. 33:175-81 (2012).

Terpineol (2-(4-methylcyclohex-3-en-1-yl)propan-2-ol) is a volatile monoterpene found in Cannabis sativa as well as cajuput, pine, and petitgrain oils. Gonçalves et al. (2020). Terpineol has been reported to be antinociceptive, antifungal, anti-inflammatory, antidiarrheal, pain reductive, memory enhancing, algeacidic, insect repellent, anti-proliferative, and anticancer. de Oliveira et al., Chem. Biol. Interact. 254:54-62 (2016); Chaudhari et al., Food Chem. 311:126010 (2020); de Oliveira et al., Basic Clin. Pharmacol. Toxicol. 111:120-25 (2012); dos Santos Negreiros et al., Biomed. Pharmacother. 110:631-40 (2019); Gouveia et al., Biomed. Pharmacother. 105:652-61 (2018); Parvardeh et al., Iran. J. Basic Med. Sci. 19:201-08 (2016); Kim et al., Biosci. Biotechnol. Biochem. 70:1821-26 (2006); Nogueira et al., Inflamm. Res. 63:769-78 (2014); Jing et al., Bot. Stud. 56:35 (2015); Chen et al., Ecotoxicol. Environ. Saf. 167:435-40 (2019); Wua et al., Nat. Prod. Commun. 9:1515-18 (2014); Villa-Ruano et al., Chem. Biodivers. 15:e1800354 (2018); Hassan et al., Anticancer Res. 30:1911-19 (2010).

γ-terpinene (1-methyl-4-propan-2-ylcyclohexa-1,4-diene) is a monoterpene structurally similar to 1.8-cineol (eucalyptol) and is found in the essential oils of Cannabis sativa and other plants including the Eucalyptus genus (Myrtaceae), Cupressus cashmeriana, Lippia microphylla, Lavandula angustifolia, and Citrus myrtifolia. Gonçalves et al. (2020). γ-terpinene has been reported to be anti-inflammatory, antimicrobial, analgesic, and anticancer. Djenane et al., Food Sci. Technol. Int. 17:505-15 (2011); Ramalho et al., Planta Med. 81:1248-54 (2015); da Silva Lima et al., Eur. J. Pharmacol. 699:112-17 (2013); Guimaraes et al., Phytother. Res. PTR 27:1-15 (2013); Siveen et al., Can. J. Physiol. Pharmacol. 89:691-703 (2011); Ramalho et al., Axis. Planta Med. 82:1341-45 (2016); Baldissera et al., Exp. Parasitol. 162:43-48 (2016); Assmann et al., Biomed. Pharmacother. 103:1253-61 (2018).

α-pinene is found not only in Cannabis sativa but also in essential oils of many aromatic plants, such as Lavender angustifolia, Rosmarinus officinalis, and coniferous trees. Begum et al., Acta Sci. Polonorum. Technol. Aliment. 12:61-73 (2013). α-pinene has been reported to be antioxidant, antimicrobial, anti-tumor, hypnotic, anxiolytic, neuroprotective, cytoprotective, and antinociceptive. Zhao et al., Chemotherapy 63:1-7 (2018); Ibrahim et al., Planta Med. 85:431-38 (2019); Nissen et al., Fitoerapia 81:413-19 (2010); Yang et al., Mol. Pharmacol. 90:530-39 (2016); Satou et al., Phytother. Res. PTR 28:1284-87 (2014); Mercier et al., Int. J. Occup. Med. Environ. Health 22:331-42 (2009); Karthikeyan et al., Life Sci. 212:150-58 (2018); Karthikeyan et al., Life Sci. 217:110-18 (2019); Bouzenna et al., Biomed. Pharmacother. 93:961-6 (2017).

β-pinene is found in many plants essential oils and can be obtained commercially by distillation or by α-pinene conversion. Iseppi et al., Molecules 24:2302 (2019). β-pinene has been reported to be antimicrobial, antioxidant, anti-immobility, and anti-adhesive. Mahajan et al., Environ. Sci. Pollut. Res. Int. 26:456-63 (2019); Guzman-Gutierrez et al., Life Sci. 128:24-29 (2015); Astani et al., Iran. J. Microbiol. 6:149-55 (2014); de Macedo Andrade et al., Curr. Top. Med. Chem. 18:2481-90 (2018); Jia et al., Antimicrob. Agents Chemother. 46:947-57 (2002); da Silva et al., Molecules 17:6035-16 (2012).

β-elemene (1-methyl-1-vinyl-2,4-diisopropenyl-cyclohexane) is a derivative, which may arise due to oxidation or due to thermal- or UV-induced rearrangements during processing or storage. Booth et al. (2017). β-elemene has been reported to be anticancer. Deng et al., Phytother. Res. PTR 33:2248-56 (2019); Wu et al., Exp. Ther. Med. 13:3153-57 (2017); Fang et al., Oncol. Lett. 15:3957-64 (2018); Cai et al., Oncol. Lett. 16:6019-25 (2018); Liu et al., Oncol. Rep. 32:2635-47 (2014); Li et al., Anticancer Res. 33:65-76 (2013); Wei et al., Oncol. Rep. 37:3159-66 (2017); Yoshida et al., Lab. Investig. J. Tech. Methods Pathol. 93:1184-93 (2013); Liu et al., Biomed. Pharmacother. 95:1789-98 (2017); Zhang et al., Int. Immunopharmacol. 10:738-43 (2010).

β-ocimene (3,7-dimethyl-1,3,6-octatriene) is acyclic monoterpene that serves as a chemical cue to attract natural enemies of phytophagous insect in several plant species. Booth et al. (2017). β-ocimene has been reported to be antitumor, antifungal, and anticonvulsant. Bomfim et al., Basic Clin. Pharmacol. Toxicol. 118:208-13 (2016); Sayyah et al., J. Enthnopharmacol. 94:283-87 (2004).

Camphene (2,2-dimethyl-3-methylidenebicyclo(2.2.1)heptane) is a cyclic monoterpene present in Cannabis inflorescence in low titer but abundant in Thymus vulgaris oil. Gonsalves et al. (2020). Camphene has been reported to be expectorant, spasmolytic, and antimicrobial. Baser et al., Handbook of Essential Oils: Science, Technology, and Applications; CRC Press/Taylor & Francis: Boca Raton, Fla. (2010); Feng et al., Environ. Sci. Pollut. Res. Int. 26:16157-65 (2019); Benelli et al., Ecotoxicol. Environ. Saf. 148:781-86 (2018); Benelli et al., Environ. Sci. Pollut. Res. Int. 25:10383-91 (2018).

Nerolidol ((6E)-3,7,11-trimethyldodeca-1,6,10-trien-3-ol; peruviol) is a noncyclic sesquiterpene alkene alcohol common to citrus peels, Piper claussenianum, Baccharis dracunculifolia, and Cannabis. Baldissera et al., Naunyn-Schmiedeberg's Arch. Pharmacol. 391:753-59 (2018). Nerolidol has been reported to be antimicrobial and anti-inflammatory. Alonso et al., Biochim. Biophys. Acta Biomembr. 1861:1049-56 (2019); Zhang et al., Phytother. Res. PTR 31:459-65 (2017); Iqubal et al., 236:116867 (2019); Iqubal et al., Eur. J. Pharmacol. 863:172666 (2019).

Euphol, a tetracyclic triterpene and a minor Cannabis component, is usually extracted in alcoholic preparations. Pellati et al., Biomed. Res. Int. 2018:1691428 (2018). Euphol has been report to be anticancer and anti-inflammatory. Betancur-Galvis et al., Mem. Inst. Oswaldo Cruz 97:541-46 (2002); Prinsloo et al., J. Ethnopharmacol. 210:133-55 (2018); Mazior et al., Fur Naturforschung. Cjournal Biosci. 66:360-66 (2011); Silva et al., Exp. Ther. Med. 16:557-66 (2018); Cruz et al., Phytomedicine: Int. J. Phytother. Phytopharm. 47:105-112 (2018); Wang et al., Mol. Med. Rep. 8:1279-85 (2013); Silva et al., Investig. New Drugs 37:223-37 (2019).

In the plant, the terpenoid compounds synthesized alongside phytocannabinoids are important volatile constituents that are responsible for the plant's characteristic smell and also serve for different organic functions, such as insect repellent, repellent to herbivore attack, and attractive to pollinators. Cannabis terpenoid composition provides information about the origin of the plant, as unique chemical abundances of specific terpenoids are predicted to be associated with chemotypes and species level taxa. Fischedick et al. (2010). Analysis of 72 Cannabis strains showed that the total terpenoid content ranged between 0.6 and 3.3%, while the total cannabinoid content ranged between 12.6±31.5%. [Ritchens et al. (2018) PLoS ONE (2018) 13:e0201119].

Numerous methods are known to extract cannabinoids and terpenoids from Cannabis. Sonication is the most common and is the method recommended by both United Nations Office on Drugs and Crime (UNDOC) and the American Herbal Pharmacopoeia (AHP). Giese et al., J. AOAC Int. 98:1503-22 (2015). Methods in the AHP monograph recommend drying and powdering the sample first and require a separate moisture determination to accurately assess the content of the initial inflorescence. It is also noted that such drying and powdering alters the terpenoid content. Giese et al. (2015); Swift et al., PLoS ONE 8:e70052 (2013). There are three notable limitations of sonication: injection of Cannabis extracts for analysis by gas chromatography typically results in decarboxylation in the injection port, and consequently it is only the decarboxylated phytocannabinoids that are measured directly by these techniques; while derivatization of the metabolite extract via silylation enables the measurement of both acid and neutral phytocannabinoids, a complete derivatization yield is difficult to obtain and thus quantification is less reliable; and it has been suggested that phytocannabinoids may thermally degrade (oxidize, isomerize) in the injector port and column. Berman et al. (2018).

Most cellular cannabinoid effects are mediated by two G protein-coupled receptors (GPCRs), CB₁ and CB₂. CB₁ receptors are present in very high levels in the brain and in lower amounts in a more widespread fashion, and they mediate most psychoactive effects of cannabinoids. CB₂ receptors are more limited in distribution, being found in certain immune cells and neurons. Mackie, J. Neuroendocrinol. 20(Supp. 1):10-14 (2008). Cannabinoids mediate both inhibitory and stimulatory effects on the immune system by modulating cytokine expression. [Raduner, S. et al., J. Biol. Chem. (2006) 281 (20): 14192-14206, citing Klein, T., et al. J. Leukocyte Biol. (2003) 74: 486-96; Croxford, J. L. and Yamamura, t. J. Neuroimmunol (2005) 166: 3-18]. Other GPCRs, ion channels, and nuclear receptors also interact with cannabinoids. [Zou et al., Int. J. Mol. Sci. 19:833 (2018)]. N-arachidonoyl-ethanolamine and 2-arachidonoylglycerol are endogenous agonists of cannabinoid receptors. Zou et al. (2018). A putative third CB receptor, GPR55, shares only 13.5% sequence identity to CB₁ and 14.4% sequence identity to CB₂. [Lauckner et al., Proc. Natl. Acad. Sci. USA 105:2699-2704 (2008)]. GPR55 shares some ligands with the CB₁ and CB₂, it has additional agonist ligands with novel chemotypes. [Kotsikorou et al., Biochemistry 52:9456-69 (2013)].

In addition to the cannabinoids, endogenous agonists for CB₁ and CB₂ (and in some cases GPR55) include arachidonoyl ethanolamide (anandamide), 2-arachidonoyl glycerol, and 2-arachidonyl glyceryl ether (noladin ether). [Pertwee et al., Prostaglandins Leukot. Essent. Fatty Acids 66:101-21 (2002)]. There are also three classes of synthetic agonists: classical, which are similar to THC; bicyclic; and aminoalkylindole cannabinoids. [Hourani et al., Brain Neurosci. Adv. 2:1-8 (2018)]. Many of these synthetic cannabinoids are far more potent than THC, and they also display greater efficacy. Selective, synthetic agonists for CB₁ and CB₂ include SR141716A, LY320135, SR144528, 6-iodopravadoline (AM630), Nabilone, CP55940, and R-(+)-WIN55212-2. Pertwee et al. (2002); Hourani et al. (2018). SR141716A and LY320135 are highly selective for CB₁, and SR144528 and AM630 are highly selective for CB₂. Pertwee et al. (2002).

Both phytocannabinoids and synthetic cannabinoids can directly impact the endocannabinoid system via a variety of pharmacological mechanisms, including agonism, antagonism, and allosteric modulation [see Bonn-Miller et al., Int. Rev. Psychiatry (2018) 30:277-84].

Oral formulations of synthetic cannabinoids are also available commercially. For instance, Nabilone is a synthetic cannabinoid marketed as CESAMET® in Canada, the United States, the United Kingdom, and Mexico. Nabilone is formulated as capsules suitable for oral administration. NAMISOL® is approved for use as an antiemetic and analgesic for neuropathic pain. SAVITEX® is a mouth spray containing THC and CBD. U.S. Pat. No. 8,808,734. It is approved for the treatment of spasticity due to multiple sclerosis and as adjunct analgesic in advanced cancer patients. [Paudel et al., Drug Dev. Indus. Pharm. 36:1088-97 (2010)]. Administration of synthetic cannabinoid formulations show fewer undesirable side effects than THC. See U.S. Pat. No. 8,808,734.

Bioavailability of pharmaceutical substances taken orally depends on the extent to which the pharmaceutically active substance is absorbed from the intestinal environment across the intestinal mucosa. Lipophilic pharmaceutical substances are generally poorly absorbed from the intestinal environment, inter alia because of their poor solubility and/or dispersibility in water. Bioavailability of a pharmaceutical substance taken orally furthermore depends on the susceptibility of the substance to the so-called first pass effect. Substances absorbed from the intestine, before being distributed throughout the body, must pass the liver first where they may be metabolized immediately. CBD is generally assumed to be rather susceptible to first-pass liver metabolization. Oral bioavailability of CBD is low and unpredictable, and CBD is unstable. [Zhornitsky et al., Pharmaceuticals (2012) 5:529-52; Poortman et al., Forensic Sci. Int. (1999) 101:1-8].

Cannabinoids are lipophilic substances that are known to be poorly soluble in water (less than 1 μg/mL). As an example, CBD is soluble in ethanol (36 mg/mL) and dimethylsulfoxide (DMSO) (60 mg/mL). CBD is highly lipophilic and subject to first-pass metabolism after oral dosing. [Nichols, J M, Kaplan, B L F. Cannabis and Cannabinoid Res. (2018) doi: 10.1.089/can.2018.0073, citing Samara, E. et al. Drug Metab. Dispos. (1988) 16: 469-72]. Data to date overwhelmingly demonstrate that CBD is immunosuppressive and anti-inflammatory. [Id.] Identification of receptors through which CBD acts in the immune system and the cell types on which those receptors are expressed that mediate the CBD effects remain unclear. [Id.] It is known that effects of CBD are mediated through activation of CB1, CB2, transient receptor potential V1 (TRPV1), known as the vanilloid receptor, adenosine A2A receptors, and PPAR-γ receptors, blockade of GPR55 receptors, and fatty acid amide hydrolase (FAAH) inhibition. [Id.] The effects of CBD on immune responses can involve innate or adaptive responses. [Id.] Targets of suppression include cytokines, such as TNF-α, IFN-γ, Il-6, IL-1β, IL-2, IL-17A, and chemokines, such as CCL-2. [Id.] The overall mechanism of CBD involves direct suppression of target cells, such as effector T cells, innate, and microglial cells, through suppression of kinase cascades and various transcription factors.

However, limited data is available examining CBD's effects on various T cell subsets. While results suggest that B cells can be targets of suppression by CBD, there are only a few studies in which B cells are identified as targets of CBD. Direct suppression of target cells also includes induction of IκB, which could contribute to decreased NF-κB activity. [Id.] The involvement of regulatory cell induction by CBD is also a major part of the mechanism by which CBD controls immune responses, and CBD has been shown to induce regulatory T cells (Tregs) and myeloid-derived suppressor cells (MDSCs), the latter of which are innate, myeloid cells that possess the ability to suppress T cell responses [Id.; see Gabrilovich, D., Nagaraj, S. Nat. Rev. Immunol. (2009) 9 (3): 162-74]. CBD-induced apoptosis is likely a mechanism in many target cells. Nichols, J M, Kaplan, B L F. Cannabis and Cannabinoid Res. (2018) doi: 10.1.089/can.2018.0073]

EPIDIOLEX® is the first FDA approved CBD pharmaceutical (Greenwich Biosciences Inc., Carlsbad, Calif.), with approved use in patients two years and older with Dravet syndrome or Lennox-Gastaut syndrome. Four randomized, double-blind, multicenter clinical trials evaluated the use of CBD in patients with Dravet syndrome or Lennox-Gastaut syndrome regarding the efficacy and safety in convulsive and drop seizure, respectively. All trials demonstrated a significant absolute reduction in seizure frequency. Devinsky et al., N. Engl. J. Med. (2017) 376:2011-20; Devinsky et al., N. Eng. J. Med. (2018) 378:1888-97; Thiele et al., Lancet (2018) 1085-96].

According to some embodiments, the cannabinoid is derived from hemp. According to some such embodiments, the cannabinoid is nonpsychoactive. According to some embodiments, the cannabinoid is CBD. According to some embodiments, the cannabinoid is not a full spectrum CBD, i.e., it does not contain all the cannabinoids found in the cannabis plant in nature. According to some embodiments, the cannabinoid is a decarboxylated CBD (see Formula IX below).

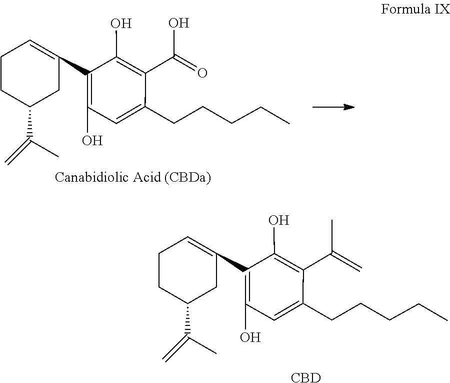

According to some embodiments, the isolate comprises about 20% decarboxylated CBD. According to some embodiments, the isolate comprising about 20% decarboxylated CBD is in the form of a THC free nano-infused water soluble powder. According to some embodiments, the isolate comprising about 20% decarboxylated CBD is obtained from a commercial source (e.g., Global Cannabinoids, Las Vegas, Nev.). According to some embodiments, the isolate comprising about 20% CBD comprises <1% cannabidivarin (CBDV), which has a similar molecular structure to CBD, but instead of having a pentyl chain, it has a propyl chain. According to some embodiments, decarboxylation is by heat not to exceed 300° F., e.g., 200° F. (93° C.) for 75 min, 225° F. (107° C.) for 50 min, or 250° F. (121° C.) for 30 min.

An exemplary analysis of a decarboxylated CBD in the form of a THC free nano-infused water-soluble powder is shown below (LOQ=limit of quantitation).

| Analyte | LOQ % | Mass % | Mass mg/g |
|---|---|---|---|
| THCa | 0.010 | <LOQ | <LOQ |
| Δ9-THC | 0.010 | <LOQ | <LOQ |
| CBDa | 9.010 | <LOQ | <LOQ |
| CBD | 0.102 | 18.218 | 18.218 |
| CBC | 0.005 | <LOQ | <LOQ |
| CBG | 0.005 | <LOQ | <LOQ |
| CBN | 0.010 | <LOQ | <LOQ |
| THCV | 0.005 | <LOQ | <LOQ |
| Δ8-THC | 0.005 | <LOQ | <LOQ |
| CBGa | 0.005 | <LOQ | <LOQ |
| CBDV | 0.005 | 0.051 | 0.051 |
| Total | | 18.269 | 18.269 |

The reported result is based on a sample weight with the applicable moisture content for that sample; unless otherwise stated all quality control samples performed within specifications established by the Laboratory.

According to some embodiments, total CBD can be represented by the formula CBDa*0.877+CBD, and total THC can be represented by the formula THCa*0.877+Δ9-THC+Δ8THC. According to some embodiments, the powder contains one or more inactive ingredients, e.g., tapioca maltdextrin.

According to some embodiments, the finished product comprises from 1.0 wt % to about 5.0 wt %, inclusive of the 20% CBD-THC-free nanoinfused water-soluble powder, i.e., at least 1.0 wt %, at least 1.25 wt %, at least 1.5 wt %, at least 1.75 wt %, at least 2.0 wt %, at least 2.25 wt %, at least 2.5 wt %, at least 2.75 wt %, at least 3.0 wt %, at least 3.25 wt %, at least 3.5 wt %, at least 3.75 wt %, at least 4.0 wt %, at least 4.25 wt %, at least 4.5 wt %, at least 4.75 wt %, or at least 5 wt %.

### Cosmetic Composition Stabilizing System

According to some embodiments, the cosmetic composition stabilizing system comprises an effective amount of an arginine component. According to some embodiments, the arginine component comprises i) arginine, or a conjugate, or an analog thereof, ii) an organic acid, a conjugate, or an analog thereof, and iii) a solvent. As used herein, the phrase “arginine, a conjugate, or an analog thereof” is referred to as an “arginine compound.” As used herein, the phrase “organic acid a conjugate, or a derivative thereof” is referred to as an “organic acid compound.” According to some embodiments the arginine component comprises i) an arginine compound, ii) an organic acid compound, and iii) a solvent.

According to some embodiments, the cosmetic composition stabilizing system has a preservative effect on the composition, a solubilizing effect, a stabilizing, a neutralizing, or anti-microbial effect on the composition, a moisturizing, and/or healing effect, or any combination thereof. For example, according to some embodiments the cosmetic composition stabilizing system has a preservative effect on the composition and/or a solubilizing effect on the active agent. According to some embodiments the cosmetic composition stabilizing system has a preservative effect on the composition, and/or a solubilizing effect on the active agent, and/or a stabilizing effect on the composition. According to some embodiments, the cosmetic composition stabilizing system has a preservative effect on the composition, and/or a solubilizing effect on the active agent, and/or a stabilizing effect and/or a neutralizing effect on the composition. According to some embodiments, the cosmetic composition stabilizing system has a preservative effect on the composition, and/or a solubilizing effect on the active, and/or a stabilizing, neutralizing, and/or anti-microbial effect on the composition. According to some embodiments, the cosmetic composition stabilizing system has a preservative effect on the composition, and/or a solubilizing effect on the active, a stabilizing, and/or a neutralizing, and/or an anti-microbial effect on the composition. According to some embodiments, the cosmetic composition stabilizing system has a preservative effect on the composition, a solubilizing effect on the active, and/or a stabilizing, a neutralizing, and/or an anti-microbial effect on the composition and/or a moisturizing cosmetic/therapeutic effect. According to some embodiments, the cosmetic composition stabilizing system has a preservative effect on the composition, and/or a solubilizing effect on the active, a stabilizing, neutralizing, and/or anti-microbial effect on the composition, and/or a moisturizing cosmetic/therapeutic effect. According to some embodiments, the cosmetic composition stabilizing system has a preservative effect on the composition, and/or a solubilizing effect on the active, and/or a stabilizing, a neutralizing, and/or an anti-microbial effect on the composition, and/or a moisturizing cosmetic/therapeutic effect. According to some embodiments, the cosmetic composition stabilizing system has a preservative effect on the composition, and/or a solubilizing, stabilizing, neutralizing, and/or anti-microbial effect on the composition, and/or a moisturizing, exfoliating and/or healing cosmetic/therapeutic effect.

According to some embodiments, the arginine compound is arginine, or a salt, conjugate, or analog thereof. For example, the arginine may be D,L-arginine, D-arginine, L-arginine, alkyl (ethyl, methyl, propyl, isopropyl, butyl, isobutyl, t-butyl) esters of arginine and conjugates and analogs thereof.

According to some embodiments, the arginine conjugate may be a conjugate acid or a conjugate base. For example, the arginine conjugate may be argininum, or argininate.

According to some embodiments, the arginine compound may be employed in any suitable amount. For example, the arginine compound may be present in the arginine component in at least about 1.0 wt %, at least about 5.0 wt %, at least 10.0 wt %, at least about 15.0 wt %, at least 20.0 wt %, at least 25.0 wt %, at least 30.0 wt %, at least 35.0 wt %, at least 40.0 wt %, at least 45.0 wt %, at least 50.0 wt %, at least 55.0 wt %, at least 60.0 wt %, at least 65.0 wt %, at least 70.0 wt %, at least 75.0 wt %, at least 80.0 wt %, at least 85.0 wt %, at least 90.0 wt %, and about 95.0 wt % of the total weight of the arginine component. Exemplary amounts include 10.0 wt % to about 70.0 wt %, inclusive, based upon the total weight of the arginine component, about 20 wt % to about 60 wt % inclusive, based upon the total weight of the arginine component, about 30 wt % to about 50 wt % inclusive of the total weight of the arginine component.

According to some embodiments, the organic acid compound can be an organic acid, its conjugate, or an analog thereof. According to some embodiments the organic acid can be any suitable organic acid. For example, organic acids may be substituted and non-substituted aliphatic (saturated and unsaturated) and aromatic acids. Organic acids may possess as substituents one or more functional groups, such as alkyl, alkenyl, alkynyl, halogen, hydroxy, carbonyl, carboxylic acid, aldehyde, ester, amide, carbonate, carbamate, ether, amino, cyano, isocyano, oxy, oxo, thia, aza, azide, imine, nitro, nitrate, nitroso, nitrosooxy, cyanate, isocyanate, thiocyanate, isothiocyanate, sulfinyl, sulfhydryl, sulfonyl, phosphino, wherein each of the alkyl, alkenyl, alkynyl and amino groups may themselves be optionally substituted with one or more of the preceding functional groups. According to some embodiments, some functional groups, such as hydroxy, will impart or augment a character to the acid that is suitable for the present composition, such as a hygroscopic character. According to some embodiments, the organic acid contains multiple carboxylic acid groups.

According to some embodiments, the organic acid is a hydroxy acid. According to some embodiments, the hydroxy acid is an α-hydroxy acid (AHA), a β-hydroxy acid (BHA), a γ-Hydroxy acids (GBA), an ω-Hydroxy acids, a monohydroxy acid (MHA), a polyhydroxy acid (PHA)/polycarboxy hydroxy acid (PCHA), an aliphatic hydroxy acid (AlHA), an aromatic hydroxy acids acid (ArHAs), an arylaliphatic hydroxy acid (AAHA), a hydroxy fatty acids, and the like.

According to some embodiments, the hydroxy acid is an α-hydroxy acid (AHA). The alpha (α) carbon in organic molecules refers to the first carbon atom that attaches to a functional group, such as a carbonyl. According to some embodiments, the AHA is an alkyl AHA, arylalkyl AHA, or a polycarboxyl AHA.

According to some embodiments, the organic acid component comprises anisic acid, also known as 2-methoxybenzoic acid; o-Anisic acid; 579-75-9; o-Methoxybenzoic acid; 2-Anisic acid; 0-Methylsalicylic acid; Benzoic acid, 2-methoxy-;Salicylic acid methyl ether; 2-Methoxy-benzoic acid; 529-75-9, molecular formula C₈H₈O₃.

According to some embodiments, the organic acid component comprises levulinic acid also known as 4-Oxopentanoic acid; 123-76-2; Laevulinic acid; Pentanoic acid, 4-oxo Oxovaleric acid; Levulic acid; 3-Acetylpropionic acid; 4-Ketovaleric acid; LEVA; molecular formula C₅H₈O₃.

According to some embodiments, the organic acid component comprises mandelic acid, also known as 17199-29-0; (S)-(+)-Mandelic acid; (S)-Mandelic acid; (S)-2-Hydroxy-2-phenylacetic acid; L-mandelic acid; L-(+)-mandelic acid; S-(+)-Mandelic acid; (2S)-2-hydroxy-2-phenylacetic acid; UNII-L0UMW58G3T; l(+)-mandelic acid; molecular formula C₈H₈O₃.

According to some embodiments, the organic acid component comprises salicylic acid, also known as 2-Hydroxybenzoic acid; 69-72-7; o-hydroxybenzoic acid; 2-Carboxyphenol; o-Carboxyphenol; Rutranex; Salonil; Retarder W; Keralyt, molecular formula C7H6O3 or HOC₆H₄COOH.

According to some embodiments, the organic acid component comprises sorbic acid, also known as 110-44-1; 2,4-Hexadienoic acid; (2E,4E)-hexa-2,4-dienoic acid; 2E,4E-Hexadienoic acid; Panosorb; Sorbistat; 2-Propenylacrylic acid; trans, trans-Sorbic acid; Hexadienoic acid, molecular formula C₆H₈O₂ or CH₃CH═CHCH═CHCOOH.

According to some embodiments, the organic acid component comprises benzoic acid, also known as 65-85-0; Dracylic acid; benzenecarboxylic acid; Carboxybenzene; Benzeneformic acid; phenylformic acid; Benzenemethanoic acid; Phenylcarboxylic acid; Retardex, molecular formula C₇H₆O₂ or C₆H₅COOH.

According to some embodiments, the organic acid component comprises ferulic acid, also known as trans-4-Hydroxy-3-methoxycinnamic acid, and trans-Ferulic acid, molecular formula HOC₆H₃(OCH₃)CH═CHCO₂H.

According to some embodiments, the organic acid component comprises syringic acid, also known as 3,5-Dimethoxy-4-hydroxybenzoic acid, 4-Hydroxy-3,5-dimethoxy-benzoic acid, and Gallic acid 3,5-dimethyl ether, molecular formula HOC₆H₂(OCH₃)₂CO₂H.

According to some embodiments, the organic acid component comprises two organic acids selected from the group consisting of anisic acid, levulinic acid; mandelic acid; salicylic acid, sorbic acid, benzoic acid, ferulic acid, and syringic acid, e.g. anisic acid and levulinic acid; anisic acid and mandelic acid; anisic acid and salicylic acid, anisic acid and sorbic acid, anisic acid and benzoic acid, anisic acid and ferulic acid, anisic acid and syringic acid; levulinic acid and mandelic acid, levulinic acid and salicylic acid, levulinic acid and sorbic acid, levulinic acid and benzoic acid, levulinic acid and ferulic acid, levulinic acid and syringic acid; mandelic acid and salicylic acid, mandelic acid and sorbic acid, mandelic acid and benzoic acid, mandelic acid and ferulic acid, mandelic acid and syringic acid; salicylic acid and sorbic acid, salicylic acid and benzoic acid, salicylic acid and ferulic acid, salicylic acid and syringic acid, sorbic acid and benzoic acid, sorbic acid and ferulic acid, sorbic acid and syringic acid; benzoic acid and ferulic acid, benzoic acid and syringic acid; ferulic acid and syringic acid.

According to some embodiments, the organic acids, when used as a combination of two as exemplified above (for example, levulinic acid and anisic acid, etc.), together with glyceryl caprylate/caprate form a broad spectrum preservative effective against microbial contaminants, e.g., bacteria, yeast and mold. Without being limited to any particular theory, the stabilization system comprising glyceryl caprylate/caprate allows for greater penetration of both acids into the cell wall of a targeted organism, which means that the overall concentration of the acids in the formulation is lower, and the concentration of free acid required for full spectrum preservation of the product is reduced. This in turn increases efficacy in challenging finished formulations at a physiological pH, and reduces the need for additional ingredients in the finished formulation without extreme adjustments of the final pH of the finished formulation.

According to some embodiments, the organic acid compound includes any derivative of an organic acid thereof. For example, a derivative of an organic acid that would revert to their acid form when contacted with water, includes, without limitation, an easily hydrolyzable anhydride, mixed anhydride and ester derivatives of the organic acids.

According to some embodiments, the organic acid compound may be employed in any suitable amount. For example, the organic acid compound may be present in the arginine component as about 1.0 wt %, 5.0 wt %, 10.0 wt %, 15.0 wt %, 20.0 wt %, 25.0 wt %, 30.0 wt %, 35.0 wt %, 40.0 wt %, 45.0 wt %, 50.0 wt %, 55.0 wt %, 60.0 wt %, 65.0 wt %, 70.0 wt %, 75.0 wt %, 80.0 wt %, 85.0 wt %, 90.0 wt %, and about 95.0 wt % of the total weight of the arginine component. Exemplary amounts include about 10.0 wt %, to about 70.0 wt % based upon the total weight of the arginine component, about 20 wt % to about 60 wt % based upon the total weight of the arginine component, about 30 wt % to about 50 wt % of the total weight of the arginine component.

According to some such embodiments, the pH is high enough to keep the acid in solution and low enough to keep the glyceryl caprylate/caprate from hydrolyzing. For example, the pH of the raw material cosmetic composition stabilization system in water comprising glyceryl caprylate/caprate can range from pH 4.1-6.9, inclusive, i.e., pH 4.1, 4.2, 4.3, 4.4, 4.5, 4.6, 4.7, 4.8, 4.9, 5.0, 5.1, 5.2, 5.3, 5.43, 5.5, 5.6, 5.7, 5.8, 5.9, 6.0, 6.1, 6.2, 6.3, 6.4, 6.5, 6.6, 6.7, 6.8, 6.9. According to some embodiments, the pH of the finished product/cosmetic composition comprising glyceryl caprylate/caprate ranges from pH 6.0-6.5, inclusive, i.e., pH 6.0, 6.1, 6.2, 6.3, 6.4, 6.5.

According to some embodiments, the arginine component of the cosmetic composition stabilizing system comprises a solvent. Examples of the solvent may include water, low molecular weight alcohols such as C₁₋₆ branched or straight chain alcohols, e.g., methanol, ethanol and isopropanol, low molecular weight ketones such as C₁₋₆ branched or straight chain ketones, e.g., acetone, aromatic compounds and low molecular weight alkanes, such as C₁₋₁₀ branched or straight chain alkanes.

According to some embodiments, the arginine component comprises a polar solvent. Exemplary polar solvents include: water; alcohols (such as ethanol, propyl alcohol, isopropyl alcohol, hexanol, benzyl alcohol, polyhydric alcohol); polyols (such as propylene glycol, polypropylene glycol, butylene glycol, hexylene glycol, polyethylene glycols), sugar alcohols (e.g., malitol, sorbitol), glycerine; panthenol dissolved in glycerine, flavor oils and mixtures thereof. Mixtures of these solvents can also be used. According to some embodiments, the solvent is water.

According to some embodiments, the solvent may be employed in any suitable amount. For example, the solvent may be present in the arginine component in about 1.0 wt %, 5.0 wt %, 10.0 wt %, 15.0 wt %, 20.0 wt %, 25.0 wt %, 30.0 wt %, 35.0 wt %, 40.0 wt %, 45.0 wt %, 50.0 wt %, 55.0 wt %, 60.0 wt %, 65.0 wt %, 70.0 wt %, 75.0 wt %, 80.0 wt %, 85.0 wt %, 90.0 wt %, and about 95.0 wt % of the total weight of the arginine component. Exemplary amounts include 10.0 wt %, to about 70.0 wt %, inclusive, based upon the total weight of the arginine component, about 20.0 wt % to about 60.0 wt %, inclusive, based upon the total weight of the arginine component, about 30.0 wt % to about 50.0 wt %, inclusive, of the total weight of the arginine component.

According to some embodiments, the range of pH of the cosmetic or dermatologic formulation stabilizing system, which is within the range of pH 4.1-8.5, inclusive, i.e., pH 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, depends on the organic acids used.

According to some embodiments, the cosmetic composition stabilizing system comprising the arginine component of the present invention may be present as at least about 0.001 wt %, at least 0.005 wt %, at least 0.01 wt %, at least 0.05 wt %, at least 0.10 wt %, at least 0.50 wt %, at least 1 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt % of the total weight of the formulation.

According to some embodiments, the cosmetic composition stabilizing system comprising the arginine component of the present invention may be present in about 1.0 wt % to about 100.0 wt %, about 10.0 wt % to 70.0 wt %, or about 20.0 wt % to 50.0 wt % based on the total weight of the formulation. According to some embodiments, the arginine component of the present invention may be present in about 0.001 wt % to 10.0 wt %, about 0.1% to 5.0 wt %, or about 1.0 wt % to 3.0 wt % based on the total weight of the formulation.

According to some embodiments, the cosmetic stabilizing system comprises arginine levulinate ranging from about 0.25 to about 1.00 wt %, inclusive, i.e., 0.25 wt %, 0.3 wt %, 0.35 wt %, 0.4 wt %, 0.45 wt %, 0.5 wt %, 0.55 wt %, 0.6 wt %, 0.65 wt %, 0.7 wt %, 0.75 wt %, 0.8 wt %, 0.85 wt %, 0.9 wt %, 0.95 wt %, or 1.00 wt % of the composition and arginine anisate ranging from about 0.05 wt % to about 0.50 wt % of the composition, inclusive, i.e., 0.05 wt %, 0.1 wt %, 0.15 wt %, 0.2 wt %, 0.25 wt %, 0.3 wt %, 0.35 wt %, 0.4 wt %, 0.45 wt %, or 0.5 wt % of the composition.

According to some embodiments, pH of the cosmetic composition stabilizing system comprising arginine, p-anisic acid, and levulinic acid ranges from about pH 4.0 to about pH 6.0, inclusive, i.e., about pH 4.0, about pH 4.1, about pH 4.2, about pH 4.3, about pH 4.4, about pH 4.5, about pH 4.6, about pH 4.7, about pH 4.8, about pH 4.9, about pH 5.0, about pH 5.1, about pH 5.2, about pH 5.3, about pH 5.4, about pH 5.5, about pH 5.6, about pH 5.7, about pH 5.8, about pH 5.9, or about pH 6.0.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will occur unless a third substance, an emulsifying agent, is incorporated.

An emulsifying agent (emulsifier) is a compound or substance that acts as a stabilizer for emulsions, preventing liquids that ordinarily don't mix from separating by increasing the kinetic stability of the mixture. The chemical structure of many of these agents have both a hydrophilic and a lipophilic part. All emulsifying agents concentrate at and are adsorbed onto the oil:water interface to provide a protective barrier around the dispersed droplets. In addition to this protective barrier, emulsifiers stabilize the emulsion by reducing the interfacial tension of the system. Some agents enhance stability by imparting a charge on the droplet surface thus reducing the physical contact between the droplets and decreasing the potential for coalescence.

Emulsifying agents can be classified according to: 1) chemical structure; or 2) mechanism of action. Classes according to chemical structure are synthetic, natural, finely dispersed solids, and auxiliary agents. Classes according to mechanism of action are monomolecular, multimolecular, and solid particle films. [https://pharmlabs.unc.edu/labs/emulsions/prep.htm, visited Oct. 16, 2020]

Exemplary synthetic emulsifying agents include cationic, e.g., benzalkonium chloride, benzethonium chloride; anionic, e.g., alkali soaps (sodium or potassium oleate); amine soaps (triethanolamine stearate); detergents (sodium lauryl sulfate, sodium dioctyl sulfosuccinate, sodium docusate); and nonionic, e.g., sorbitan esters (Spans®), polyoxyethylene derivatives of sorbitan esters (Tweens®), or glycerylesters Cationic and anionic surfactants are generally limited to use in topical, o/w emulsions. [Id.]

A variety of emulsifiers are natural products derived from plant or animal tissue. These form hydrated lyophilic colloids (called hydrocolloids) that form multimolecular layers around emulsion droplets. Hydrocolloid type emulsifiers have little or no effect on interfacial tension, but exert a protective colloid effect, reducing the potential for coalescence, by: providing a protective sheath around the droplets; imparting a charge to the dispersed droplets (so that they repel each other); and swelling to increase the viscosity of the system (so that droplets are less likely to merge). Examples of hydrocolloid emulsifiers include, without limitation, vegetable derivatives, e.g., acacia, tragacanth, agar, pectin, carrageenan, lecithin; animal derivatives, e.g., gelatin, lanolin, cholesterol; Semi-synthetic agents, e.g., methylcellulose, carboxymethylcellulose; and synthetic agents, e.g., Carbopols®. The animal derivatives general form w/o emulsions. Lecithin and cholesterol form a monomolecular layer around the emulsion droplet instead of the typically multimolecular layers. Cholesterol is a major constituent of wool alcohols; it gives lanolin the capacity to absorb water and form a w/o emulsion. Lecithin (a phospholipid derived from egg yolk) produces o/w emulsions because of its strong hydrophilic character. [Id.]

Finely divided or finely dispersed solid particle emulsifiers form a particulate layer around dispersed parties. Most will swell in the dispersion medium to increase viscosity and reduce the interaction between dispersed droplets. Most commonly they support the formation of o/w emulsions, but some may support w/o emulsions. Examples include bentonite, veegum, hectorite, magnesium hydroxide, aluminum hydroxide and magnesium trisilicate. [Id.]

The hydrophile-lipophile balance (HLB) system is used to describe the characteristics of a surfactant, one class of emulsifiers, which lower surface tension between liquids or between a solid and liquid. It is an arbitrary scale to which HLB values are experimentally determined and assigned. If the HLB value is low, the number of hydrophilic groups on the surfactant is small, which means it is more lipophilic (oil soluble) than hydrophilic (water soluble). Conversely, if the HLB value is high, there are a large number of hydrophilic groups on the surfactant, which makes it more hydrophilic (water soluble) than oil soluble. An HLB value of 10 or higher means that the agent is primarily hydrophilic.

Emulsifying agents (HLB 3-6 (w/o) and 8-18 (o/w) are surfactants that reduce the interfacial tension between oil and water, thereby minimizing the surface energy through formation of globules; examples include, glyceryl monostearate, methylcellulose, sodium lauryl sulfate, sodium oleate, sorbitan monopalmitate, sorbitan monostearate, sorbitan tristrearate, tragacanth, triethanolamine oleate, polyoxethylene sorbitan monolaurate; poloxamer (Pluronic F-68)).

According to some embodiments, the cosmetic compositions of the described invention may contain a viscosity enhancing agent or thickener. Viscosity enhancing agents are agents that thicken, gel or harden the composition. According to some embodiments, the viscosity enhancing agent is derived from botanical extracts. Exemplary viscosity enhancing agents include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and/or mixtures thereof.

According to some embodiments, the emulsifying agent is Ecogel™, a commercially available phospholipid-based gelling agent with emulsifying properties comprising lysolecithin, sclerotium gum, Xanthan gum and pullulan. [Bay House Ingredients, Milton Keynes, England]. Ecogel™ is stable over a wide pH range (pH 2.0-10.0) and can be used as 0.25-1.0% wt % of the cosmetic formulation. According to some embodiments, the cosmetic composition formulated for topical application is a slightly viscousnon-occluded water based liquid comprising a cosmetic composition stabilizing system comprising arginine, p-anisic acid, and levulinic acid. According to some embodiments, the pH of the finished product ranges from about 4.0 to about 5.0, inclusive, i.e., 4.0, 4.1, 4.2, 4.3, 4.4, 4.5, 4.6, 4.7, 4.8, 4.9, or 5.0. According to some embodiments, viscosity of the composition ranges from 5000 to 7500 centipoise, i.e., about 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, or 7500 centipoise.

Components of an exemplary formulation are shown in Table 2.

TABLE 2
Exemplary formulation pH: 4.0-5.0

| Component | Wt % |
|---|---|
| LMWHA | 0.10-0.50% |
| HMWHA | 0.5-1.50% |
| 20% active form, CBD isolate | 1.0-5.0% |
| Arginine anisate | 0.05-0.50% |
| Arginine levulinate | 0.25-1.00% |
| pH adjusting agent | 0.01-0.50% |
| Emulsifying agent | 0.25-1.00% |
| Water | 90-99% |
| Total | 100.00 |

### Methods

According to another aspect, the described invention provides a method for promoting/maintaining perianal tissue vitality and tissue health in a subject, comprising administering topically a cosmetic composition comprising a water-based gel component; a botanical ingredient component, and a cosmetic composition stabilizing system, wherein pH of the cosmetic composition ranges from 4.0 to 5.0, inclusive, i.e., 4.0, 4.1, 4.2, 4.3, 4.4, 4.5, 4.6, 4.7, 4.8, 4.9, or 5.0. According to some embodiments, the subject is a female subject. According to some embodiments, the cosmetic composition reduces one or more cutaneous symptom(s) of hemorrhoidal disease. According to some embodiments, the hemorrhoidal disease comprises external hemorrhoids. According to some embodiments, cutaneous symptoms of hemorrhoidal disease include, without limitation, one or more of pain or itching. According to some embodiments, the cosmetic composition may have one or more of the following cosmetic effects: reduce symptoms and signs of trauma, insult or injury (e.g., dryness, burning, irritation, discomfort or pain); improving healing and rejuvenation of the wounded tissue (e.g., improve tissue strength) According to some embodiments, the cosmetic composition reduces pain, itching or both of external hemorrhoid tissue. According to some such embodiments, the cosmetic composition promotes wound healing of inflamed external hemorrhoidal tissue. According to some embodiments, the described soothing composition may restore characteristics of a healthy tissue through healing, rejuvenation, or both.

According to another aspect, the described invention provides a method for promoting/maintaining vaginovulval tissue vitality and tissue health in a female subject, comprising administering topically a cosmetic composition comprising a water-based gel component; a botanical ingredient component, and a cosmetic composition stabilizing system, wherein pH of the cosmetic composition ranges from 4.0 to 5.0, inclusive, i.e., 4.0, 4.1, 4.2, 4.3, 4.4, 4.5, 4.6, 4.7, 4.8, 4.9, or 5.0.

According to some embodiments, the female subject is a subject susceptible to or experiencing genitourinary symptoms of trauma, insult or injury. According to some embodiments, the female subject is a menopausal subject. According to some embodiments, the female subject is a diabetic subject. According to some embodiments, the female subject is a subject that will undergo, is undergoing or has undergone treatment comprising radiation therapy to treat a gynecologic cancer, e.g., endometrial cancer, cervical cancer; ovarian cancer; vulvar cancer. According to some embodiments, the female subject is a breast cancer survivor.

According to some embodiments, parameters of vaginovulval tissue vitality include, without limitation, improved tissue strength, appropriate vaginal pH; reduced susceptibility to trauma/mechanical insult, reduced inflammation, reduced itching, and improved tissue elasticity.

According to some embodiments, the cosmetic composition may have one or more of the following cosmetic effects: reduce symptoms and signs of trauma, insult or injury (e.g., dryness, burning, irritation, discomfort or pain); improving healing and rejuvenation of the wounded tissue (e.g., improve tissue strength) reducing clinical signs of dryness and insufficient hydration (e.g., loss of elasticity, inflammation), or modulation of vaginal pH, inflammation, or itching. According to some embodiments, the cosmetic condition may create an environment that facilitates healing and rejuvenation of wounded tissue. According to some embodiments, the described soothing composition may restore characteristics of a healthy tissue through healing, rejuvenation, or both.

### Water-Based Gel Component

### Background

Hyaluronic acid (HA) or hyaluronan is a member of the large family of glycosaminoglycans (GAGs), which are the main components of the extracellular matrix. The HA molecule is composed of a repeating unit of D-glucuronic acid and N-acetyl-D-glucosamine bound with β-glycosidic linkages.

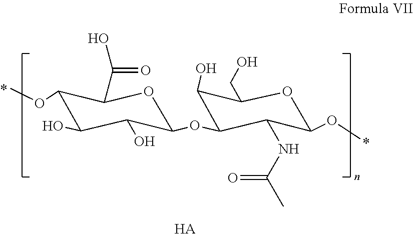

This simple molecular unit forms a long linear polymer, with molecular weight reaching 5×10⁶ kDa. Long hyaluronan polymers have the ability to bind large amounts of water.

In its native form as a very long polymer, hyaluronan is known as high molecular weight (HMW) hyaluronan. In certain conditions, it can be decomposed into small fragments referred to as low molecular weight HA. [Litwiniuk, M. et al. Wounds (2016) 28 (3): 78-88, citing Aya, K L & Stern, R. Wound Repair Regen. (2014) 22(5): 579-93]. In somatic tissues, hyaluronidase-1 (Hyal-1) and hyaluronidase-2 (Hyal-2) are responsible for HA degradation. First, Hyal-2, a cell-membrane liked enzyme, degrades HA to fragments with molecular eight reaching 20 kDa. These HA molecules are subsequently endocytosed and delivered to lysosomes, where further digestion is performed by Hyal-1. [Id., citing Stern, R. & Jedrzejas, M. Chem. Rev. (2006) 106 (30: 818-39). In injured tissue, free radicals also can decompose HA polymers into smaller fragments. [Id., citing Longacre, M T et al. Ann. Surg. (1991) 213 (4): 292-96].

It has been well documented that HMWHA displays anti-inflammatory and immunosuppressive properties, whereas LMWHA degradation products of HA can induce inflammation. [Id., citing Prevo, R. et al. J. Biol. Chem. (2001) 276 (22): 19420-30]. Small hyaluronan fragments have been shown to increase the expression and protein production of several cytokines, such as MMP-12, plasminogen activator inhibitor-1 [Id., citing Horton, M R et al. Am. J. Physiol. Lung Cell Mol. Physiol. (2000) 279 (4): L707-15; Horton, M R, et a. J. Immunol. (1999) 162 (7): 4171-76], macrophage inflammatory protein-1a (MIP-1a), monocyte chemoattractant 1, keratinocyte chemoattractant, interleukin-8 (IL-8) and IL-12 by macrophages. [Id., citing Horton, M R, et al. J. Immunol. (1998) 160 (6): 3023-30; Hodge-DuFour, J. et al. J. Immunol. (1997) 159 (5): 2492-2500; McKee, C M et al. J. Clin. Invest. (1996) 98 (10): 1403-13]. While CD44 is the main receptor for hyaluronan, other receptors, such as TLRs also are involved in HA signaling. [Id., citing Teder, P. et al. Science (2002) 296 (5565): 155-58]. LMWHA is able to bind to TLR receptors and initiate the signaling cascade leading to the production of pro-inflammatory cytokines and chemokines in various types of cells in vitro. [Id., citing Litwiniuk, M. et al. Cent. Eur. J. Immunol. (2009) 34 (4): 247-51]. In immune cells from injured tissues, TLR2 and TLR4 activation by LMWHA has been shown to lead to initiation of MyD88-dependent NFκB signaling cascade and pro-inflammatory cytokine gene expression. [Id., citing Jiang, D. et al. Nat. Med. (2005) 11 (11): 1173-79; Suga, H. et al. J. Dermatol. Sci. (2014) 73 (2): 117-24]. Induction by LMWHA TLR-related myeloid differentiation primary response gene 88 (MyD88)/NFκB signaling also was confirmed in breast tumor cells. Small HA fragments were shown to stimulate CD44 association with TLR2, TLR4 and MyD88, leading to NF-κB-specific transcriptional activation and the expression of proinflammatory cytokines Il-1β and IL-8 in a human breast cell line. Taken together, these reports suggest that LMWHA induces inflammation via activation of TLR receptors and initiation of MyD8/NFκB signaling, which leads to production of proinflammatory cytokines and chemokines.

In physiological conditions, the activation of immune system cells is crucial for proper wound healing. In acute wounds, small hyaluronan fragments accumulating at the site of injury activate the immune system to manage rupture in tissue integrity; however, in chronic wounds, a constant excessive inflammatory response proves to actually prevent that wound from healing. [Id.]

LMWHA, which has strong antioxidant properties, has shown protective effects against ROS both in vitro and in vivo, and inhibits lipid peroxidation and scavenges free radicals. [Id., citing Ke, C. et al. Food Chem. Toxicol. (2011) 49 (10): 2670-75].

The anti-inflammatory potential of HMWHA has been well documented in osteoarthritis. Intra-articular injection of HMWHA has been used to treat osteoarthritis, since HA is a basic component of normal synovial fluid and the concentration of HA is decreased in osteoarthritis affected joints. For example, HMWHA was able to inhibit IL-1β expression in synoviocytes in a rabbit model of osteoarthritis. [Id., citing Miki, Y. et al. Inflamm. Res. (2010) 59 (6): 471-77]. IL-1 dependent expression of MMP-1 and MMP-3 was reduced in human synoviocytes by HMWHA treatment. In a large study, the influence of HMWHA on gene expression of various inflammatory cytokines by human fibroblast-like synoviocytes (FLS) in patients with early-stage osteoarthritis was analyzed. [Id., citing Wang, C T, et al. osteoarthritis Cartilage (2006) 14 (12): 1237-47] They reported the downregulation of IL-8 and iNOS gene expression in unstimulated FLS and aggrecanase-2, and tumor necrosis factor alpha (TNFα) gene expression in IL-1 stimulated FLS. Blocking the CD44 receptor with anti-CD44 antibody inhibited the down-regulatory effects of HMWHA on gene expression. [Id., citing Bourguignon, L Y et al. Cytoskeleton (Hoboken) (2011) 68 (12): 671-93].

The exact mechanism in which HMWHA interacts with TLR receptors, leading to inhibition of inflammatory cascades is not known. HWHA was able to significantly diminish TLR4, TLR2, MyD88 and NF-κB expression in synoviocytes in a murine model of osteoarthritis. [Id., citing Campo, G M et al. Biochim. Biophys. Acta (2011) 1812 (9): 1170-81]. They also observed reduced mRNA expression and protein production for TNFα, IL-1β, IL-17, MMP-13 and inducible nitrous oxide synthase gene in arthritic mice treated with HMWHA [Id., citing Campo, G M et al. Biochim. Biophys. Acta (2011) 1812 (9): 1170-81], but only when HWHA was administered in an early inflammatory phase of osteoarthritis.

Examples of antioxidant effects of HMWHA include a decrease in ultraviolet B-induced apoptosis and EDTA-induced oxidative damage of DNA [Id., citing Bourguignon, L Y et al. J. Biol Chem. (1997) 272 (44): 27913-27918; Pauloin, T. et al., Mol. Vis. (2009) 15: 577-83], and a decrease in apoptosis and oxidative stress triggered with benzalkonium chloride and sodium lauryl sulfate detergents which are widely used in ophthalmic preparations [Id., citing Pauloin, T. et al. Cornea (2009) 28 (9): 1032-41; Pauloin, T. et al. Eur. J. Pharm. Sci. (20008) 34 (4-5): 263-73]. The mechanisms by which HMWHA reduces oxidative stress are still not understood.

Numerous studies have shown that HA signaling plays a role in angiogenesis regulation, mainly by influencing endothelial cell behavior. Both HMWHA and LMWHA are potent regulators of angiogenesis. LMWHA stimulates vascular EC proliferation, migration, and tubule formation in vitro, as well as in various in vivo models of angiogenesis, while HMWHA displays antiangiogenic properties by inhibiting EC proliferation motility and sprout formation. [Id., citing Toole, B P. Nat. Rev. Cancer (2004): 4(7): 528-39]. The exact molecular mechanism determining the proangiogenic or antiangiogenic effects of different HA forms have not been fully elucidated. A context-dependent response to different forms of HA and a possible role of the microenvironment in this process has been suggested. In wound closure assays, adding CXCL12 to the culture medium significantly increased cell migration and induced faster wound closure. This effect was statistically augmented when cells were preincubated with HMWHA. [Id., citing Fuchs, K. et al. Cell Death Dis. (2013) 4: e819]. In vitro studies have shown that CXCR4 activation by CXCL12 was significantly increased in HUVECs pretreated with HMWHA, whereas preincubation with LMWHA blocked CXCL12 signaling in these cells.

According to some embodiments, a sodium hyaluronate product (e.g., HMWHA, LMWHA, or both) is produced by biofermentation in the strain Steptococcus equi sub. zooepidemicus. According to some embodiments, the sodium hyaluronate product(s) is (are) obtained from a commercial source (e.g., Bloomage Biotechnology Corp., Ltd., Jinan, China). According to some embodiments, the main components of the biofermentation medium include: wheat peptone, yeast extract powder, and glucose.

According to some embodiments, the molecular weight of the HMWHA ranges from 1,000-1,800 kDa, inclusive, i.e., at least 1000, 1100, 1200, 1300, 1400, 1500, 1600, 1700, or 1800 kDa. According to some embodiments, the molecular weight of the LMWHA is less than 10 kDa, i.e., at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, at least 9 kDa to less than 10 kDa.

According to some embodiments, the finished product comprises from about 0.10 to about 0.50 wt %, inclusive of the LMWHA, i.e., 0.10 wt %, 0.15 wt %. 0.20 wt %, 0.25 wt %, 0.30 wt %, 0.35 wt %, 0.40 wt %, 0.45 wt %, or 0.50 wt % LMWHA, and from about 0.50 to about 1.50 wt % HMWHA, inclusive, i.e., 0.50 wt %, 0.60 wt %, 0.70 wt %, 0.80 wt %, 0.90 wt %, 1.0 wt %, 1.2 wt %, 1.2 wt %, 1.3 wt %, 1.4 wt %, or 1.5 wt % HMHWA.

According to some embodiments the ratio of HMWHA to LMWHA ranges from 1:0.7 to 1:1, inclusive, i.e., 1:0.7, 0.8, 0.9 or 1.0.

### Botanical Ingredient Component

According to some embodiments, the botanical ingredient component is a cannabinoid. According to some embodiments, the cannabinoid is derived from hemp. According to some embodiments, the cannabinoid is not a full spectrum CBD, i.e., it does not contain all the cannabinoids found in the cannabis plant in nature. According to some embodiments, the cannabinoid is nonpsychoactive. According to some embodiments, the cannabinoid is a decarboxylated CBD.

According to some embodiments, the isolate comprises about 20% decarboxylated CBD. According to some embodiments, the isolate comprising about 20% decarboxylated CBD is in the form of a THC free nano-infused water soluble powder. According to some embodiments, the isolate comprising about 20% decarboxylated CBD is obtained from a commercial source (e.g., Global Cannabinoids, Las Vegas, Nev.). According to some embodiments, the isolate comprising about 20% CBD comprises <1% cannabidivarin (CBDV), which has a similar molecular structure to CBD, but instead of having a pentyl chain, it has a propyl chain. According to some embodiments, decarboxylation is by heat not to exceed 300° F., e.g., 200° F. (93° C.) for 75 min, 225° F. (107° C.) for 50 min, or 250° F. (121° C.) for 30 min.

An exemplary analysis of a THC-free nano-infused water soluble cannabinoid powder is shown below (LOQ=limit of quantitation). The reported result is based on a sample weight with the applicable moisture content for that sample; unless otherwise stated all quality control samples performed within specifications established by the Laboratory.

| Analyte | LOQ % | Mass % | Mass mg/g |
|---|---|---|---|
| THCa | 0.010 | <LOQ | <LOQ |
| Δ9-THC | 0.010 | <LOQ | <LOQ |
| CBDa | 9.010 | <LOQ | <LOQ |
| CBD | 0.102 | 18.218 | 18.218 |
| CBC | 0.005 | <LOQ | <LOQ |
| CBG | 0.005 | <LOQ | <LOQ |
| CBN | 0.010 | <LOQ | <LOQ |
| THCV | 0.005 | <LOQ | <LOQ |
| Δ8-THC | 0.005 | <LOQ | <LOQ |
| CBGa | 0.005 | <LOQ | <LOQ |
| CBDV | 0.005 | 0.051 | 0.051 |
| Total | | 18.269 | 18.269 |

According to some embodiments, total CBD can be represented as the formula CBDa*0.877+CBD, and total THC can be represented as THCa*0.877+Δ9-THC+Δ8THC. According to some embodiments, the powder contains one or more inactive ingredients, e.g., tapioca maltdextrin.

According to some embodiments, the finished product comprises from 1.0 wt % to about 5.0 wt %, inclusive of the 20% CBD-THC-free nanoinfused water-soluble powder, i.e., at least 1.0 wt %, at least 1.25 wt %, at least 1.5 wt %, at least 1.75 wt %, at least 2.0 wt %, at least 2.25 wt %, at least 2.5 wt %, at least 2.75 wt %, at least 3.0 wt %, at least 3.25 wt %, at least 3.5 wt %, at least 3.75 wt %, at least 4.0 wt %, at least 4.25 wt %, at least 4.5 wt %, at least 4.75 wt %, or at least 5 wt %.

### Cosmetic Composition Stabilizing System

According to some embodiments, the cosmetic composition stabilizing system comprises an effective amount of an arginine component. According to some embodiments, the arginine component comprises i) arginine, or a conjugate, or an analog thereof, ii) an organic acid, a conjugate, or an analog thereof, and iii) a solvent. As used herein, the phrase “arginine, a conjugate, or an analog thereof” is referred to as an “arginine compound.” As used herein, the phrase “organic acid a conjugate, or a derivative thereof” is referred to as an “organic acid compound.” According to some embodiments the arginine component comprises i) an arginine compound, ii) an organic acid compound, and iii) a solvent.

According to some embodiments, the cosmetic composition stabilizing system has a preservative effect on the composition, a solubilizing effect, a stabilizing, a neutralizing, or anti-microbial effect on the composition, a moisturizing, and/or healing effect, or any combination thereof. For example, according to some embodiments the cosmetic composition stabilizing system has a preservative effect on the composition and/or a solubilizing effect on the active agent. According to some embodiments the cosmetic composition stabilizing system has a preservative effect on the composition, and/or a solubilizing effect on the active agent, and/or a stabilizing effect on the composition. According to some embodiments, the cosmetic composition stabilizing system has a preservative effect on the composition, and/or a solubilizing effect on the active agent, and/or a stabilizing effect and/or a neutralizing effect on the composition. According to some embodiments, the cosmetic composition stabilizing system has a preservative effect on the composition, and/or a solubilizing effect on the active, and/or a stabilizing, neutralizing, and/or anti-microbial effect on the composition. According to some embodiments, the cosmetic composition stabilizing system has a preservative effect on the composition, and/or a solubilizing effect on the active, a stabilizing, and/or a neutralizing, and/or an anti-microbial effect on the composition. According to some embodiments, the cosmetic composition stabilizing system has a preservative effect on the composition, a solubilizing effect on the active, and/or a stabilizing, a neutralizing, and/or an anti-microbial effect on the composition and/or a moisturizing cosmetic/therapeutic effect. According to some embodiments, the cosmetic composition stabilizing system has a preservative effect on the composition, and/or a solubilizing effect on the active, a stabilizing, neutralizing, and/or anti-microbial effect on the composition, and/or a moisturizing cosmetic/therapeutic effect. According to some embodiments, the cosmetic composition stabilizing system has a preservative effect on the composition, and/or a solubilizing effect on the active, and/or a stabilizing, a neutralizing, and/or an anti-microbial effect on the composition, and/or a moisturizing cosmetic/therapeutic effect. According to some embodiments, the cosmetic composition stabilizing system has a preservative effect on the composition, and/or a solubilizing, stabilizing, neutralizing, and/or anti-microbial effect on the composition, and/or a moisturizing, exfoliating and/or healing cosmetic/therapeutic effect.

According to some embodiments, the arginine compound is arginine, or a salt, conjugate, or analog thereof. For example, the arginine may be D,L-arginine, D-arginine, L-arginine, alkyl (ethyl, methyl, propyl, isopropyl, butyl, isobutyl, t-butyl) esters of arginine and conjugates and analogs thereof.

According to some embodiments, the arginine conjugate may be a conjugate acid or a conjugate base. For example, the arginine conjugate may be argininum, or argininate.

According to some embodiments, the arginine compound may be employed in any suitable amount. For example, the arginine compound may be present in the arginine component in at least about 1.0 wt %, at least about 5.0 wt %, at least 10.0 wt %, at least about 15.0 wt %, at least 20.0 wt %, at least 25.0 wt %, at least 30.0 wt %, at least 35.0 wt %, at least 40.0 wt %, at least 45.0 wt %, at least 50.0 wt %, at least 55.0 wt %, at least 60.0 wt %, at least 65.0 wt %, at least 70.0 wt %, at least 75.0 wt %, at least 80.0 wt %, at least 85.0 wt %, at least 90.0 wt %, and about 95.0 wt % of the total weight of the arginine component. Exemplary amounts include 10.0 wt % to about 70.0 wt %, inclusive, based upon the total weight of the arginine component, about 20 wt % to about 60 wt % inclusive, based upon the total weight of the arginine component, about 30 wt % to about 50 wt % inclusive of the total weight of the arginine component.

According to some embodiments, the organic acid compound can be an organic acid, its conjugate, or an analog thereof. According to some embodiments the organic acid can be any suitable organic acid. For example, organic acids may be substituted and non-substituted aliphatic (saturated and unsaturated) and aromatic acids. Organic acids may possess as substituents one or more functional groups, such as alkyl, alkenyl, alkynyl, halogen, hydroxy, carbonyl, carboxylic acid, aldehyde, ester, amide, carbonate, carbamate, ether, amino, cyano, isocyano, oxy, oxo, thia, aza, azide, imine, nitro, nitrate, nitroso, nitrosooxy, cyanate, isocyanate, thiocyanate, isothiocyanate, sulfinyl, sulfhydryl, sulfonyl, phosphino, wherein each of the alkyl, alkenyl, alkynyl and amino groups may themselves be optionally substituted with one or more of the preceding functional groups. According to some embodiments, some functional groups, such as hydroxy, will impart or augment a character to the acid that is suitable for the present composition, such as a hygroscopic character. According to some embodiments, the organic acid contains multiple carboxylic acid groups.

According to some embodiments, the organic acid is a hydroxy acid. According to some embodiments, the hydroxy acid is an α-hydroxy acid (AHA), a β-hydroxy acid (BHA), a γ-Hydroxy acids (GBA), an ω-Hydroxy acids, a monohydroxy acid (MHA), a polyhydroxy acid (PHA)/polycarboxy hydroxy acid (PCHA), an aliphatic hydroxy acid (AlHA), an aromatic hydroxy acids acid (ArHAs), an arylaliphatic hydroxy acid (AAHA), a hydroxy fatty acids, and the like.

According to some embodiments, the hydroxy acid is an α-hydroxy acid (AHA). The alpha (α) carbon in organic molecules refers to the first carbon atom that attaches to a functional group, such as a carbonyl. According to some embodiments, the AHA is an alkyl AHA, arylalkyl AHA, or a polycarboxyl AHA.

According to some embodiments, the organic acid component comprises anisic acid, also known as 2-methoxybenzoic acid; o-Anisic acid; 579-75-9; o-Methoxybenzoic acid; 2-Anisic acid; O-Methylsalicylic acid; Benzoic acid, 2-methoxy-;Salicylic acid methyl ether; 2-Methoxy-benzoic acid; 529-75-9, molecular formula C₈H₈O₃.

According to some embodiments, the organic acid component comprises levulinic acid also known as 4-Oxopentanoic acid; 123-76-2; Laevulinic acid; Pentanoic acid, 4-oxo-4-Oxovaleric acid; Levulic acid; 3-Acetylpropionic acid; 4-Ketovaleric acid; LEVA; molecular formula C₅H₈O₃.

According to some embodiments, the organic acid component comprises mandelic acid, also known as 17199-29-0; (S)-(+)-Mandelic acid; (S)-Mandelic acid; (S)-2-Hydroxy-2-phenylacetic acid; L-mandelic acid; L-(+)-mandelic acid; S-(+)-Mandelic acid; (2S)-2-hydroxy phenylacetic acid; UNII-L0UMW58G3T; l(+)-mandelic acid; molecular formula C₈H₈O₃.

According to some embodiments, the organic acid component comprises salicylic acid, also known as 2-Hydroxybenzoic acid; 69-72-7; o-hydroxybenzoic acid; 2-Carboxyphenol; o-Carboxyphenol; Rutranex; Salonil; Retarder W; Keralyt, molecular formula C₇H₆O₃ or HOC₆H₄COOH.

According to some embodiments, the organic acid component comprises sorbic acid, also known as 110-44-1; 2,4-Hexadienoic acid; (2E,4E)-hexa-2,4-dienoic acid; 2E,4E-Hexadienoic acid; Panosorb; Sorbistat; 2-Propenylacrylic acid; trans, trans-Sorbic acid; Hexadienoic acid, molecular formula C₆H₈O₂ or CH₃CH═CHCH═CHCOOH.

According to some embodiments, the organic acid component comprises benzoic acid, also known as 65-85-0; Dracylic acid; benzenecarboxylic acid; Carboxybenzene; Benzeneformic acid; phenylformic acid; Benzenemethanoic acid; Phenylcarboxylic acid; Retardex, molecular formula C₇H₆O₂ or C₆H₅COOH.

According to some embodiments, the organic acid component comprises ferulic acid, also known as trans-4-Hydroxy-3-methoxycinnamic acid, and trans-Ferulic acid, molecular formula HOC₆H₃(OCH₃)CH═CHCO₂H.

According to some embodiments, the organic acid component comprises syringic acid, also known as 3,5-Dimethoxy-4-hydroxybenzoic acid, 4-Hydroxy-3,5-dimethoxy-benzoic acid, and Gallic acid 3,5-dimethyl ether, molecular formula HOC6H₂(OCH₃)₂CO₂H.

According to some embodiments, the organic acid component comprises two organic acids selected from the group consisting of anisic acid, levulinic acid; mandelic acid; salicylic acid, sorbic acid, benzoic acid, ferulic acid, and syringic acid, e.g. anisic acid and levulinic acid; anisic acid and mandelic acid; anisic acid and salicylic acid, anisic acid and sorbic acid, anisic acid and benzoic acid, anisic acid and ferulic acid, anisic acid and syringic acid; levulinic acid and mandelic acid, levulinic acid and salicylic acid, levulinic acid and sorbic acid, levulinic acid and benzoic acid, levulinic acid and ferulic acid, levulinic acid and syringic acid; mandelic acid and salicylic acid, mandelic acid and sorbic acid, mandelic acid and benzoic acid, mandelic acid and ferulic acid, mandelic acid and syringic acid; saliculic acid and sorbic acid, salicylic acid and benzoic acid, salicylic acid and ferulic acid, salicylic acid and syringic acid, sorbic acid and benzoic acid, sorbic acid and ferulic acid, sorbic acid and syringic acid; benzoic acid and ferulic acid, benzoic acid and syringic acid; ferulic acid and syringic acid.

According to some embodiments, the organic acids, when used as a combination of two as exemplified above (for example, levulinic acid and anisic acid, etc.), together with glyceryl caprylate/caprate form a broad spectrum preservative effective against microbial contaminants, e.g., bacteria, yeast and mold. Without being limited to any particular theory, the stabilization system comprising glyceryl caprylate/caprate allows for greater penetration of both acids into the cell wall of a targeted organism, which means that the overall concentration of the acids in the formulation is lower, and the concentration of free acid required for full spectrum preservation of the product is reduced. This in turn increases efficacy in challenging finished formulations at a physiological pH, and reduces the need for additional ingredients in the finished formulation without extreme adjustments of the final pH of the finished formulation.

According to some embodiments, the organic acid compound includes any derivative of an organic acid thereof. For example, a derivative of an organic acid that would revert to their acid form when contacted with water, includes, without limitation, an easily hydrolyzable anhydride, mixed anhydride and ester derivatives of the organic acids.

According to some embodiments, the organic acid compound may be employed in any suitable amount. For example, the organic acid compound may be present in the arginine component as about 1.0 wt %, 5.0 wt %, 10.0 wt %, 15.0 wt %, 20.0 wt %, 25.0 wt %, 30.0 wt %, 35.0 wt %, 40.0 wt %, 45.0 wt %, 50.0 wt %, 55.0 wt %, 60.0 wt %, 65.0 wt %, 70.0 wt %, 75.0 wt %, 80.0 wt %, 85.0 wt %, 90.0 wt %, and about 95.0 wt % of the total weight of the arginine component. Exemplary amounts include about 10.0 wt %, to about 70.0 wt % based upon the total weight of the arginine component, about 20 wt % to about 60 wt % based upon the total weight of the arginine component, about 30 wt % to about 50 wt % of the total weight of the arginine component.

According to some such embodiments, the pH is high enough to keep the acid in solution and low enough to keep the glyceryl caprylate/caprate from hydrolyzing. For example, the pH of the raw material cosmetic composition stabilization system in water comprising glyceryl caprylate/caprate can range from pH 4.1-6.9, inclusive, i.e., pH 4.1, 4.2, 4.3, 4.4, 4.5, 4.6, 4.7, 4.8, 4.9, 5.0, 5.1, 5.2, 5.3, 5.43, 5.5, 5.6, 5.7, 5.8, 5.9, 6.0, 6.1, 6.2, 6.3, 6.4, 6.5, 6.6, 6.7, 6.8, 6.9. According to some embodiments, the pH of the finished product/cosmetic composition comprising glyceryl caprylate/caprate ranges from pH 6.0-6.5, inclusive, i.e., pH 6.0, 6.1, 6.2, 6.3, 6.4, 6.5.

According to some embodiments, the arginine component of the cosmetic composition stabilizing system comprises a solvent. Examples of the solvent may include water, low molecular weight alcohols such as C₁₋₆ branched or straight chain alcohols, e.g., methanol, ethanol and isopropanol, low molecular weight ketones such as C₁₋₆ branched or straight chain ketones, e.g., acetone, aromatic compounds and low molecular weight alkanes, such as C₁₋₁₀ branched or straight chain alkanes.

According to some embodiments, the arginine component comprises a polar solvent. Exemplary polar solvents include: water; alcohols (such as ethanol, propyl alcohol, isopropyl alcohol, hexanol, benzyl alcohol, polyhydric alcohol); polyols (such as propylene glycol, polypropylene glycol, butylene glycol, hexylene glycol, polyethylene glycols), sugar alcohols (e.g., malitol, sorbitol), glycerine; panthenol dissolved in glycerine, flavor oils and mixtures thereof. Mixtures of these solvents can also be used. According to some embodiments, the solvent is water.

According to some embodiments, the solvent may be employed in any suitable amount. For example, the solvent may be present in the arginine component in about 1.0 wt %, 5.0 wt %, 10.0 wt %, 15.0 wt %, 20.0 wt %, 25.0 wt %, 30.0 wt %, 35.0 wt %, 40.0 wt %, 45.0 wt %, 50.0 wt %, 55.0 wt %, 60.0 wt %, 65.0 wt %, 70.0 wt %, 75.0 wt %, 80.0 wt %, 85.0 wt %, 90.0 wt %, and about 95.0 wt % of the total weight of the arginine component. Exemplary amounts include 10.0 wt %, to about 70.0 wt %, inclusive, based upon the total weight of the arginine component, about 20.0 wt % to about 60.0 wt %, inclusive, based upon the total weight of the arginine component, about 30.0 wt % to about 50.0 wt %, inclusive, of the total weight of the arginine component.

According to some embodiments, the range of pH of the cosmetic or dermatologic formulation stabilizing system, which is within the range of pH 4.1-8.5, inclusive, i.e., pH 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, depends on the organic acids used.

According to some embodiments, the cosmetic composition stabilizing system comprising the arginine component of the present invention may be present as at least about 0.001 wt %, at least 0.005 wt %, at least 0.01 wt %, at least 0.05 wt %, at least 0.10 wt %, at least 0.50 wt %, at least 1 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt % of the total weight of the formulation.

According to some embodiments, the cosmetic composition stabilizing system comprising the arginine component of the present invention may be present in about 1.0 wt % to about 100.0 wt %, about 10.0 wt % to 70.0 wt %, or about 20.0 wt % to 50.0 wt % based on the total weight of the formulation. According to some embodiments, the arginine component of the present invention may be present in about 0.001 wt % to 10.0 wt %, about 0.1% to 5.0 wt %, or about 1.0 wt % to 3.0 wt % based on the total weight of the formulation.

According to some embodiments, the cosmetic stabilizing system comprises arginine levulinate ranging from about 0.25 to about 1.00 wt %, inclusive, i.e., 0.25 wt %, 0.3 wt %, 0.35 wt %, 0.4 wt %, 0.45 wt %, 0.5 wt %, 0.55 wt %, 0.6 wt %, 0.65 wt %, 0.7 wt %, 0.75 wt %, 0.8 wt %, 0.85 wt %, 0.9 wt %, 0.95 wt %, or 1.00 wt % of the composition and arginine anisate ranging from about 0.05 wt % to about 0.50 wt % of the composition, inclusive, i.e., 0.05 wt %, 0.1 wt %, 0.15 wt %, 0.2 wt %, 0.25 wt %, 0.3 wt %, 0.35 wt %, 0.4 wt %, 0.45 wt %, or 0.5 wt % of the composition.

According to some embodiments, pH of the cosmetic composition stabilizing system comprising arginine, p-anisic acid, and levulinic acid ranges from pH 7.0 to pH 7.9, inclusive, i.e., pH 7.0 pH 7.1, pH 7.2, pH 7.3, pH 7.4, pH 7.5, pH 7.6, pH 7.7, pH 7.8, or pH 7.9.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will occur unless a third substance, an emulsifying agent, is incorporated.

An emulsifying agent (emulsifier) is a compound or substance that acts as a stabilizer for emulsions, preventing liquids that ordinarily don't mix from separating by increasing the kinetic stability of the mixture. The chemical structure of many of these agents have both a hydrophilic and a lipophilic part. All emulsifying agents concentrate at and are adsorbed onto the oil:water interface to provide a protective barrier around the dispersed droplets. In addition to this protective barrier, emulsifiers stabilize the emulsion by reducing the interfacial tension of the system. Some agents enhance stability by imparting a charge on the droplet surface thus reducing the physical contact between the droplets and decreasing the potential for coalescence.

Emulsifying agents can be classified according to: 1) chemical structure; or 2) mechanism of action. Classes according to chemical structure are synthetic, natural, finely dispersed solids, and auxiliary agents. Classes according to mechanism of action are monomolecular, multimolecular, and solid particle films. [https://pharmlabs.unc.edu/labs/emulsions/prep.htm, visited Oct. 16, 2020]

Exemplary synthetic emulsifying agents include cationic, e.g., benzalkonium chloride, benzethonium chloride; anionic, e.g., alkali soaps (sodium or potassium oleate); amine soaps (triethanolamine stearate); detergents (sodium lauryl sulfate, sodium dioctyl sulfosuccinate, sodium docusate); and nonionic, e.g., sorbitan esters (Spans®), polyoxyethylene derivatives of sorbitan esters (Tweens®), or glycerylesters Cationic and anionic surfactants are generally limited to use in topical, o/w emulsions. [Id.]

A variety of emulsifiers are natural products derived from plant or animal tissue. These form hydrated lyophilic colloids (called hydrocolloids) that form multimolecular layers around emulsion droplets. Hydrocolloid type emulsifiers have little or no effect on interfacial tension, but exert a protective colloid effect, reducing the potential for coalescence, by: providing a protective sheath around the droplets; imparting a charge to the dispersed droplets (so that they repel each other); and swelling to increase the viscosity of the system (so that droplets are less likely to merge). Examples of hydrocolloid emulsifiers include, without limitation, vegetable derivatives, e.g., acacia, tragacanth, agar, pectin, carrageenan, lecithin; animal derivatives, e.g., gelatin, lanolin, cholesterol; Semi-synthetic agents, e.g., methylcellulose, carboxymethylcellulose; and synthetic agents, e.g., Carbopols®. The animal derivatives general form w/o emulsions. Lecithin and cholesterol form a monomolecular layer around the emulsion droplet instead of the typically multimolecular layers. Cholesterol is a major constituent of wool alcohols; it gives lanolin the capacity to absorb water and form a w/o emulsion. Lecithin (a phospholipid derived from egg yolk) produces o/w emulsions because of its strong hydrophilic character. [Id.]

Finely divided or finely dispersed solid particle emulsifiers form a particulate layer around dispersed parties. Most will swell in the dispersion medium to increase viscosity and reduce the interaction between dispersed droplets. Most commonly they support the formation of o/w emulsions, but some may support w/o emulsions. Examples include bentonite, veegum, hectorite, magnesium hydroxide, aluminum hydroxide and magnesium trisilicate. [Id.]

The hydrophile-lipophile balance (HLB) system is used to describe the characteristics of a surfactant, one class of emulsifiers, which lower surface tension between liquids or between a solid and liquid. It is an arbitrary scale to which HLB values are experimentally determined and assigned. If the HLB value is low, the number of hydrophilic groups on the surfactant is small, which means it is more lipophilic (oil soluble) than hydrophilic (water soluble). Conversely, if the HLB value is high, there are a large number of hydrophilic groups on the surfactant, which makes it more hydrophilic (water soluble) than oil soluble. An HLB value of 10 or higher means that the agent is primarily hydrophilic.

Emulsifying agents (HLB 3-6 (w/o) and 8-18 (o/w) are surfactants that reduce the interfacial tension between oil and water, thereby minimizing the surface energy through formation of globules; examples include, glyceryl monostearate, methylcellulose, sodium lauryl sulfate, sodium oleate, sorbitan monopalmitate, sorbitan monostearate, sorbitan tristrearate, tragacanth, triethanolamine oleate, polyoxethylene sorbitan monolaurate; poloxamer (Pluronic F-68)).

According to some embodiments, the cosmetic compositions of the described invention may contain a viscosity enhancing agent or thickener. Viscosity enhancing agents are agents that thicken, gel or harden the composition. According to some embodiments, the viscosity enhancing agent is derived from botanical extracts. Exemplary viscosity enhancing agents include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and/or mixtures thereof.

According to some embodiments, the emulsifying agent is Ecogel™, a commercially available phospholipid-based gelling agent with emulsifying properties comprising lysolecithin, sclerotium gum, Xanthan gum and pullulan. [Bay House Ingredients, Milton Keynes, England]. Ecogel™ is stable over a wide pH range (pH 2.0-10.0 an can be used as 0.25-1.0% wt % of the cosmetic formulation.

According to some embodiments, the cosmetic composition formulated for topical application and comprising a water-based gel component comprising LMWHA and HMWHA and a botanical ingredient component comprising decarboxylated CBD present at a concentration of about 20% in form of a THC free nano-infused water soluble powder is a slightly viscous non-occluded water based liquid comprising a cosmetic composition stabilizing system comprising arginine, p-anisic acid, levulinic acid, and lactic acid. According to some embodiments, the pH of the finished product is about 4.5. According to some embodiments, viscosity is about 5000 to 7500 centipoise, i.e., about 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, or 7500 centipoise.

Components of an exemplary formulation are shown in Table 3.

TABLE 3
Exemplary formulation pH: 4.0-5.0

| Component | wt % |
|---|---|
| LMWHA | 0.10-0.50% |
| HMWHA | 0.50-1.50% |
| 20% active form, CBD isolate | 1.0-5.0% |
| Arginine anisate | 0.05-0.50% |
| Arginine levulinate | 0.25-1.00% |
| pH adjusting agent | 0.01-0.50% |
| Stabilizing agent | 0.25-1.00% |
| Water | 90-99% |
| Total | 100.00 |

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Introduction

The term “skin” as used herein refers to the membranous protective covering of the body consisting of the epidermis and corium (dermis).

### Anatomy and Physiology of the Skin

The skin is the largest organ in the body consisting of several layers and plays an important role in biologic homeostasis. Its reapproximation over the surface of the wound has long been a primary sign of the completion of a significant portion of wound healing. This reclosure of the defect restores the protective function of the skin, which includes protection from bacteria, toxins, and mechanical forces, as well as providing the barrier to retain essential body fluids. The epidermis, which is composed of several layers beginning with the stratum corneum, is the outermost layer of the skin. The innermost skin layer is the deep dermis. The skin has multiple functions, including thermal regulation, metabolic function (vitamin D metabolism), and immune functions. FIG. 1 presents a schematic diagram of skin anatomy.

Epidermis

Closing the wound quickly and efficiently is a function of the epidermis. The epidermis provides a buffer zone against the environment. It provides protection from trauma, excludes toxins and microbial organisms, and provides a semi-permeable membrane, keeping vital body fluids within the protective envelope. Traditionally, the epidermis has been divided into several layers, of which two represent the most significant ones physiologically. The basal-cell layer, or germinative layer, is of importance because it is the primary source of regenerative cells. In the process of wound healing, this is the area that undergoes mitosis in most instances. The upper epidermis, including stratum and granular layer, is of importance because it is the area of formation of the normal epidermal-barrier function.

When the epidermis is injured, the body is subject to invasion by outside agents and loss of body fluids. Epidermal wounds heal primarily by cell migration. Clusters of epidermal cells migrate into the area of damage and cover the defect. These cells are phagocytic and clear the surface of debris and plasma clots. Repair cells originate from local sources that are primarily the dermal appendages and from adjacent intact skin areas. Healing occurs rapidly, and the skin is regenerated and is left unscarred. Blisters are examples of epidermal wounds. They may be small vesicles or larger bullae (blisters greater than 1 cm in diameter).

Stratum Corneum and the Acid Mantle

Stratum corneum is an avascular, multilayer structure that functions as a barrier to the environment and prevents transepidermal water loss. Studies have shown that enzymatic activity is involved in the formation of an acid mantle in the stratum corneum. Together, the acid mantle and stratum corneum make the skin less permeable to water and other polar compounds, and indirectly protect the skin from invasion by microorganisms. Normal surface skin pH is between 4 and 6.5 in healthy people; it varies according to area of skin on the body. This low pH forms an acid mantle that enhances the skin barrier function. Damage of the stratum corneum increases the skin pH and, thus, the susceptibility of the skin to bacterial skin infections.

Other Layers of the Epidermis

Other layers of the epidermis below the stratum corneum include the stratum lucidum, stratum granulosum, stratum germinativum, and stratum basale. Each contains living cells with specialized functions (FIG. 2). For example melanin, which is produced by melanocytes in the epidermis, is responsible for the color of the skin. Langerhans cells are involved in immune processing.

Dermal Appendages

Dermal appendages, which include hair follicles, sebaceous and sweat glands, fingernails, and toenails, originate in the epidermis and protrude into the dermis hair follicles and sebaceous and sweat glands contribute epithelial cells for rapid reepithelialization of wounds that do not penetrate through the dermis (termed partial-thickness wounds). The sebaceous glands are responsible for secretions that lubricate the skin, keeping it soft and flexible. They are most numerous in the face and sparse in the palm of the hands and soles of the feet. Sweat gland secretions control skin pH to prevent dermal infections. The sweat glands, dermal blood vessels, and small muscles in the skin (responsible for goose pimples) control temperature on the surface of the body. Nerve endings in the skin include receptors for pain, touch, heat, and cold. Loss of these nerve endings increases the risk for skin breakdown by decreasing the tolerance of the tissue to external forces.

The basement membrane both separates and connects the epidermis and dermis. When epidermal cells in the basement membrane divide, one cell remains, and the other migrates through the granular layer to the surface stratum corneum. At the surface, the cell dies and forms keratin. Dry keratin on the surface is called scale. Hyperkeratosis (thickened layers of keratin) is found often on the heels and indicates loss of sebaceous gland and sweat gland functions if the patient is diabetic. The basement membrane atrophies with aging; separation between the basement membrane and dermis is one cause for skin tears in the elderly.

Dermis

The dermis, or the true skin, is a vascular structure that supports and nourishes the epidermis. In addition, there are sensory nerve endings in the dermis that transmit signals regarding pain, pressure, heat, and cold. The dermis is divided into two layers: the superficial dermis consists of extracellular matrix (collagen, elastin, and ground substances) and contains blood vessels, lymphatics, epithelial cells, connective tissue, muscle, fat, and nerve tissue. The vascular supply of the dermis is responsible for nourishing the epidermis and regulating body temperature. Fibroblasts are responsible for producing the collagen and elastin components of the skin that give it turgor. Fibronectin and hyaluronic acid are secreted by the fibroblasts.

The second layer, the deep dermis, is located over the subcutaneous fat; it contains larger networks of blood vessels and collagen fibers to provide tensile strength. It also consists of fibroelastic connective tissue, which is yellow and composed mainly of collagen. Fibroblasts are also present in this tissue layer. The well-vascularized dermis withstands pressure for longer periods of time than subcutaneous tissue or muscle. The collagen in the skin gives the skin its toughness. Dermal wounds, e.g., cracks or pustules, involve the epidermis, basal membrane, and dermis. Typically, dermal injuries heal rapidly. Cracks in the dermis can exude serum, blood, or pus, and lead to formation of clots or crusts. Pustules are pus-filled vesicles that often represent infected hair follicle.

One nonlimiting example of an effect on the skin surface is formation of a film.

Film formation may be protective (e.g., sunscreen) and/or occlusive (e.g., to provide a moisturizing effect by diminishing loss of moisture from the skin surface). One nonlimiting example of an effect within the stratum corneum is skin moisturization, which may involve the hydration of dry outer cells by surface films or the intercalation of water in the lipid-rich intercellular laminae; the stratum corneum also may serve as a reservoir phase or depot where topically applied substances accumulate due to partitioning into or binding with skin components.

### Example 1. Safety Studies, LMWHA (miniHA™)

(1) Cytotoxicity Testing of miniHA™ Molecular Weight 8,300 Da

### Materials:

Epithelial cell line: Murine fibroblast cell line L929 (Chinese Academy of Science Type Culture Collection).

LMWHA, molecular weight 8,300 Da, degraded by Bacillus hyaluronidase, provided by Bloomage Freda Biopharm Co., Ltd (“mini-HA”).

HA-Oligo (molecular weight 8000), manufactured by chemical degradation.

### Methods

Two preparations of LMWHA were compared. LMWHA, molecular weight 8,300 Da, enzymatically degraded by Bacillus hyaluronidase, was compared to HA-Oligo (molecular weight 8000), manufactured by chemical degradation.

Different concentrations of Mini-HA or HA-Oligo in solution were added to monolayer cultures of L929 fibroblasts in complete medium to achieve final concentrations of 0.25%, 0.5%, 1%, 2% and 3% (w/v) The cultures were then incubated in a moist atmosphere at 37° C., 5% CO₂. Test samples were as follows: (1) complete medium; (2) complete medium+miniHA™; (3) complete medium+HA-oligo.

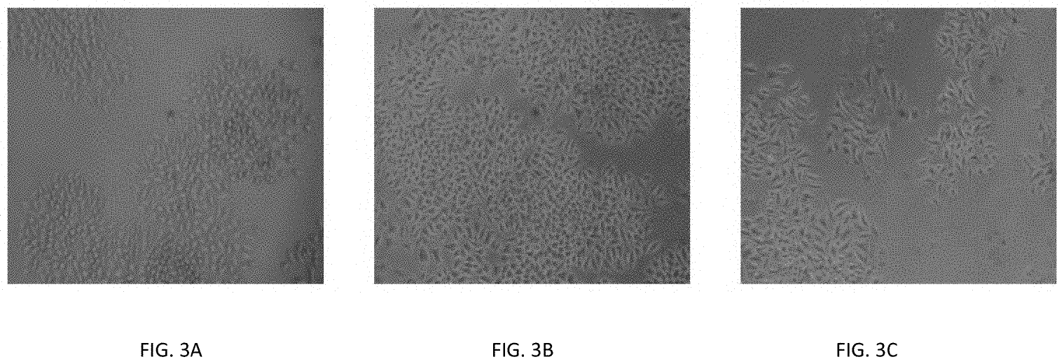

FIG. 3A-FIG. 3C show micrographs of an L929 fibroblast cytotoxicity test. FIG. 3A, complete medium (control); FIG. 3B, complete medium plus miniHA™; FIG. 3C, complete medium+HA-Oligo. No morphological cytotoxicity was observed.

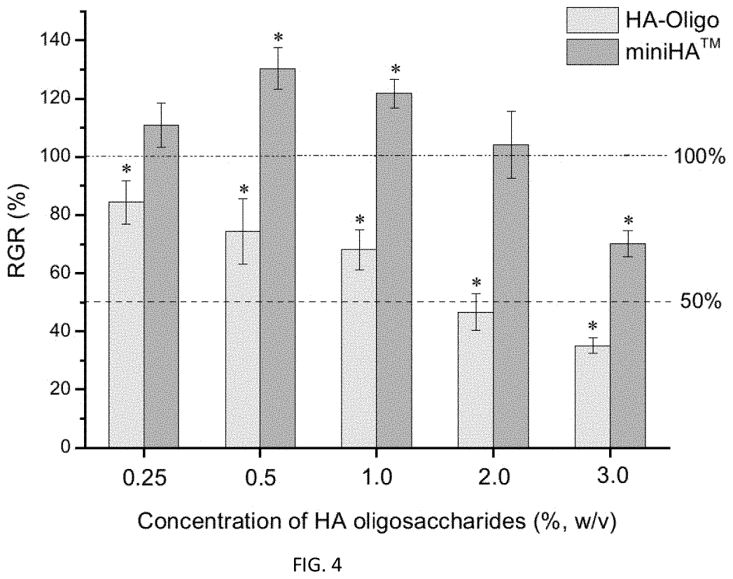

A plot of relative growth rate (RGR) versus concentration of HA oligosaccharides (%, w/v) added to in vitro cultures of L929 fibroblasts is shown in FIG. 4. The results indicate that there were no negative effects on cell proliferation when less than 2% (w/v) miniHA was added to the culture medium. Based on cytotoxicity grades of the U.S. Pharmacopoeia (toxic=relative growth rate (RGR)<50%), miniHA™ at a concentration of 3% (w/v) would be considered non-toxic. In contrast, when 2% (w/v) of HA-Oligo was added to the culture medium, significant cytotoxicity was observed. Such cytotoxicity has been attributed to byproducts of acid hydrolysis, such as furan-like and cyclopentanone derivatives. [See Smejkalova, D. et al. Carbohydrate Polymers (2012) 88: 1425-34].

(2) Skin Patch Testing of miniHA™

0.5 ml of a 1% solution of miniHA™ was applied to the flexible forearm (5×5 cm²) of 20 healthy volunteers twice a day for seven days and the cutaneous reaction observed. Results are shown in Table 4.

TABLE 4
Skin Patch Testing Results.

| Degree | Grade | Cutaneous reaction | Volunteers |
|---|---|---|---|
| − | 0 | Negative | 30 |
| ± | 1 | Weak erythema/Xerosis cutis/wrinkle | 0 |
| + | 2 | Erythema/edema/papula/ | 0 |
| | | wheal/desquamation/cracks | |
| ++ | 3 | Obvious erythema/edema/phlycten | 0 |
| +++ | 4 | Severe erythema/edema/bleb/erosion/ | 0 |
| | | hyperpigmentation or | |
| | | hypopigmentation/acne kind change | |

Conclusion: the cutaneous reactions of all of the volunteers to miniHA was negative.

Based on the results of the cytotoxicity test and the skin patch test, it can be concluded that miniHA™ is a safe cosmetic ingredient.

### Example 2: Evaluation of the Effects of LMWHA (miniHA™) and HMWHA on Hydration/Moisture Retention of Human Skin

Skin hydration is mostly linked to the skin barrier function and is important for maintaining a healthy skin barrier. The role of a moisturizing skin care product is to act on skin surface to physically limit water loss (occlusive effect) or to act at the cellular level.

Background A dielectric constant, also called relative permittivity or specific inductive capacity, is a property of an electrical insulating material (a dielectric) equal to the ratio of the capacitance of a capacitor filled with the given material to the capacitance of an identical capacitor in a vacuum without the dielectric material. The insertion of the dielectric between the plates of, for example, a parallel-plate capacitor always increases its capacitance, or ability to store opposite charges on each plate, compared with this ability when the plates are separated by a vacuum. If C is the value of the capacitance of a capacitor filled with a given dielectric and C₀ is the capacitance of an identical capacitor in a vacuum, the dielectric constant, symbolized by the Greek letter kappa, κ, is simply expressed as κ=C/C0. The value of the static dielectric constant of any material is always greater than 1, its value for a vacuum. The value of the dielectric constant at room temperature (25 C) is 1.00059 for air, and 78.2 for water.

The electrical properties of the skin are dependent on the water content of the stratum corneum of the epidermis. Epidermal hydration can be assessed by measuring electrical capacitance with the help of a Corneometer CM 825. The principle of the method is based on the difference between the dielectric constant of water and other substances by measuring the capacitance of a dielectric medium. (Constantin, M-M et al. “Skin hydration assessment through modern non-invasive bioengineering technologies. (2014) Maedica 9(10: 33-38). Any change in the dielectric constant subsequent to the variation in skin surface hydration leads to an impaired calculated capacitance of a capacitor.

Generally, the range of variation of the values of skin hydration degree is between 0-130 arbitrary units (AU). In standard working conditions (T°=20-22° C., humidity 40-60%), the variations of the values of skin hydration degree for the middle area of the front side of the forearm are the following: under 30 AU—very dry, between 30 and 45 AU—dry, 45 AU—sufficiently hydrated.

The advantages of this method are: very short measurement time (one second), high reproducibility of measurements and lack of galvanic contact between the measured area and the measuring apparatus (the results obtained are not influenced by ion conductivity or polarisation effects). In addition, the modern electronics of the probe enables temperature stability and excludes the interference of the base capacity and any power supply fluctuations. Compared to other methods, the preparations applied to the skin exert very little influence on the measurements, while being able to detect the slightest changes in the hydration level.

Method. Sample (3.0±0.01 mg/cm²) was applied on the left (control) and right (test sample) forearm (4×4 cm²) of 30 healthy volunteers ranging from 20-50 years of age. The degree of skin hydration after topical application was determined at multiple time points, i.e., at 1 h, 2 h, 4 h, 6 h and 8 h and compared to a control (before application).

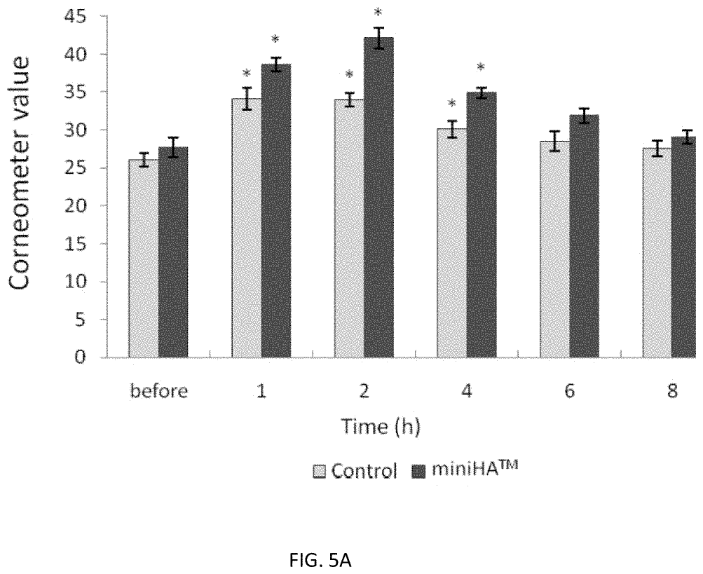

FIG. 5A shows the moisture retention capacity of miniHA™ molecular weight 8,300 Da, compared to a control. FIG. 5B shows the moisture retention capacity of high molecular weight hyaluronic acid, molecular weight 1,170 kDa, compared to a control. The data show that miniHA™ has the same capacity for moisture retention as does the high molecular weight HA tested.

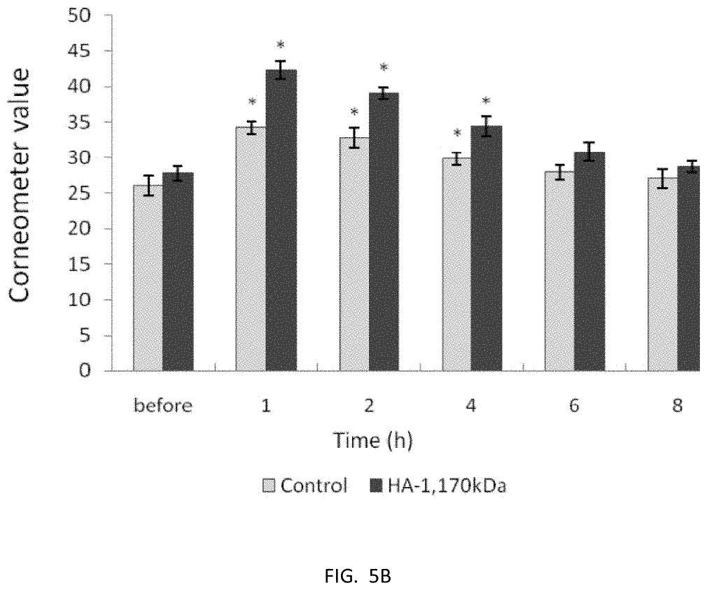

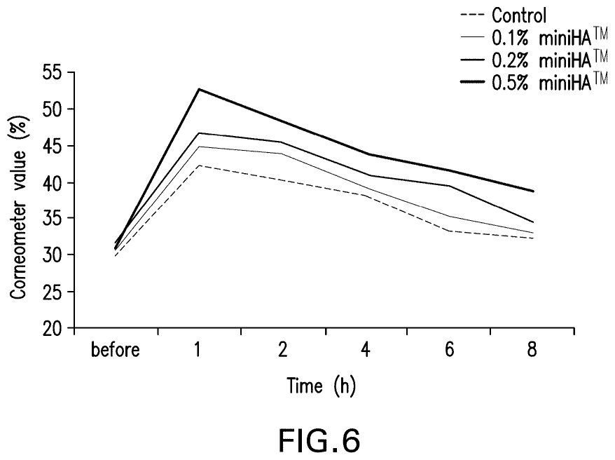

FIG. 6 is a graph of corneometer value (%) vs. time for 0.1% miniHA, 0.2% miniHA and 0.5% HA. As shown, the higher concentration of miniHA, i.e., 0.5% miniHA™, has a better moisture retention capacity than either 0.1% miniHA or 0.2% miniHA.

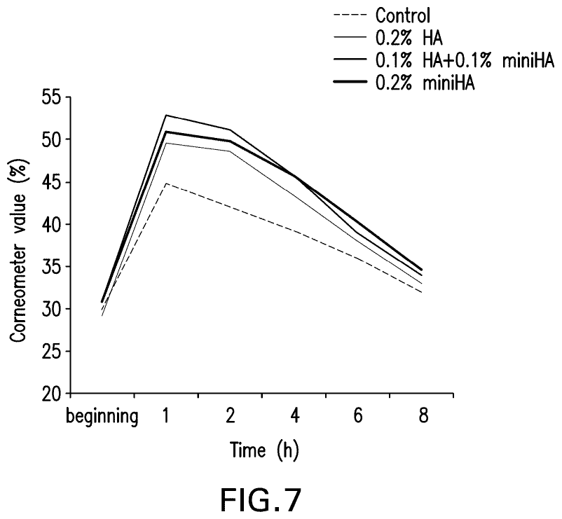

FIG. 7 is a graph of corneometer value (%) vs. time for 0.2% HA (molecular weight 1,630,000 DA); 0.2% miniHA; and 0.1% HA+0.1% miniHA. As shown, when miniHA™ was used together with HMWHA, the moisturizing effect was greater than for each alone, indicating that the combination of miniHA and HA together have a synergistic effect on moisture retention capacity of human skin. The molecular weight of mini-HA is so small (less than 10 kDa) that it can penetrate into the skin, and therefore is not sticky even in high concentration.

### Impact of Molecular Weight of HA on Moisturizing Effect and TEWL

Background. The outer layer of the epidermis, the stratum corneum (SC), contributes to skin barrier properties and has many protective functions (Alexander, H. et al. “Research Techniques made simple: transepidermal water loss measurement as a research tool. J. Investigative Dermatol. (2018) 138 (11): 2295-2300, citing Elias, P M Skin barrier function. Curr. Allergy Asthma Rep. (2008) 8: 299-305), including contribution to the control of transcutaneous water loss. The movement of water across the SC is primarily controlled by flattened corneocytes surrounded by hydrophobic bilamellar lipids including ceramides, cholesterol, and free fatty acids. The permeability barrier function of the skin is critical, and its impairment leads to downstream signals that aim to restore barrier homeostasis. Transepidermal water loss (TEWL) is the amount of water that passively evaporates through skin to the external environment due to water vapor pressure gradient on both sides of the skin barrier. It is a measure of skin water barrier status that has been validated in both humans and mice by correlating TEWL with absolute water loss determined gravimetrically (Id., citing Fluhr, J W. et al. Transepidermal water loss reflects permeability barrier status: validation in human and rodent in vivo and ex vivo models. Exp. Dermatol. (2006) 15: 483-92). In addition to gauging water barrier function, in vivo TEWL measurements consistently correlate with the percutaneous absorption of topically applied compounds (Id., citing Levin, J. and Maibach, H. The correlation between transepidermal water loss and percutaneous absorption: an overview. J. Control Release (2005) 103: 291-99). As such, TEWL measurements can be seen as an indirect measure of skin permeability (both inside to outside and outside to inside), which is a function of skin barrier status. A stronger skin barrier, characterized by larger surface corneocytes, an increased number of corneocyte layers (increased path length across the SC), and/or improved inter-corneocyte lamellar lipid matrices are linked to reduced TEWL (Id., citing Damien, F. and Boncheva, M. The extent of orthorhombic lipid phases in the stratum corneum determines the barrier efficiency of human skin in vivo. J. Invest. Dermatol. (2010) 130: 611-14).

TEWL is not measured directly, but inferred from measuring the change (or flux) in water vapor density at the skin surface compared with a point farther away from the skin (Id., citing Nilsson, G E. Measurement of water exchange through skin. Med. Biol. Eng. Comput. (1977) 15: 208-16). If water loss across the SC were zero, then the humidity in the air adjacent to the skin surface would be the same as ambient humidity. As water loss across the SC increases, the humidity next to the skin surface rises above ambient humidity. This creates a humidity gradient above the skin surface that is proportional to the SC water loss (Id., citing Imhof, R E et al. Closed-chamber transepidermal water loss measurement: microclimate, calibration and performance. Int. J. Cosmet. Sci. (2009) 31: 97-118). Water vapor density measurements are taken over a fixed area of SC in a fixed time period, and the units for TEWL are stated as grams of water per square meter per hour (g·m-2·h-1).

Materials: miniHA™ (Molecular weight 8 kDa, Batch 1012181); hyaluronic acid, molecular weight 270 kDa (HA-270 kDa, batch 1103031); hyaluronic acid molecular weight 1630 kDa (HA-1630 kDa).

Vehicle: cream.

Instrument: Corneometer CM825; Tewanmeter TM300.

Subjects: 30 healthy volunteers, 30-50 years of age.

Method: 3.0±0.1 mg/cm2 of control and test samples were applied on the left and right forearm respectively (4×4×m2). Skin moisture of the test area was measured before and after application of the samples at 1 h, 2 h, 4 h, 6 h and 8 h.

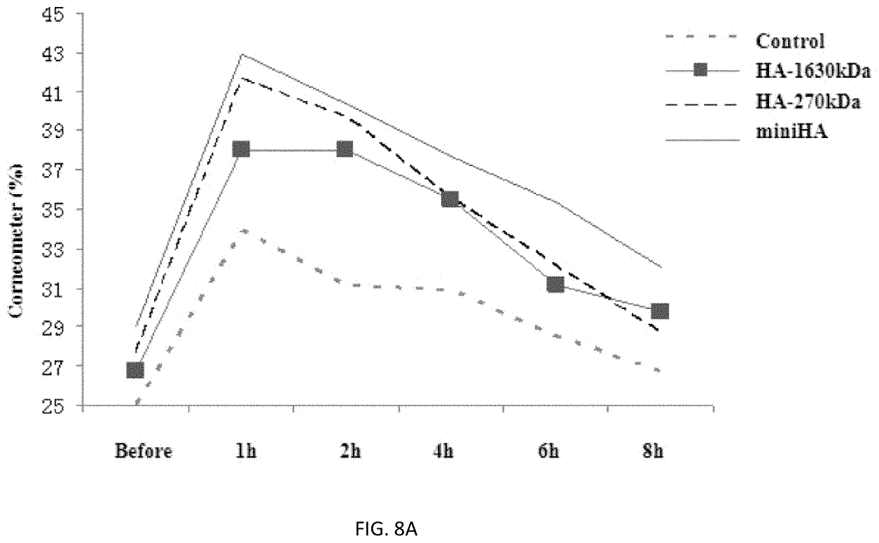

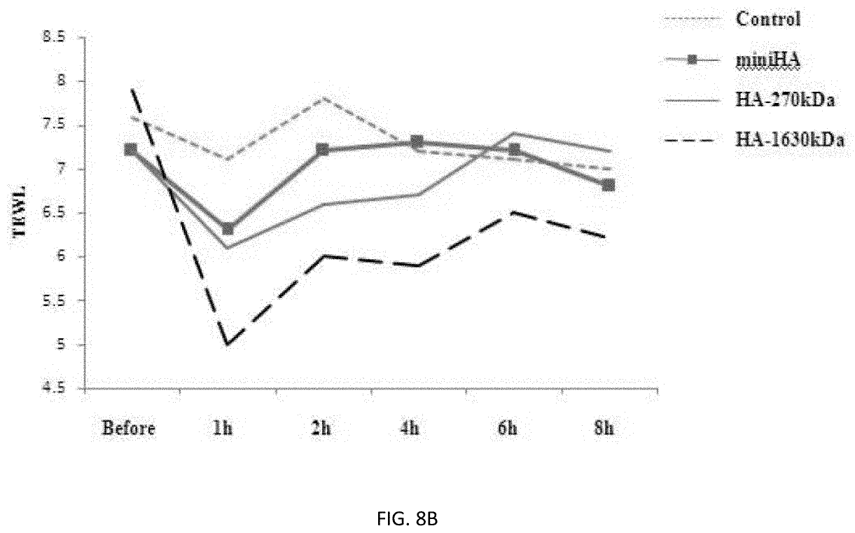

FIG. 8A and FIG. 8B shows results of the moisture retention test (FIG. 8A) and the TEWL test (FIG. 8B). FIG. 8A is a graph of corneometer value (%) vs. time for 0.1% mini HA, 0.1% HA-1630 kDa, and for HA-270 kDa. As shown, the lower the molecular weight of HA, the better the moisture retention was. The miniHA group had the highest value of skin hydration at each time point. FIG. 8B is a graph of transepidermal water loss (TEWL) versus time for 0.1% mini HA, 0.1% HA-1630 kDa, and for 0.1% HA-270 kDa. The data show that the higher molecular weight of HA, the less the skin water was reduced.

When miniHA (8 kDa) and high molecular weight HA (HA-270 kDA) are formulated together, the moisturizing effect is better.

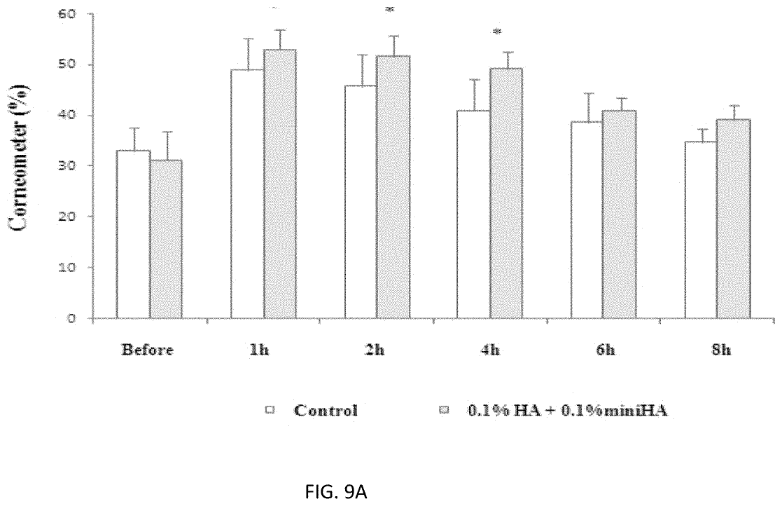

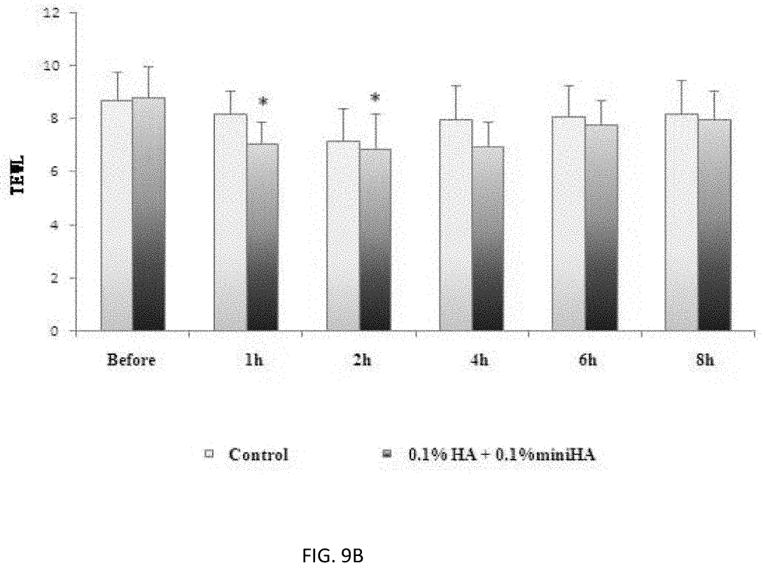

FIG. 9A and FIG. 9B show the results of the moisture retention test (FIG. 9A) and the TEWL test (FIG. 9B) when 0.1% miniHA and 0.1% HA-270 kDA are combined.

Results presented in FIG. 9A are bar graphs showing moisture showing moisture retention capacity as measured by corneometer before topical application of the sample, and at 1 h, 2 h, 4 h, 6 h and 8 h after application. Hydration before was about the same in both control and treated subjects. Hydration improved at each time point after administration of this test material.

FIG. 9B shows bar graphs of transepidermal water loss (TEWL) versus time for 0.1% mini HA+0.1% HA-270 kDa. High molecular weight HA has good TEWL reducing ability by forming a film on the surface of the skin. Mini HA can penetrate into the skin, hydrating the epidermis and dermis. Together, the moisturizing effect is better than either alone.

### Example 3. In Vitro Evaluation of Penetrability of miniHA Through In Vitro Reconstructed Human Skin

### Definitions and Background

The term “full thickness skin” as used herein refers to skin consisting of the complete epidermis and dermis.

The term “partial-thickness skin” as used herein refers to skin consisting of the entire dermis and only partial dermis.

“Reconstructed human epidermis” model. In order to produce and maintain the vital epidermal barrier, the keratinocyte, the main cell type in this tissue, undergoes proliferation and differentiation. During the progressive terminal maturation of the keratinocyte, its cellular morphology changes from typically cuboidal in the undifferentiated proliferative cells anchored on the epidermo-dermal junction in the basal layer, into a squamous morphology in the dead cells of the cornified layer. Between these layers, morphological changes mean taking shape of a prickle cell within the spinous layer and intracytoplasmic accumulation of dark structures, named keratohyalin granules, inside the granular layer, underlying the cornified barrier. [Poumay, Y, Coquettte, A. “Modeling the human epidermis in vitro: tools for basic and applied research.” Arch. Dermatol. Res. (2007) 298 (8): 361-9].

The typical epidermal organization into four layers reveals that inside the keratinocytes, the differentiation program is intended to produce the epidermal barrier. The appearance of different layers simply results from progressive maturation of this cell type inside the epidermis. Because desquamation, i.e. detachment of cornified keratinocytes, occurs regularly from the surface of the epidermis, a constant proliferation of cells in the lowest basal layer must be regulated in order to guarantee homeostasis of the epidermal tissue, i.e. an equilibrium between the number of cells lost from the surface of the body and the number of new keratinocytes produced deeply within the basal layer.

Keratinocytes can be cultured in immersed conditions as a monolayer or as stratifying layers, but we will see below that it can further be grown in conditions where the cultured cells reconstruct the basis of an epidermis with three dimensional organization and production of a cornified barrier when the surface of the culture is exposed to the air [Id., citing Prunieras, M. et al. Methods for cultivation of keratinocytes with an air-liquid interface. J. Invest. Dermatol. (1983) 81: 28s-33s].

It is possible to get full differentiation in vitro simply by raising cells up to the air-liquid interface [Id., citing Prunieras, M. et al. “Methods for cultivation of keratinocytes with an air-liquid interface.” J. Invest. Dermatol. (1983) 81: 28s-33s]. The interface with air stimulates in keratinocytes the synthesis of profillagrin and thus the appearance of the granular phenotype when keratohyalin granules develop. These granules never appear in immersed culture conditions and seem to be the missing link that allows the final gain-of-function for keratinocytes during cell cornification. In such culture conditions, keratinocytes located at the top of the granular layer scarify and leave their fully differentiated cell skeleton (represented by the aggregated intermediate filaments) or cell shell (represented by the cornified envelope formed after activation of transglutaminase) to the human body in order to maintain the superficial barrier.

Growth at the air-liquid interface in vitro means feeding the epidermal cells from the bottom of the reconstructed tissue, through the basal layer. This is close to the situation in vivo, but must be done without any blood circulation. The 3D-reconstruction of the epidermis has been adapted to basal inert substrates such as porous filters [Id., citing Rosdy, M., Claus, L C. “Terminal epidermal differentiation of human keratinocytes grown in chemically defined medium on inert filter substrates at the air-liquid interface.” J. Invest. Dermatol. (1990) 95: 409-14]. Filters provide a solid mechanical support on which keratinocytes can attach through integrins, organize hemidesmosomes and then stratify thanks to the formation of adherens junctions and desmosomes in order to form the typical epidermal layers. These layers can then be easily exposed to the air-liquid interface when the culture medium is only present in the compartment under the filter. The diameter of the pores in the filter must be small enough in order to impede keratinocyte migration through the holes and an eventual colonization of the other side of the filter.

After 14 days in culture, a stratified epidermis has formed that closely resembles human epidermis in vivo. Morphologically, these cultures exhibit a well-stratified epithelium and cornified epidermis with significantly improved barrier function and metabolic activity. Differentiation markers, such as suprabasal keratins, integrin (34, integrin a6, fibronectin, involucrin, filaggrin, trichohyalin, type I, III, IV, V and VII collagen, laminin, heparin sulfate and membrane-bound transglutaminase have been found to be expressed similar to those of the epidermis.

### Materials

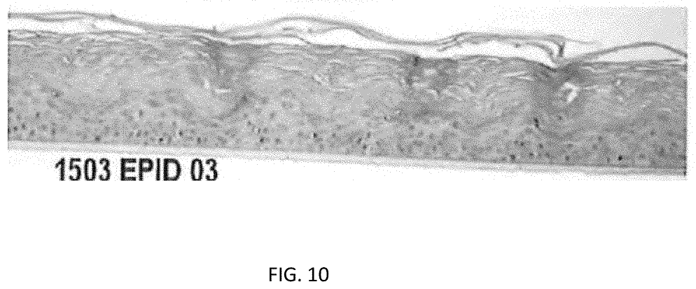

Human reconstructed epidermis (shown in FIG. 10) is defined as 0.5 cm² epidermis cultivated from human keratinocytes, reconstituted by airlifted culture on inert polycarbonate filers (0.4 μm).

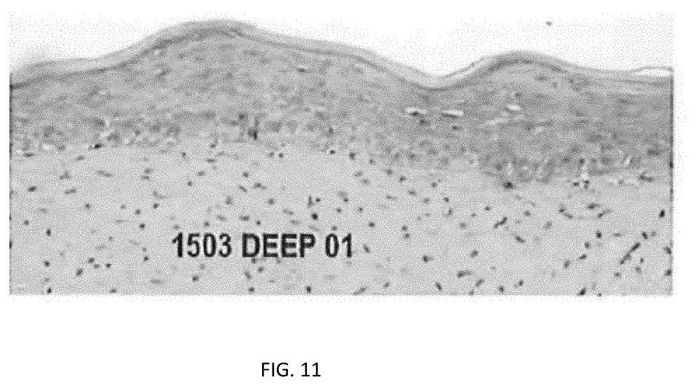

Human reconstructed full thickness skin (shown in FIG. 11) is defined as 0.5 cm² full thickness cultivated from human keratinocytes and collagen matrix with fibroblasts, reconstituted by airlifted culture on inert polycarbonate filters (0.4 μm).

### Methods

Summary. The aim of this study was to determine the penetration capability of sodium hyaluronate oligosaccharides (miniHA™, molecular weight <10,000 Da) through reconstructed human skin. In order to investigate the depth reached by the absorbed active, two parallel tests were performed: one on reconstructed human epidermis; the other on reconstructed human full thickness skin (epidermis+dermis).

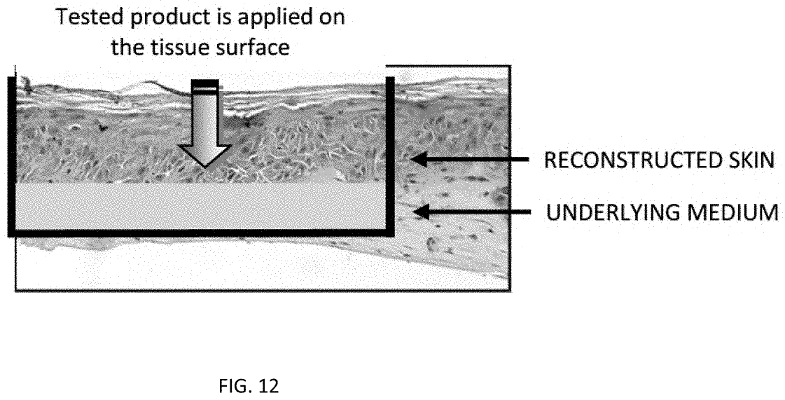

A schematic of the experimental system is shown in FIG. 12. The product applied on the surface penetrates the reconstructed tissue; a certain amount of the product is maintained by the tissue structure.

Tested product was applied on the surface of the tissue in aqueous solution at 0.5% and the hyaluronic acid absorbed by the tissues during the time was evaluated by ELISA. Absorption kinetics were constructed by evaluation of the hyaluronic acid absorbed by epidermis and full thickness skin (epidermis+dermis) in the following experimental times representing time after product application: T30 min, T1h, T2h, T4h, T8h, T24h. At the end of each experimental period, the treated tissues were washed, homogenized and used to determine the hyaluronic acid amount absorbed in their structure.

Protocol:

Tested products were diluted in 0.01 M phosphate buffer (pH 7.4) at 0.5% and the obtained solution applied on the tissue surface. Two (2) units were used for each experimental condition.

Test Procedure

32 μl/cm² of solution was applied on two (2) tissue units for 30 minutes, 1 hour, 2 hours, 4 hours, 8 hours, and 24 hours. At the end of the test period, treated tissues were washed with phosphate buffer and homogenized in order to determine the amount of hyaluronic acid absorbed. Hyaluronic acid was measured by ELISA. The baseline amount of hyaluronic acid of untreated tissues was automatically subtracted during the test session.

Hyaluronic acid content, expressed in ng, was calculated for each collected sample at each experimental time. The percentage of product absorbed was calculated according to the hyaluronic acid quantity applied on the tissue. Absorption into the dermis is mathematically calculated by the difference between the total absorption into full thickness skin and epidermis. Results are expressed as mean data±standard deviation.

The following tables 5, 6, and 7 show the data obtained from the hyaluronic acid dosage in the epidermis and full thickness models treated with miniHA™

TABLE 5
HA-Oligo degraded by hyaluronidase (miniHA ™) into human
reconstructed dermis. Applied quantity: 80000 ng miniHA.

| | Hyaluronic acid (ng) (mean ± | |
|---|---|---|
| Time (T) | standard deviation) | % absorption |
| 30 min | 8889.3 ± 270.5 | 11.1% |
| 1 h | 18642.4 ± 284.1 | 23.3% |
| 2 h | 23836.6 ± 1252.3 | 29.8% |
| 4 h | 26726.5 ± 2178.7 | 33.4% |
| 8 h | 27175.7 ± 1465.3 | 34.0% |
| 24 h | 29406.0 ± 1138.6 | 36.8% |

TABLE 6
HA-Oligo degraded by hyaluronidase (mini-HA ™)
into humanreconstructed full thickness.
(epidermis + dermis). 80000 ng mini-HA was applied.

| | Hyaluronic acid (ng) | |
|---|---|---|
| Time | (mean ± standard deviation) | % absorption |
| 30 min | 10122.4 ± 1103.9 | 12.7% |
| 1 h | 28964.8 ± 2820.1 | 36.2% |
| 2 h | 35359.3 ± 3028.7 | 44.2% |
| 4 h | 42847.2 ± 4024.3 | 53.6% |
| 8 h | 48564.9 ± 5930.3 | 60.7% |
| 24 h | 55564.3 ± 7737.5 | 69.5% |

TABLE 7
shows HA-oligo degraded by hyaluronidase
(mini-HA ™) into dermis The results were mathematically
calculated by subtraction of the full thickness
and epidermis absorption data.

| Time | % absorption |
|---|---|
| 30 min | 1.5% |
| 1 h | 12.9% |
| 2 h | 14.4% |
| 4 h | 20.2% |
| 8 h | 26.7% |
| 24 h | 32.7% |

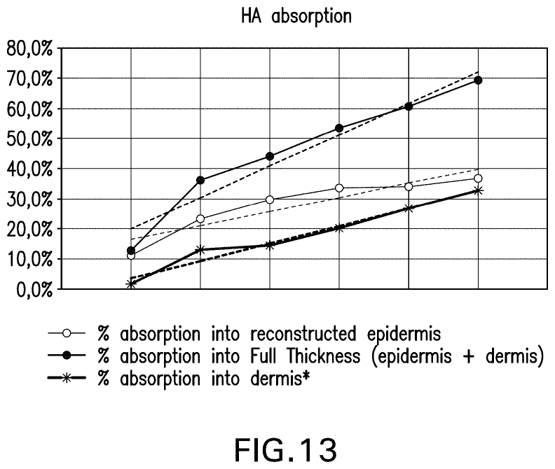

FIG. 13 is a graph of % HA absorption vs. time for reconstructed epidermis; full thickness reconstructed skin; and dermis. The dermis results were mathematically calculated by subtraction of the full thickness and epidermis absorption data.

The results of this penetration test on human in vitro reconstructed skin show that miniHA hyaluronic acid is capable of penetrating the reconstructed tissue during the monitored experimental period. There is a constant time-dependent absorption gradient through the full thickness skin, reaching 69.5% absorption.

The results also show that the hyaluronic acid distributes through epidermis and dermis. The absorption in the epidermis has a linear pattern until T=4 hours, while it reaches a plateau from 4-24 hours (i.e., maximum absorption potential is reached). In the dermis, HA absorption has a linear pattern during the monitored experimental period. At the end of the monitored experimental time, HA has equally distributed between epidermis and dermis.

### Example 4. In Vitro Evaluation of the Wound Healing Capacity of Three Formulations after Treatment of Human Keratinocytes

### Summary

The goal of this study was to determine the regenerating and stimulating effects of test samples compared to human epidermal growth factor (hEGF) after a 24 hr treatment of human keratinocytes (HaCaT) using a wound-healing (Scratch) Assay.

### Test Samples.

Test samples were stored at room temperature. Dilutions were freshly prepared prior to use in cell culture.

TABLE 8
MC-21-198A (RS-0198A) Final Formulation of
LMHWA + HMWHA + CBD isolate

| | PERCENTAGE |
|---|---|
| INGREDIENT | (BASED ON WEIGHT) |
| Aloe Barbadensis Leaf Juice | 94.75 |
| Water, Arginine Levulinate, Arginine Anisate | 2.00 |
| Maltodextrin, Cannabidiol | 1.50 |
| Lysolecithin, Scloerotium Gum, Xanthan | 0.75 |
| Gum, Pullan | |
| Sodium Hyaluronate | 0.50 |
| Hydrolyzed Sodium Hyaluronate | 0.25 |
| Lactic Acid | 0.15 |
| Phytic Acid | 0.10 |

TABLE 9
MC21-198B (RS-0198B): HMWHA and LMWHA in ratio of 2:1

| | PERCENTAGE |
|---|---|
| INGREDIENT | (BASED ON WEIGHT) |
| Aloe Barbadensis Leaf Juice | 96.25 |
| Water, Arginine Levulinate, Arginine Anisate | 2.00 |
| Lysolecithin, Scloerotium Gum, Xanthan | 0.75 |
| Gum, Pullan | |
| Sodium Hyaluronate | 0.50 |
| Hydrolyzed Sodium Hyaluronate | 0.25 |
| Lactic Acid | 0.15 |
| Phytic Acid | 0.10 |

TABLE 10
MC20155C (RS01221) CBD only (1.0-5 wt %
of isolate comprising 20% decarboxylated CBD)

| | PERCENTAGE |
|---|---|
| INGREDIENT | (BASED ON WEIGHT) |
| Aloe Barbadensis Leaf Juice | 95.5 |
| Water, Arginine Levulinate, Arginine Anisate | 2.00 |
| Maltodextrin, Cannabidiol | 1.50 |
| Lysolecithin, Scloerotium Gum, Xanthan | 0.75 |
| Gum, Pullan | |
| Lactic Acid | 0.15 |
| Phytic Acid | 0.10 |

### Method:

Reagents: HaCaT cells, distilled water (Braun); DMEM medium 1 g/L glucose (GIBCO); phosphate buffered saline Sigma); trypan blue solution (BioRad); DMSO (Sigma-Aldrich); Trupsin (Sigma); Penicillin-Streptomycin (GIBCO); L-glutamine (Sigma); EGF (Sigma-Aldrich).

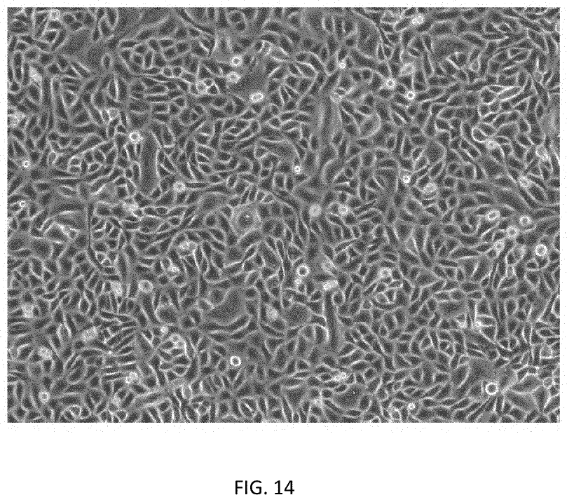

FIG. 14 is a microscopic image of human heratinocytes (HaCaT line) in culture that were used in the study.

An MTT cell viability assay was performed to select two or three working concentrations of the three test samples for the following cell culture treatment.

An in vitro scratch assay was used to study cell migration in vitro. [Todaro, G J et al. The initiation of cell division in a contact-inhibited mammalian cell line. J. Cell Physiol. (1965) 66: 325-33]. This method is based on the observation that, upon creation of a new artificial gap or “scratch”, on a confluent cell monolayer, the cells on the edge of the newly created gap will move toward the opening to close the “scratch” until new cell-cell contacts are established. One of the major advantages of this simple method is that it mimics to some extent migration of cell in vivo. For example, removal of part of the endothelium in blood vessels will induce migration of endothelial cells (ECs) into the denuded area to close the wound [Haudenschild, C. et al. Endothelial regeneration. II. Restitution of endothelial continuity. Lab. Invest. (1979) 41: 407-18]. Furthermore, the patterns of migration either as a loosely connected population (e.g., fibroblasts) or as sheets of cells (e.g., epithelial and EECs) also mimic the behavior of these cells during migration in vivo. The scratch assay is particularly suitable to study the regulation of cell migration by cell interaction with extracellular matrix (ECM) and cell-cell interactions.

For the wound-healing assay, HaCaT keratinocytes were seeded overnight in DMEM medium supplemented with 10% fetal bovine serum (FBS) at 80% confluence (190,000 cells per well. The next day, a 2 mm wound was made over the confluent monolayer using a 1 ml pipete tip. Test samples were added to the HaCaT cells, supplemented with 0.5% FBS. The untreated control was 0.5% FBS.

Images were taken before and after 24 hours of treatment, and wound area was quantified using ImageJ software. All data were statistically analyzed.

Results. Treatment for 24 hours with RS-0198A at 0.00001%, 0.0001% and 0.001% significantly enhanced wound healing by 63.4±15.7%, 61.4±17.0% and 61.9±15.7% compared to the untreated control. Treatment with RS-0198B at 0.001% for the same time period induced a wound healing effect with an increase of 53.7±15.7%, compared to the untreated control. RS012221 at 0.01% significantly improved wound healing in human keratinocytes by 51.7±16.9%, compared to the untreated control. The positive control hEGF at 20 ng/ml also boosted wound healing by 60.4±15.7% versus untreated control.

Conclusion. Each of RS-0198A, RS-0198b and RS12221 displayed regenerating and stimulating capabilities through significant improvement of wound healing to an extent similar to hEGF.

### Procedure

Cell Viability—MTT Assay

For seeding cells, cell number and viability were determined using Trypan-blue staining; cells were counted in a Bürker chamber under the microscope. For the MTT assay, ECVAM Guidelines as established in eCVAM Database Service on alternative Methods to Animal Experimentation (MTT assay protocol nr. 17) were followed.

HaCaT cells were cultured overnight at a 10,000 cells/well density in a 96-well plate in supplemented growth medium. 24 hours later, the culture medium was replaced with fresh medium with test samples RS-0198A, RS-0198B and RS-012221 at 8 different concentrations (3%, 1%, 0.3%, 0.1%, 0.03%, 0.01%, 0.003%, and 0.001%). The untreated control contained culture medium only. After 24 hours of incubation, the medium was removed, and MTT solution was added to each well. Plates were incubated at 37 C for 3 hours. MTT reactive was removed, and DMSO 100% was added to each well to solubilize formazan crystals prior to absorbance measurements at 550 nm and 620 nm as a reference on a scanning multiwall spectrophotometer.

Eight technical replicates per condition and 16 technical replicates for the untreated control were used. All data were statistically analyzed using ordinary one-way ANOVA. Statistical significance was set at p-value <0.05, 95% confidence. Absorbance values lower than those of control cells indicated a reduction in the rate of cell proliferation. Conversely, a higher absorbance rate indicated an increase in cell proliferation.

### Wound Healing

For seeding cells, cell numbers and viability were determined using Trypan-Blue staining; cells were counted in a Bürker chamber under the microscope. Human keratinocytes (HaCaT cells) were seeded overnight in DMEM medium supplemented with 10% fetal bovine serum (FBS).

Parallel lines were drawn at the bottom of the wells (3 mm separated lines outside the plate). These lines were used as boundaries for scratch images, thus providing an accurate analysis in each well. Cells were seeded at 190,000 cells/well to promote the formation of a monolayer and kept at 37 C for 24 h. Then, 2 mm-wide wounds were made in the confluent monolayer. Wells were washed once with PBS and refilled with DMEM+0.5% FBS. Basal images were taken immediately using a microscope. Table 11 shows test concentrations of each test sample that were added to each well. In addition, human epidermal growth factor (hEGF) at 20 ng/ml was included as a positive control, and 0.5% FBS as a basal control.

TABLE 11

| | Starting | | | |
|---|---|---|---|---|
| | Concentration | Dilutions tested (cytotoxicity) | | |
| hEGF | 20 ng/ml | | | |
| RS-0198A | | 0.00001% | 0.0001% | 0.001% |
| RS-0198B | | 0.0001% | 0.001% | 0.01% |
| RS-012221 | | 0.001% | 0.01% | |

Plates were placed in the 37° C. incubator and incubated for 24 h to allow the scratch to heal. Microscopic images are shown below. One biological replicate was performed and four technical replicates per condition were used.

Data analysis was performed using ImageJ software. Microscopic images are shown in Results. Statistical analysis (ordinary one-way ANOVA test) was performed to determine the significance between tested samples and basal control.

### Results, Cell Viability-MTT Assay

The effects of products RS-0198A and RS-0198B on cell viability were assessed through an MTT assay to determine non-toxic concentrations to be used in the subsequent wound healing study. The raw data is shown in Tables 11, 12 and 13.

TABLE 12
MTT assay raw data for product RS-0198A
RS-0198A

| Control | 3% | 1% | 0.3% | 0.1% | 0.03% | 0.01% | 0.003% | 0.001% | Control |
|---|---|---|---|---|---|---|---|---|---|
| 1.694 | 0.177 | 0.128 | 0.609 | 1.122 | 1.116 | 1.45 | 1.498 | 1.544 | 1.544 |
| 1.561 | 0.175 | 0.123 | 0.591 | 0.879 | 1.264 | 1.376 | 1.278 | 1.379 | 1.62 |
| 1.631 | 0.182 | 0.124 | 0.588 | 0.884 | 1.138 | 1.141 | 1.339 | 1.456 | 1.521 |
| 1.577 | 0.186 | 0.119 | 0.581 | 0.933 | 1.081 | 1.14 | 1.414 | 1.414 | 1.625 |
| 1.374 | 0.202 | 0.124 | 0.627 | 0.918 | 1.266 | 1.347 | 1.383 | 1.6 | 1.623 |
| 1.578 | 0.201 | 0.123 | 0.673 | 0.887 | 1.266 | 1.557 | 1.527 | 1.614 | 1.591 |
| 1.499 | 0.197 | 0.14 | 0.626 | 1.036 | 1.283 | 1.46 | 1.651 | 1.697 | 1.714 |
| 1.621 | 0.181 | 0.115 | 0.563 | 1.045 | 1.332 | 1.518 | 1.596 | 1.571 | 1.59 |

TABLE 13
MTT assay raw data for product RS-0198B
RS-0198B

| Control | 3% | 1% | 0.3% | 0.1% | 0.03% | 0.01% | 0.003% | 0.001% | Control |
|---|---|---|---|---|---|---|---|---|---|
| 1.342 | 0.887 | 0.821 | 1.15 | 1.242 | 1.377 | 1.47 | 1.446 | 1.556 | 1.7 |
| 1.371 | 0.806 | 0.89 | 0.888 | 1.001 | 1.226 | 1.33 | 1.491 | 1.437 | 1.667 |
| 1.116 | 0.857 | 0.812 | 0.98 | 1.123 | 1.253 | 1.345 | 1.466 | 1.468 | 1.813 |
| 1208 | 0.818 | 0.886 | 1.114 | 1.082 | 1.188 | 1.437 | 1.353 | 1.585 | 1.554 |
| 1.202 | 0.816 | 0.916 | 1.134 | 1.233 | 1.262 | 1.429 | 1.507 | 1.584 | 1.577 |
| 1.262 | 0.875 | 0.894 | 0.979 | 1.1 | 1.329 | 1.542 | 1.451 | 1.611 | 1.667 |
| 1.362 | 0.889 | 0.989 | 1.096 | 1.267 | 1.389 | 1.513 | 1.558 | 1.625 | 1.664 |
| 1.525 | 0.881 | 0.953 | 1.094 | 1.301 | 1.448 | 1.52 | 1.555 | 1.674 | 1.758 |

TABLE 14
MTT assay raw data for product 012221
012221

| Control | 3% | 1% | 0.3% | 0.1% | 0.03% | 0.01% | 0.003% | 0.001% | Control |
|---|---|---|---|---|---|---|---|---|---|
| 1.098 | 0.028 | 0.022 | 0.444 | 0.692 | 0.833 | 0.791 | 0.879 | 1.041 | 1.086 |
| 0.976 | 0.029 | 0.021 | 0.412 | 0.701 | 0.793 | 0.935 | 0.749 | 0.874 | 0.989 |
| 0.892 | 0.033 | 0.023 | 0.425 | 0.714 | 0.762 | 0.920 | 0.840 | 0.857 | 0.996 |
| 1.019 | 0.033 | 0.024 | 0.399 | 0.698 | 0.865 | 0.897 | 0.790 | 0.909 | 0.892 |
| 1.027 | 0.033 | 0.024 | 0.415 | 0.689 | 0.845 | 0.869 | 0.835 | 0.952 | 1.047 |
| 1.099 | 0.032 | 0.023 | 0.405 | 0.719 | 0.772 | 0.860 | 0.826 | 0.935 | 1.066 |
| 0.823 | 0.033 | 0.024 | 0.409 | 0.682 | 0.866 | 0.918 | 0.831 | 0.978 | 1.021 |
| 0.888 | 0.031 | 0.024 | 0.416 | 0.762 | 0.853 | 0.902 | 0.850 | 0.987 | 1.081 |

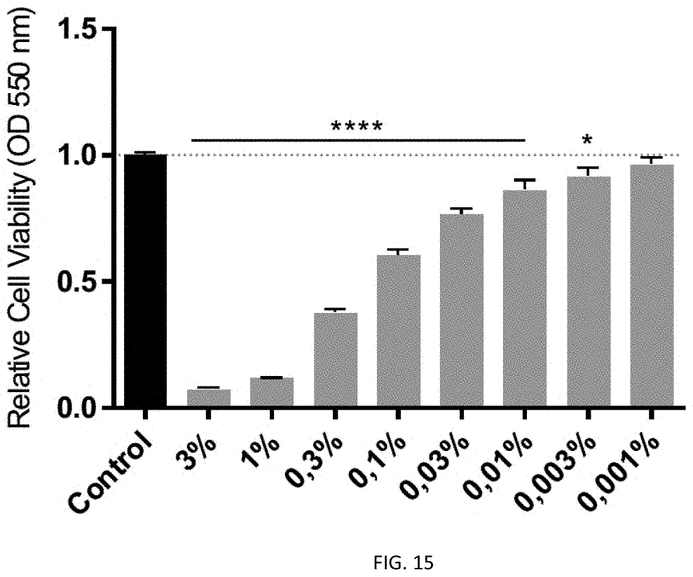

Cell viability results indicated that at concentrations equal to or lower than 0.001%, incubation of cells with RS-0198A was not cytotoxic for HaCaT after 24 hours FIG. 15 is a bar graph showing relative cell viability (OD 550 nm) versus RS-0198A at concentrations 3%, 1%, 0.3%, 0.1%, 0.03%, 0.01%, 0.003%, and 0.001%, compared to the untreated control group. **** represents statistical significance with p-value <0.0001. * represents statistical significance with p-value <0.05.

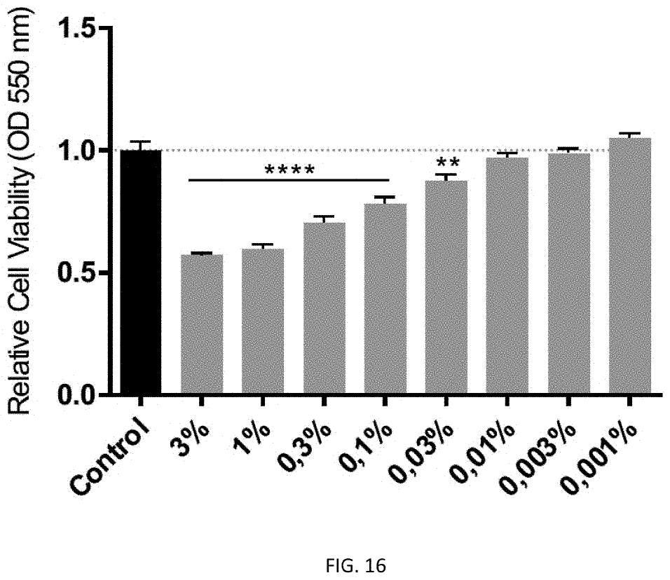

Cell viability results indicated that incubation of cells with RS-0198B at concentrations equal to or lower than 0.01% was not cytotoxic for HaCaT cells. FIG. 16 is a bar graph showing relative cell viability (OD 550 nm) versus RS-0198B at concentrations 3%, 1%, 0.3%, 0.1%, 0.03%, 0.01%, 0.003%, and 0.001%, compared to the untreated control group. **** represents statistical significance with p-value <0.0001. ** represents statistical significance with p-value <0.01.

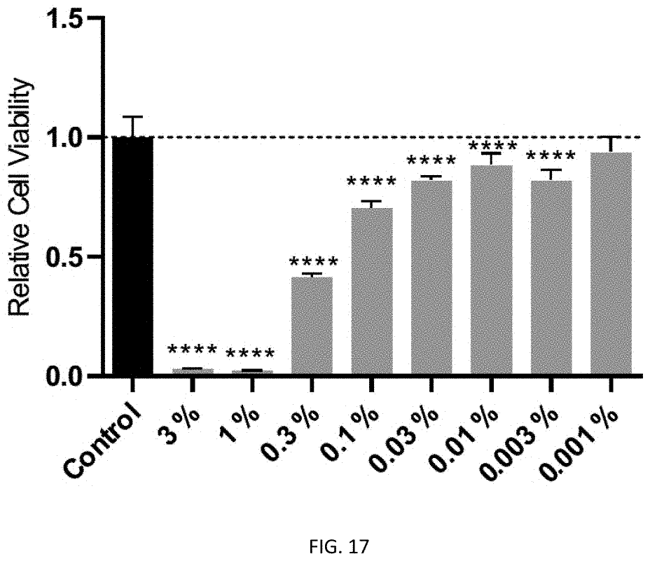

Cell viability results indicated that incubation of cells with RS-012221 at the lowest dose of 0.001% compared to the untreated control did not impair cell viability. FIG. 17 is a bar graph showing relative cell viability (OD 550 nm) versus RS-012221 at concentrations 3%, 1%, 0.3%, 0.1%, 0.03%, 0.01%, 0.003%, and 0.001%, compared to the untreated control group. **** represents statistical significance with p-value <0.0001.

### Wound Healing Assay

The percentage of wound area in each well at 24 hr was calculated by dividing the wound area at 24 hr and the wound area immediately after making the wound. The mean value for the four replicates in the Basal Control (untreated) group was obtained and used to normalize each measure in all of the samples and conditions. A bar graph was generated to represent the wound area or wound healing in each of the tested conditions.

Based on the MTT assay results, the following concentrations were selected for each test sample:

TABLE 15

| | Starting | | | |
|---|---|---|---|---|
| Test Sample | Concentration | Dilutions tested (wound healing) | | |
| RS-0198A | | .00001% | 0.0001% | 0.001% |
| RS-0198B | | 0.0001% | 0.001% | 0.01% |
| RS-012221 | | 0.001% | 0.01% | |

### Results

As shown in Table 16, treatment for 24 hr with RS-0198A, RS-0198B and EGF at 20 ng/ml significantly decreased wound area in cultures of human keratinocytes compared to the untreated control.

TABLE 16

| Test Sample | Dilution tested | Decreased wound area |
|---|---|---|
| RS-0198A | 0.00001% | 63.4 ± 15.7% |
| RS-0198A | 0.001% | 61.4 ± 17% |
| RS-0198A | 0.001% | 61.9 ± 15.7% |
| RS-0198B | 0.001% | 53.7 ± 15.7 |
| EGF | 20 ng/ml | 60.4 ± 15.7% |

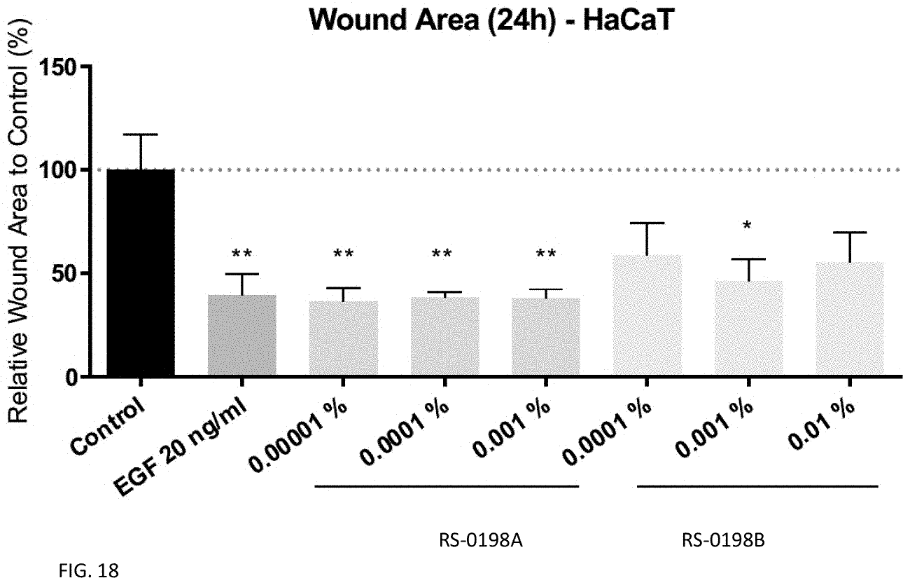

FIG. 18 is a bar graph representing wound area relative to an untreated control after treatment for 24 hours of human keratinocytes (HaCaT) with RS-0198a at 0.00001%, 0.0001%, 0.001% and RS-0198b at 0.0001%, 0.001%, and 0.01%. Human EGF at 20 ng/ml was included as a positive control. * Represents statistical significance with p-value <0.05. ** Represents statistical significance with p-value <0.01.

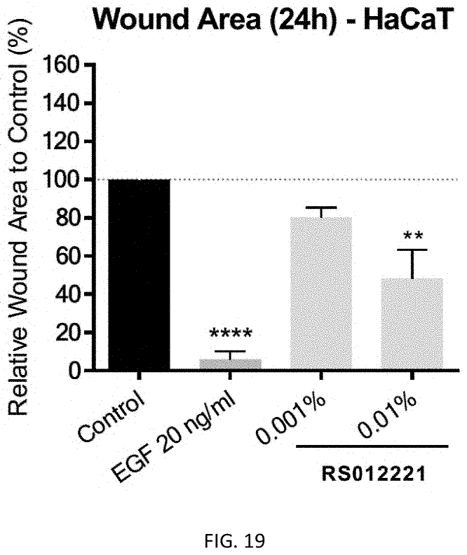

Results showed that treatment for 24 hr with RS012221 at 0.01% significantly decreased wound area in human heratinocytes by 51.7±16.9% compared to the untreated control. hEGF at 20 ng/ml reduced the wound area by 94.0±8.6%. FIG. 19 is a bar graph representing wound area relative to an untreated control after treatment for 24 hours of human keratinocytes (HaCaT) with RS012221 at 0.001% and 0.01% concentrations. Human EGF at 20 ng/ml was included as a positive control. ** Represents statistical significance with p-value <0.01. **** Represents statistical significance with p-value <0.0001.

These results can also be expressed as wound healing area, which is calculated by subtracting the wound area after 24 hours from total area before treatment.

Table 17 shows the raw data for wound area quantification in pixels.

TABLE 17

| | | | | % area | % relative | % wound |
|---|---|---|---|---|---|---|
| | | Basal | | wound | Area to | healing |
| | Test sample | (0 h) | 24 h | 24 h | Control 24 h | 24 h |
| Control | C-1 | 1282375 | 524321 | 40.887 | 87.034 | 12.966 |
| | C-2 | 1575418 | 531237 | 33.720 | 71.779 | 28.221 |
| | C-3 | 1555214 | 668398 | 42.978 | 91.485 | 8.515 |
| | C-4 | 1826622 | 1284618 | 70.328 | 149.703 | −49.703 |
| P2066 | EGF | 1324098 | 263185 | 19.877 | 42.310 | 57.690 |
| | 20 ng/ml −1 | | | | | |
| | EGF | 1346138 | 397433 | 29.524 | 62.846 | 37.154 |
| | 20 ng/ml −1 | | | | | |
| | EGF | 1134400 | 212198 | 18.706 | 39.818 | 60.182 |
| | 20 ng/ml −1 | | | | | |
| | EGF | 1741659 | 111545 | 6.405 | 13.633 | 86.367 |
| | 20 ng/ml −1 | | | | | |
| P2057 | RS-0198A, | 1472954 | 373510 | 26.358 | 53.978 | 46.022 |
| | 0.00001% −1 | | | | | |
| | RS-0198A, | 923265 | 130230 | 14.105 | 30.025 | 69.975 |
| | 0.00001% −2 | | | | | |
| | RS-0198A, | 1183450 | 137101 | 11.585 | 24.660 | 75.340 |
| | 0.00001% −3 | | | | | |
| | RS-0198A, | 1204073 | 214107 | 17.782 | 37.851 | 62.149 |
| | 0.0001% −4 | | | | | |
| P2057 | RS-0198A, | 1545419 | 1091524 | 70.630 | 150.346 | −50.346 |
| | 0.0001% −1 | | | | | |
| | RS-0198aA | 1444528 | 238054 | 16.480 | 35.080 | 64.920 |
| | 0.0001% −2 | | | | | |
| | RS-0198A, | 788334 | 138741 | 17.599 | 37.463 | 62.537 |
| | 0.0001% −3 | | | | | |
| | RS-0198A, | 1660505 | 337232 | 20.309 | 43.231 | 56.769 |
| | 0.0001% −4 | | | | | |
| P2057 | RS-0198A, | 1446515 | 340770 | 23.558 | 50.147 | 49.853 |
| | 0.001% −1 | | | | | |
| | RS-0198A, | 1511796 | 263702 | 17.443 | 37.130 | 62.870 |
| | 0.001% −2 | | | | | |
| | RS-0198A, | 1681864 | 277497 | 16.499 | 35.121 | 64.879 |
| | 0.001% −3 | | | | | |
| | RS-0198A, | 1952069 | 274000 | 14.036 | 29.879 | 70.121 |
| | 0.001% −4 | | | | | |
| P2058 | RS-0198B, | 1822242 | 260213 | 14.280 | 30.397 | 69.603 |
| | 0.0001% −1 | | | | | |
| | RS-0198B, | 1276067 | 675888 | 52.966 | 112.747 | −12.747 |
| | 0.0001% −2 | | | | | |
| | RS-0198B, | 1270058 | 500700 | 39.423 | 83.919 | 16.081 |
| | 0.0001% −3 | | | | | |
| | RS-0198B, | 1304906 | 384057 | 29.432 | 62.650 | 37.350 |
| | 0.0001% −4 | | | | | |
| P2058 | RS-0198B, | 1793164 | 229743 | 12.812 | 27.273 | 72.727 |
| | 0.001% −1 | | | | | |
| | RS-0198B, | 1402324 | 457572 | 32.630 | 69.457 | 30.543 |
| | 0.001% −2 | | | | | |
| | RS-0198B, | 1817912 | 511394 | 28.131 | 59.881 | 40.119 |
| | 0.001% −3 | | | | | |
| | RS-0198B, | 1424046 | 190205 | 13.357 | 28.432 | 71.568 |
| | 0.001% −4 | | | | | |
| P2058 | RS-0198B, | 1564837 | 470396 | 30.060 | 63.988 | 36.012 |
| | 0.01% −1 | | | | | |
| | RS-0198B, | 1178981 | 513294 | 43.537 | 92.675 | 7.325 |
| | 0.01% −2 | | | | | |
| | RS-0198B, | 1047236 | 149903 | 14.314 | 30.471 | 69.530 |
| | 0.01% −3 | | | | | |
| | RS-0198B, | 1366846 | 221529 | 16.207 | 34.500 | 65.500 |
| | 0.01% −4 | | | | | |
| | C-1 | 1186618 | 4099137 | 34.479 | 113.241 | −13.241 |
| | C-2 | 1460346 | 501789 | 34.361 | 112.852 | −12.852 |
| | C-3 | 1038651 | 266523 | 25.660 | 84.277 | 15.723 |
| | C-4 | 1500477 | 409488 | 27.291 | 89.630 | 13.370 |
| | C + EGF | 1188450 | 24456 | 27.291 | 89.630 | 10.370 |
| | 20 ng/ml 1 | | | | | |
| | C + EGF | 1278864 | 0 | 2.058 | 6.758 | 93.242 |
| | 20 ng/ml 2 | | | | | |
| | C + EGF | 1237384 | 0 | 0.000 | 0.000 | 100.000 |
| | 20 ng/ml 3 | | | | | |
| | C + EGF | 1227903 | 64389 | 5.244 | 17.222 | 82.778 |
| | 20 ng/ml 4 | | | | | |
| | RS 01221 | 1339416 | 318894 | 23.808 | 78.194 | 21.506 |
| | 0.001% −1 | | | | | |
| | RS 01221 | 1218663 | 331848 | 27.230 | 89.433 | 10.567 |
| | 0.001% −2 | | | | | |
| | RS 01221 | 973989 | 257094 | 26.396 | 86.693 | 13.307 |
| | 0.001% −3 | | | | | |
| | RS 01221 | 1205307 | 244566 | 20.291 | 66.641 | 33.359 |
| | 0.001% −4 | | | | | |
| | RS 01221 | 1225599 | 251426 | 20.515 | 67.376 | 32.624 |
| | 0.01% −1 | | | | | |
| | RS 01221 | 1193082 | 235176 | 19.712 | 64.739 | 35.261 |
| | 0.01% −2 | | | | | |
| | RS 01221 | 1195659 | 210021 | 17.565 | 57.690 | 42.310 |
| | 0.01% −3 | | | | | |
| | RS 01221 | 1153815 | 11757 | 1.020 | 3.349 | 96.651 |
| | 0.01% −4 | | | | | |

As shown in table 18, results showed that treatment of cultures of human keratinocytes for 24 h with RS-0198A, RS-0198B and hEGF enhanced wound healing compared with untreated controls. EGF at 20 ng/ml also boosted wound healing.

TABLE 18

| Test Sample | Dilution tested | Decreased wound area |
|---|---|---|
| RS-0198A | 0.00001% | 63.4 ± 15.7% |
| | 0.001% | 61.4 ± 17% |
| | 0.001% | 61.9 ± 15.7% |
| RS-0198B | 0.001% | 53.7 ± 15.7 |
| EGF | 20 ng/ml | 60.4 ± 15.7% |

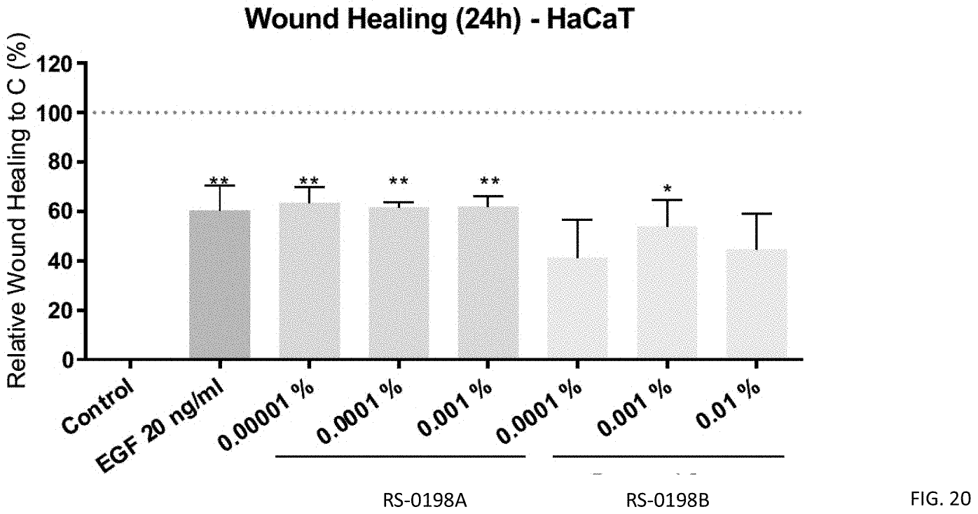

FIG. 20 is a bar graph representing wound healing relative to an untreated control after treatment for 24 hours of human keratinocytes (HaCaT) with RS-0198A at 0.00001%, 0.0001%, and 0.001% and RS-0198B at 0.0001%, 0.001%, and 0.01%. Human EGF at 20 ng/ml was included as a positive control. * Represents statistical significance with p-value <0.05. ** Represents statistical significance with p-value <0.01.

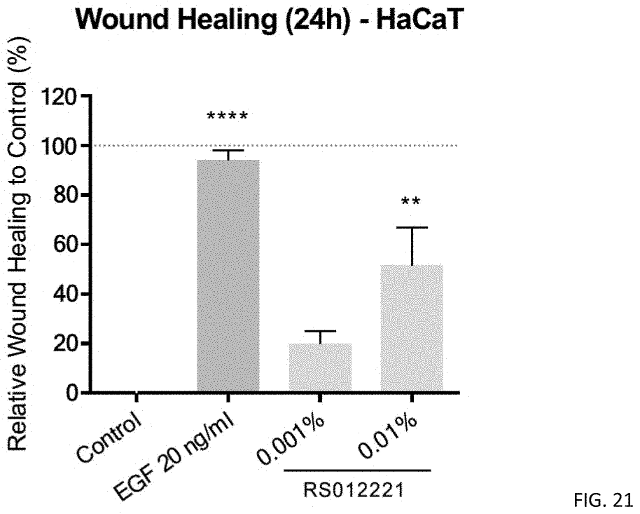

Results also showed that treatment of keratinocyte cultures for 24 hours with RS012221 at 0.01% significantly improved wound healing in human heratinocytes by 51.7±16.9% while exposure to EGF at 20 ng/ml induced significant wound healing by 94.0±8.6%. compared to the untreated control, FIG. 21 is a bar graph representing wound healing relative to an untreated control after treatment for 24 hours of human keratinocytes (HaCaT) with RS012221 at 0.001% and 0.01% concentrations. Human EGF at 20 ng/ml was included as a positive control. ** Represents statistical significance with p-value <0.01. **** Represents statistical significance with p-value <0.0001.

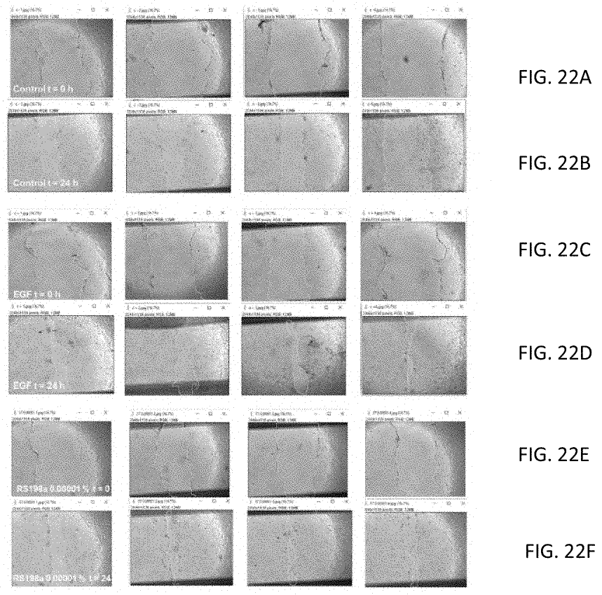

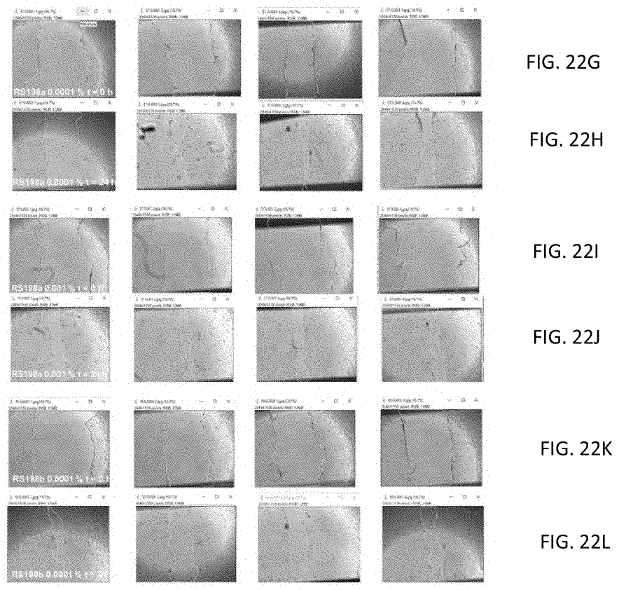

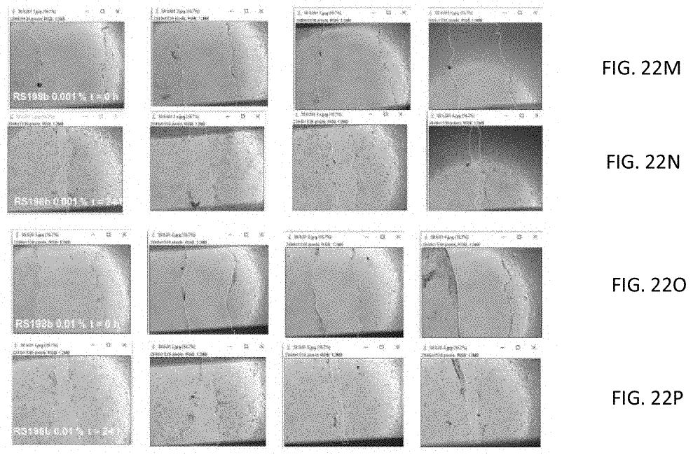

FIG. 22A-FIG. 22P show microscopic images of wound healing from scratches performed on human keratinocyte (HaCaT) monolayers immediately before (0 h) and 24 h after treatment. Human EGF at 20 ng/ml was included as a positive control. FIG. 22A Control, t=0 h; FIG. 22B Control, t=24 h; FIG. 22C, EGF t=0h; FIG. 22D, EGH t=24 h; FIG. 22E RS-0198A, 0.00001%, t=0 h; FIG. 22F, RS-0198A, 0.00001%, t=24 h; FIG. 22G, RS-0198A, 0.0001%, t=0 h; FIG. 22H RS-0198A, 0.0001%, t=24 h; FIG. 22I, RS-0198A, 0.001%, t=0 h; FIG. 22J, RS-0198A, 0.001%, t=24 h; FIG. 22K, RS-0198B, 0.0001%, t=0 h; FIG. 22L, RS-0198B, 0.0001%, t=24 h; FIG. 22M, RS-0198B, 0.001%, t=0 h; FIG. 22N, RS-0198B, 0.001%, t=24 h; FIG. 22O, RS-0198B, 0.01%; t=0 h; FIG. 22P, RS-0198B, 0.01%, T=24 h.

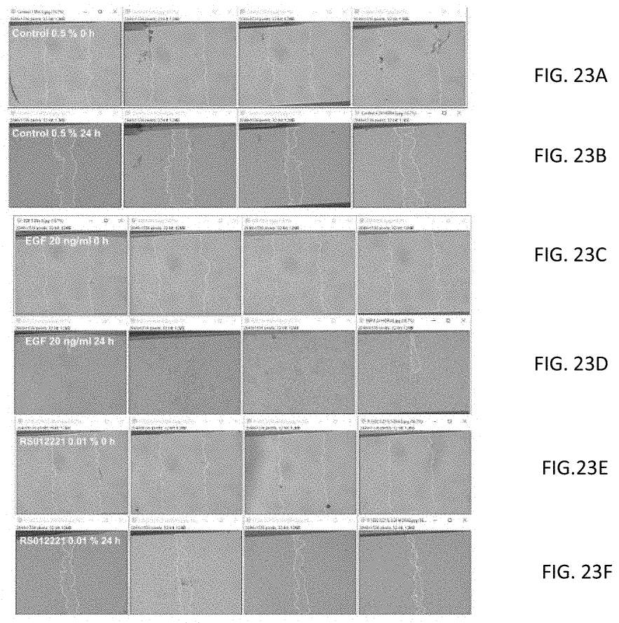

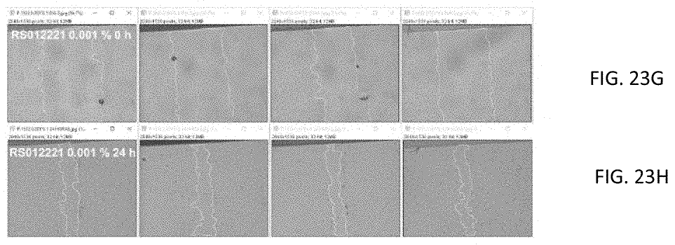

FIG. 23A-FIG. 23H show microscopic images of wound healing from scratches performed on human keratinocyts (HaCaT) monolayers immediately before (0 h) and 24 h after treatment. Human EGF at 20 ng/ml was included as a positive control. FIG. 23A, Control, 0.5%, t=0 h; FIG. 23B, Control, 0.5%, t=24 h; FIG. 23C EGF, 20 ng/ml, t=0 h; FIG. 23D, EGF, 20 ng/ml, t=24 h; FIG. 23E, RS012221, 0.01%, t=0 h; FIG. 23F, RS12221, 0.01%, t=24 h; FIG. 23G RS012221, 0.001, t=0 h; FIG. 23H, RS012221, 0.001%, t=24 h.

### Discussion

In this study the regenerating and stimulating effects of three test samples, namely RS-0198A, RS-0198B, RS12221 were compared to human EFG after a 24 hour treatment of human keratinocyte cultures in a wound healing (scratch) assay. Results showed that 24 hour treatment with RS-0198A at 0.00001%, 0.0001% and 0.001% significantly enhanced wound healing in human keratinocytes, compared to an untreated control. Results also showed that treatment with RS-0198B at 0.001% for the same time period induced a wound healing effect compared to an untreated control. RS012221 at 0.01% also significantly improved wound healing in human keratinocytes compared to the untreated control. The positive control (hEGF) likewise boosted wound healing compared to the untreated control.

In conclusion, the in vitro treatment of HaCaT human keratinocytes for 24 hr with RS-0198A, RS-0198B, and RS012221 in some concentrations tested displayed regenerating and stimulating capabilities through significant improvement of wound healing to a similar extent as did human EGF.

### Example 5: Effect of Formulation on Vaginal Microbiome

The vaginal channel, which is open to the environment through the external genital organs, is characterized by a dynamic ecosystem continuously changing throughout a woman's life. Changes in the ecosystem are generated by variations in the hormone levels, diseases, sexual activity, medications, and personal hygiene practices, among others. [Baki, G. & Alexander K S, Introduction to Cosmetic Formulation and Technology, Ch. 7, John Wiley & Sons, Hoboken, N.J., at 656, citing Eschenbach, D. et al. Clin. Infect. Dis. (2000) 30: 901-7]. During the reproductive years, the fluctuating hormone levels, which regulate the menstrual cycle, are influencing factors of the vaginal microbiota. [Id., citing Farage, M A & Maibach, H. Arch. Gynecol. Obstet. (2006) 273: 195-202].

The vaginal vault is colonized within 24 hours after birth, and it remains colonized until death. [Id.]. The vagina of healthy fertile women harbors an extensive number of bacteria, including Lactobacillus, Staphylococcus, Ureaplasma, Corynebacterium, Stretptococcus, Peptostreptococcus, Garnerella, Bacteroides, Mycoplasma, Enterococcus, Escherichia, Veillonella, Bifidobacterium species, and Candida [Id., citing Larsen, B. & Monif, G R. Cin. Infect. Dis. (2001) 32: e69-e77; Redondo-Lopez, V. et al. Rev. Infect. Dis. (1990) 12: 856-72] of which Lactobacillus spp dominate. [Id., citing Witkin, S S et al. Best Pract. Res. Cllin. Obstet. Gynaecol. (2007) 21 (3): 347-54].

In a fertile woman, the desquamated vaginal epithelial cells are stimulated by E2 signaling to release glycogen, a substrate that is metabolized by the native microflora. These bacteria degrade glycogen and convert it to lactic acid to create an acidic environment, which restricts the growth of pathogenic microorganisms. [Id., citing Boskey, E R, et al. Hum. Reprod. (2001) 16 (9): 1809-13]. This cycle is extremely important in order to maintain vaginal health. In healthy fertile women, the normal pH in the vagina ranges from 3.5 to 4.5, with a typical value of 4.2. [Id., citing Owen, D H & Katz, D F. Contraception (1999) 59 (2): 91-95]. The presence of bacteria along with glycogen and the acidic pH is a prerequisite for a healthy vaginal condition.

In the female reproductive tract, the innate immune system is modulated by two sex steroid hormones, estrogen and progesterone. A cyclical wave of neutrophils in the vaginal lumen is triggered by chemokines and correlates with circulating estrogen levels. Classical estrogen signaling in the female reproductive tract is activated through estrogen receptor a encoded by the EsrI gene. Using a mouse model in which Esr1 was conditionally ablated from the epithelial cells (Wnt7^{cre/+}) it was shown that in response to a physical stress, the lack of ESR1 caused the vaginal epithelium to deteriorate due to the absence of a protective cornified layer and a reduction in keratin production; an excess of neutrophil infiltration was also seen. Further, the histological presence of neutrophils was found to correlate with persistent enzymatic activity in the cervical-vaginal fluid. It was concluded that ESR1 activity in the vaginal epithelial cells is required to maintain proper structural integrity of the vagina and immune response. [Li, S. et al. Sci. Rep. (2018) 8(1): 11247]

In postmenopausal women, a reduction in E2 levels results in the production of fewer epithelial cells and a reduced glycogen content. With vaginal epithelial cell atrophy and a lack of lubrication, the vaginal tissue becomes vulnerable to physical damage, e.g. intercourse, and is subsequently susceptible to infection. Additionally, the composition of the vaginal microbiota often changes in postmenopausal women due to the reduction in the nutritional substrate for the microbiota. As a consequence the vaginal pH rises. [Baki, G. & Alexander K S, Introduction to Cosmetic Formulation and Technology, Ch. 7, John Wiley & Sons, Hoboken, N.J., at 656, citing Gupta, S. et al. Indian J. Pathol. Microbiol. (2006) 49: 457-61]. High pH promotes the growth of pathogenic bacteria, which may lead to infections and inflammation.

Free glycogen in genital fluid is associated with a genital microbiota dominated by Lactobacillus, suggesting glycogen is important for maintaining genital health. [Mirmonsef, P. et al. PLoS One (2014) 9 (7): e102467]. Without being limited by theory, treatments aimed at increasing genital free glycogen might impact Lactobacillus colonization.

### Example 6. Study to Evaluate the Anti-Inflammatory Capacity of the Composition of the Present Disclosure (Hereinafter “Product”) Measured by Interleukin 1-Alpha (IL-1A), Interleukin 1Beta (IL1B), Interleukin 6 (IL-6), Interleukin 8 (IL-8), and Tumor Necrosis Factor (TNF) Gene Expression Levels after Challenging Human HaCaT Keratinocytes with Pseudomonas aeruginosa Lipopolysaccharide (LPS) to Mimic an Inflammatory Response

Method Summary: HaCaT heratinocytes is cultured during 24 hours in the presence of Product at sub-cytotoxic doses. The inflammatory response is induced using E. coli lipopolysaccharide (LPS) at 50 μg/ml during the same period (24 h). After treatment, IL-1A, IL-1B, IL-6. IL-8 and TNF levels are quantified through qPCR with beta actin (ACTB) as the housekeeping gene.

Cell line: HaCaT, a long-lived, spontaneously transformed aneuploidy immortal human keratinocytes which is able to differentiate in vitro, derived from adult human skin, cell passage, 18-30. HaCaT cells have been shown to be suitable model to follow the release of inflammatory and repair mediators [Colombo, I., et al. Mediators Inflamm. (2017) 2017: 7435621].

Procedure. Confluent HaCaT cells cultured on a 75 cm2 Nunc™ EasYFlask™ (ThermoFisher Scientific) are detached by incubation with Trypsin-EDTA 0.5% without phenol red (Gibco) for 7 min at 37 C, 5% CO2. Trypsin is inactivated by adding 5 volumes of DMEM 1 g/L glucose (Gibco) supplemented with 10% FBS (Gibco) hereinafter D10 medium) and mixing thoroughly. The obtained cell suspension is mixed 1:1 with trypan blue 0.4% and incubated for 30 s at room temperature to allow the penetration of the dye inside the non-viable cells. Cell counting of the trypan blue-diluted cell suspension is performed in a Countess II Automated Cell Counter (Thermo Fisher Scientific)

Cells are diluted in D10 medium to a final density of 1.5E5 cells/ml. Cells are then seeded in a 6-well Clear Flat Bottom TC-treated Culture Microplate (Falcon) by adding 2 mL/well of the aforementioned cell suspension (3E5 cells/well). Cells are allowed to attach to the cell culture plastic by overnight incubation at 37 C, 5% CO2.

After the attachment period, D10 medium is removed and replaced by fresh D10 medium. Experimental conditions are as follows;
Control cells: are kept in D10 medium with no additional components.
Control+LPS: this group is exposed to LPS at 50 kg/ml in D10 medium.
   Treatment with the test sample: these conditions involved exposure to LPS at 50 μg/ml in D10 medium together with the tested sample.

For all experimental conditions, 2 mL of culture medium supplemented as indicated above is added per well. Cells are incubated in the presence of treatments for 24 h prior to RNA isolation.

After the incubation period, cell culture medium is completely removed from the wells and cells are rinsed with 2 mL Dulbecco's phosphate-buffered saline (DPBS) per well. Tissue culture plates are subjected to a freeze-thaw cycle at −80 C to allow the formation of crystals that help disrupt cells, and RNA is isolated from cell extracts with RNeasy Mini Kit as per the manufacturer's instructions, which are incorporated by reference herein. In brief, a specialized high-salt buffer system allows up to 100 μg of RNA longer than 200 bases to bind to the RNeasy silica membrane. Biological samples are first lysed and homogenized in the presence of a highly denaturing guanidine-thiocyanate-containing buffer, which immediately inactivates RNAses to ensure purification of intact RNA. Ethanol is added to provide appropriate binding conditions, and the sample is then applied to an RNeasy Mini spin column, where the total RNA binds to the membrane and contaminants are efficiently washed away. High-quality RNA is then eluted in 30-100 μl of water. The procedure provides an enrichment for mRNA since most RNAs <200 nucleotides (such as 5.8S rRNA, 5S rRNA and tRNAs, which together comprise 15-20% of total RNA) are selectively excluded. RNA is eluted from columns in a final volume of 30 μL of nuclease-free from the RNeasy Mini Kit.

Total RNA concentration from samples is quantified through absorbance at 260 nm in a Nanodrop 2000 spectrophotometer. RNA purity is assessed with 260/280 nm measurements.

Next, RNA is reverse transcribed into complementary DNA (cDNA) with PrimeScript™ RT reagent Kit as per manufacturer's instructions (https://www.takarabio.com/documents/User %20Manual/RR037A_e.v2008 Da.pdf), which are incorporated by reference herein, with a template input of 1000 ng in a 10±L reaction volume (final concentration: 100 ng/μL).

Finally, relative gene expression is assessed by quantitative polymerase chain reaction (qPCR) using the Premix ExTaq™ enzyme and mastermix according to supplier's guidelines, which are incorporated by reference herein. The included Tli RNase H, a heat resistant RNAse enzyme, minimizes PCR inhibition by removing residual mRNA in input cDNA. Briefly, 20 ng of input cDNA are loaded in a final reaction volume of 10 μL, with primers/probe concentrations of 500 nM/250 nM.

Gene Expression (qPCR)

For each technical replicate, the IL-A, IL-B, IL-6, IL-8 or TNF relative gene expression for the different experimental conditions versus the Control+LPS is calculated by the 2-Delta Delta C(T) Method (Livak, K J and Schmittgen, T D. Methods (2001) 25 (4): 402-8), with ACTB as housekeeping gene, and expressed as a percentage (%) versus Control+LPS.

Data are represented in two different ways of normalization. First, results are normalized to the untreated Control, in order to detect the capacity of LPS to induce/stimulate the expression of the genes of interest. Second, data re normalized versus the untreated Control+LPS, to show the capacity of the samples to attenuate the LPS-induced stimulation of gene expression. Data are represented in bar graphs as Mean±Standard Error of the Mean (SEM).

GraphPad Prism V9 software is used for the statistical analysis. Normalized data are analyzed using one-way ANOVA test followed by Dunnet's post hoc multiple comparison tests (relative gene expression vs. Control+LPS) or unpaired Student's t-test with Welch's correction (relative gene expression of Control+LPS vs untreated Control). Statistical significance is set at p<0.05.

One-way ANOVA, also called one-factor ANOVA, determines how a response is affected by one factor. In this experiment, the response to LPS and one test product is measured at 2 different concentrations. The conditions are the factor, and it is said to have three different levels, one for each experimental condition.

If there are only two levels of one factor (only two test condition) then a t-test should be used, like in the comparison between the Control and the Control+LPS, although the underlying math is the same for a t-test and one-way ANOVA with two groups.

ROUT (Robust regression and Outlier removal) method is used to identify outliers in the raw data, with a coefficient Q of 1%. The value of Q determines how aggressively the method defines the outlier and unless specified elsewhere, it is recommended to stick to 1% in this type of research experiment [Motulsky, H J and Brown, R E. BMC Bioinformatics. (2006) 7: 123].

Asterisks (*) over column bars in the graphs of results indicate statistical significance between the corresponding comparisons, and is indicated as * for p<0.05; ** for p<0.01, *** for p<0.001, and *** for p<0.0001.

### Example 7. Study to Determine Whether the Test Product Stimulates a Proangiogenic Response Mediated by VEGFA in Human Umbilical Vein Endothelial Cells (HUVEC), Derived from Neonatal Skin

HUVECs have been considered a general model for endothelial cells both in normal and diseased conditions. HUVECs express endothelial markers, e.g., ICAM-1, VCAM, and selectins, as well as signaling molecules associated with vascular physiology, such as NO (Boerma, M. et al Blood Coagul. Fibrinolysis (2006) 17 (3): 173-80; Caniuguir, A. et al. Placenta (2016) 41: 14-26). HUVECs have been shown to be responsive to physiological and/or pathological stimuli, such as high glucose, LPS and shear stress [Patel, H. et al. Cardiovascular Diabetology (2013) 12: article 142, Walshe, T E et al Arterioscler. Thromb. Vasc. Biol. (2013) 33 (11): 2608-17; 2013; Jang, J. et al. Sci Rep. (2017) 7: 41612].

Experimental design: application of the test product during 24 h followed by subsequent measurement of VEGFA expression by qPCR.

Procedure: Confluent HUVEC cultured on a 75 cm2 Nunc™ EasYFlask™ (Thermo Fisher Scientific) are detached by incubation with Trypsin-EDTA 0.5% without phenol red (Gibco) for 5 min at 37 C, 5% CO2. Trypsin is then inactivated by adding five volumes of trypsin neutralization solution and mixing thoroughly. The obtained cell suspension is mixed 1:1 with trypan blue 0.4% and incubated for 20 s at room temperature to allow penetration of the dye inside the non-viable cells. Cell counting of the trypan blue-diluted cell suspension is performed in a Countess II automated cell counter (Thermo Fisher Scientific).

Cells are diluted in D10 medium to a final density of 1.5E5 cells/ml. Cells are then seeded in a 6 well Clear Flat Bottom TC-treated Culture Microplate (Falcon) by adding 2 mL/well of the aforementioned cell suspension (3E5 cells/well). Cells are allowed to attach to the cell culture plastic by incubation overnight at 37 C, 5% CO2.

After the attachment period, D10 medium is removed and replaced by fresh D10 medium. Experimental conditions:

Control cells belonging to this group are kept in D10 medium with no additional components.

Treatment with the tested sample: these experimental conditions involve the tested sample at subcytotoxic doses.

For all experimental conditions, 2 mL of culture medium supplemented as indicated above are added per well. Cells are incubated in the presence of treatments for 24 h prior to RNA isolation.

After the incubation period, cell culture medium is completely removed from the wells and cells are rinsed well with 2 ml DPBS. Then DPBS is also discarded and 350 μL RLT buffer from an RNeasy Mini Kit are added per well. Buffer RLT, which is a proprietary component of RNeasy Kits, contains a high concentration of guanidine isothiocycanate, which supports the binding of RNA to the silica membrane. Tissue culture plates are subjected to a freeze thaw cycle at −80 C to allow the formation of crystals to help disrupt cells and RNA is isolated from cell extracts with RNeasy Mini Kit as per manufacturer's instructions, which are incorporated by reference herein. In brief, a specialized high-salt buffer system allows up to 100 μg of RNA longer than 200 bases to bind to the RNeasy silica membrane. Biological samples are first lysed and homogenized in the presence of a highly denaturing guanidine-thiocyanate-containing buffer, which immediately inactivates RNAses to ensure purification of intact RNA. Ethanol is added to provide appropriate binding conditions, and the sample is then applied to an RNeasy Mini spin column, where the total RNA binds to the membrane and contaminants are efficiently washed away. High-quality RNA is then eluted in 30-100 μl of water. The procedure provides an enrichment for mRNA since most RNAs <200 nucleotides (such as 5.8S rRNA, 5S rRNA and tRNAs, which together comprise 15-20% of total RNA) are selectively excluded. RNA is eluted from columns in a final volume of 30 μL of nuclease-free from the RNeasy Mini Kit.

Total RNA concentration from samples is quantified through absorbance at 260 nm in a Nanodrop 2000 spectrophotometer. RNA purity is assessed with 260/280 nm measurements.

Next, RNA is reverse transcribed into complementary DNA (cDNA) with PrimeScript™ RT reagent Kit as per manufacturer's instructions, which are incorporated by reference herein (https://www.takarabio.com/documents/User %20Manual/RR037A_e.v2008Da.pdf), with a template input of 1000 ng in a 10±L reaction volume (final concentration: 100 ng/μL)

Finally, relative gene expression is assessed by quantitative polymerase chain reaction (qPCR) using the Premix ExTaq™ enzyme and mastermix according to supplier's guidelines, which are incorporated by reference herein. The included Tli RNase H, a heat resistant RNAse enzyme, minimizes PCR inhibition by removing residual mRNA in input cDNA. Briefly, 20 ng of input cDNA are loaded in a final reaction volume of 10 μL, with primers/probe concentrations of 500 nM/250 nM.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A cosmetic composition formulated for topical application comprising
a. a water-based gel component comprising hyaluronic acid;
b. a botanical ingredient component comprising decarboxylated cannabidiol (CBD) representing about 20% of an isolate, and
c. a cosmetic composition stabilizing system comprising arginine, p-anisic acid, and levulinic acid,
wherein
the composition is a slightly viscous non-occluded water based liquid;
pH of the finished product ranges from about 4.0 to about 5.0 inclusive; and
the composition is not psychoactive.

2. The cosmetic composition according to claim 1, wherein the hyaluronic acid comprises about 0.10 to about 0.50 wt %, inclusive, low molecular weight hyaluronic acid (LMWHA) and about 0.50 to about 1.50 wt %, inclusive, high molecular weight hyaluronic acid (HMWHA), wherein ratio of the HMWHA to MMWHA ranges from 1:0.07-to 1:1, inclusive.

3. The cosmetic composition according to claim 1, wherein molecular weight of the HMWHA ranges from 1,000-1,800 kDa, inclusive.

4. The cosmetic composition according to claim 3, wherein the molecular weight of the HMWHA is at least 1000, 1100, 1200, 1300, 1400, 1500, 1600, 1700, or 1800 kDa.

5. The cosmetic composition according to claim 1, wherein molecular weight of the LMWHA is at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, or at least 9 kDa to less than 10 kDa.

6. The cosmetic composition according to claim 1, wherein the decarboxylated CBD representing 20% of an isolate is in form of a THC free nano-infused water soluble powder.

7. The cosmetic composition according to claim 1, wherein the cosmetic stabilizing system comprises about 0.25 wt % to about 1.00 wt %, inclusive, arginine levulinate and about 0.05 wt % to about 0.50 wt %, inclusive arginine anisate.

8. The cosmetic composition according to claim 1, wherein viscosity of the composition ranges from 5000 to 7500 centipoise, inclusive at room temperature.

9. The cosmetic composition according to claim 1, wherein the composition comprises about 1.0 wt % to about 5.0 wt %, inclusive of the THC-free nanoinfused water-soluble powder comprising about 20% decarboxylated CBD.

10. A method for promoting and maintaining vaginovulval tissue vitality and tissue health in a female subject in need thereof comprising administering topically to the subject a cosmetic composition comprising
a. a water-based gel component comprising hyaluronic acid;
b. a botanical ingredient component comprising a decarboxylated cannabidiol (CBD) representing 20% w/w of an isolate; and
c. a cosmetic composition stabilizing system comprising arginine, p-anisic acid, and levulinic acid,
wherein
the composition is a slightly viscous non-occluded water based liquid;
pH of the composition ranges from about 4.0 to about 5.0, inclusive;
the composition is not psychoactive;
therapeutic effects of the water-based gel component and the botanical ingredient component may be complementary; and
the composition promotes and maintains vaginovulval tissue vitality and tissue health.

11. The method according to claim 10, wherein:
a. the female subject in need thereof is a female subject susceptible to or experiencing vaginovulval symptoms of trauma, insult or injury or
b. a female subject experiencing genitourinary symptoms of trauma, insult or injury.

12. The method according to claim 10, wherein
a. the female subject in need thereof is a menopausal subject; or
b. the female subject in need thereof is a diabetic subject; or
c. the female subject in need thereof is a subject that will undergo, is undergoing or has undergone treatment comprising radiation therapy to treat a gynecologic cancer; or
d. the female subject in need thereof is a breast cancer survivor.

13. The method according to claim 12, wherein the gynecologic cancer is an endometrial cancer, a cervical cancer; an ovarian cancer; or a vulvar cancer.

14. The method according to claim 10, wherein parameters of vaginovulval tissue vitality include one or more of improved tissue strength, appropriate vaginal pH; reduced susceptibility to trauma/mechanical insult, reduced inflammation, reduced itching, improved wound healing, and improved tissue elasticity.

15. The method according to claim 10, wherein the cosmetic composition is effective to restore wounded tissue to a healthy tissue.

16. The method according to claim 15, wherein the composition, compared to an untreated control:
a. modulates vaginal pH; or
b. improves healing and rejuvenation of wounded tissue; or
c. reduces susceptibility to trauma or mechanical injury; or
d. reduces symptoms of trauma, insult or injury; or
e. reduces clinical signs of dryness and insufficient hydration (e.g., loss of elasticity, inflammation); or
f. reduces itching; or
g. a combination thereof.

17. The method according to claim 16, wherein improving healing and rejuvenation of wounded tissue comprises improving tissue strength.

18. The method according to claim 16, wherein symptoms of trauma, insult or injury comprise one or more of dryness, burning, irritation, discomfort or pain.

19. The method according to claim 16, wherein clinical signs of dryness and insufficient hydration include loss of elasticity, inflammation or both.

20. The method according to claim 10, wherein the hyaluronic acid comprises about 0.10 wt % to about 0.50 wt %, inclusive, low molecular weight hyaluronic acid (LMWHA) and about 0.50 wt % to about 1.50 wt %, inclusive, high molecular weight hyaluronic acid (HMWHA), wherein ratio of the HMWHA to LMWHA ranges from 1:0.7 to 1:1, inclusive.

21. The method according to claim 10, wherein molecular weight of the HMWHA ranges from 1,000-1,800 kDa, inclusive.

22. The method according to claim 21, wherein the molecular weight of the HMWHA is at least 1000, 1100, 1200, 1300, 1400, 1500, 1600, 1700, or 1800 kDa.

23. The method according to claim 20, wherein molecular weight of the LMWHA is at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, or at least 9 kDa to less than 10 kDa.

24. The method according to claim 10, wherein the decarboxylated CBD is in form of a THC free nano-infused water soluble powder.

25. The method according to claim 10, wherein the cosmetic stabilizing system comprises about 0.25 wt % to about 1.00 wt %, inclusive, arginine levulinate and about 0.05 wt % to about 0.50 wt %, inclusive arginine anisate.

26. The method according to claim 11, wherein viscosity of the composition ranges from 5000 to 7500 centipoise, inclusive at room temperature.

27. The method according to claim 11, wherein the composition comprises about 1.0% to about 5.0%, inclusive of the THC-free nanoinfused water-soluble powder comprising about 20% decarboxylated CBD.

28. A method for promoting and maintaining perianal tissue vitality and tissue health in a subject with a hemorrhoidal disease, the method comprising administering topically to the subject a cosmetic composition comprising
a. a water-based gel component comprising hyaluronic acid;
b. a botanical ingredient component comprising a decarboxylated cannabidiol (CBD) representing 20% w/w of an isolate; and
c. a cosmetic composition stabilizing system comprising arginine, p-anisic acid, and levulinic acid,
wherein
the composition is a slightly viscous non-occluded water based liquid;
pH of the composition ranges from about 4.0 to about 5.0, inclusive;
the composition is not psychoactive;
therapeutic effects of the water-based gel component and the botanical ingredient component may be complementary; and
the composition reduces one or more cutaneous symptoms of the hemorrhoidal disease.

29. The method according to claim 28, wherein the hemorrhoidal disease comprises external hemorrhoidal tissue.

30. The method according to claim 28, wherein the composition, compared to an untreated control:
a. improves healing and rejuvenation of the external hemorrhoidal tissue; or
b. reduces susceptibility of the external hemorrhoidal tissue to trauma or mechanical injury; or
c. reduces symptoms of trauma, insult or injury; or
d. reduces clinical signs of dryness and insufficient hydration of the external hemorrhoidal tissue; or
e. reduces itching; or
f. a combination thereof.

31. The method according to claim 30, wherein improving healing and rejuvenation of the external hemorrhoidal tissue comprises improving tissue strength.

32. The method according to claim 30, wherein symptoms of trauma, insult or injury comprise one or more of dryness, burning, irritation, discomfort or pain.

33. The method according to claim 30, wherein clinical signs of dryness and insufficient hydration include loss of elasticity, inflammation or both.

34. The method according to claim 28, wherein the hyaluronic acid comprises about 0.10 wt % to about 0.50 wt %, inclusive, low molecular weight hyaluronic acid (LMWHA) and about 0.50 wt % to about 1.50 wt %, inclusive, high molecular weight hyaluronic acid (HMWHA), wherein ratio of the HMWHA to LMWHA ranges from 1:0.7 to 1:1, inclusive.

35. The method according to claim 34, wherein molecular weight of the HMWHA ranges from 1,000-1,800 kDa, inclusive.

36. The method according to claim 35, wherein the molecular weight of the HMWHA is at least 1000, 1100, 1200, 1300, 1400, 1500, 1600, 1700, or 1800 kDa.

37. The method according to claim 34, wherein molecular weight of the LMWHA is at least 0.1 kDa to less than 10 kDa, at least 0.5 kDa to less than 10 kDa, at least 1 kDa to less than 10 kDa, at least 2 kDa to less than 10 kDa, at least 3 kDa to less than 10 kDa, at least 4 kDa to less than 10 kDa, at least 5 kDa to less than 10 kDa, at least 6 kDa to less than 10 kDa, at least 7 kDa to less than 10 kDa, at least 8 kDa to less than 10 kDa, or at least 9 kDa to less than 10 kDa.

38. The method according to claim 28, wherein the decarboxylated CBD is in form of a THC free nano-infused water soluble powder.

39. The method according to claim 28, wherein the cosmetic stabilizing system comprises about 0.25 wt % to about 1.00 wt %, inclusive, arginine levulinate and about 0.05 wt % to about 0.50 wt %, inclusive arginine anisate.

40. The method according to claim 28, wherein viscosity of the composition ranges from 5000 to 7500 centipoise, inclusive at room temperature.

41. The method according to claim 28, wherein the composition comprises about 1.0% to about 5.0%, inclusive of the THC-free nanoinfused water-soluble powder comprising about 20% decarboxylated CBD.
